SEQUENCE LISTING (118 sequences)

SEQ ID NO: 1 - protein, 573 aa, synthetic construct
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGSSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERGSHHHHHH

SEQ ID NO: 2 - protein, 597 aa, synthetic construct
MLLFVLTCLLAVFPAISTKSPIFGPEQVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNGSQVQLVESGGGLAQAGGSLRLSCAASGRTFSMDPMAWFRQPPGKEREFVAAGSSTGRTTYYADSVKGRFTISRDNAKNTVYLQMNSLKPEDTAVYYCAAAPYGANWYRDEYDYWGQGTQVTVSSSGKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGSSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERGSHHHHHH

SEQ ID NO: 3 - protein, 598 aa, synthetic construct
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNGSQVKLEESGGGLVQAGGSLRLSCAASGRTFSMYRMGWFRQAPGKEREFVGVITRNGSSTYYADSVKGRFTISRDNAKNTVYLQMNSLKPEDTALYYCAATSGSSYLDAAHVYDYWGQGTQVTVSSSGKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGSSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERGSHHHHHH

SEQ ID NO: 4 - protein, 597 aa, synthetic construct
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNGSQVKLEESGGGLVQAGGSLRLSCAASRLTFSTYHMGWFRQAPGKEREFVAALSWSGGTTYYADSVKGRFGISRDNAKNTVYLQMNSLKPEDTAVYYCASGGVLATMNSDEYDYWGQGTQVTVSSSGKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGSSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERGSHHHHHH

SEQ ID NO: 5 - protein, 587 aa, synthetic construct
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNGSEIVLTQSPGTLSLSPGERATLSCRASQSVSSSYLAWYQQKPGQAPRLLIYGASSRATGIPDRFSGSGSGTETTLTISRLEPEDFAVYYCQQYGSSTWTFGQGTKVEIKRTVAASGKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGSSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERGSHHHHHH

SEQ ID NO: 6 - protein, 587 aa, synthetic construct
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKGRTFSMDPKSLYKQIGLYPVLVIDGSSTGRTTGYVNPNYTGRIRLDIQGTGQLLFSVVINQLRLSDAGQYLCAAAPYGANWYRDEYDYKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGSSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERGSHHHHHH

SEQ ID NO: 7 - protein, 588 aa, synthetic construct
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKGRTFSMYRKSLYKQIGLYPVLVIDITRNGSSTGYVNPNYTGRIRLDIQGTGQLLFSVVINQLRLSDAGQYLCAATSGSSYLDAAHVYDYKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGSSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERGSHHHHHH

SEQ ID NO: 8 - protein, 542 aa, synthetic construct
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNGSIEEQAKTFLDKFNHEAEDLFYQSSLASWNYNTNITEENVQNMNNAGDKWSAFLKEQSTLAQMYPLQEISGKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGSSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERGSHHHHHH

SEQ ID NO: 9 - protein, 604 aa, synthetic construct
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNGSIEEQAKTFLDKFNHEAEDLFYQSSLASWNYNTNITEENVQNMNNAGDKWSAFLKEQSTLAQMYPLQEIQNLTVKLQLQALQQNGSGGGGGMTQGFWENSMLTDPGNVQKAVCHPTAWDLGKGDFRILMCTSGKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGSSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERGSHHHHHH

SEQ ID NO: 10 - protein, 593 aa, synthetic construct
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNGSEVQLVQSGAEVKKSGESLKISCKGSGYSFTSYWIGWVRQMPGKGLEWMGIFYPGDSSTRYSPSFQGQVTISADKSVNTAYLQWSSLKASDTAMYYCARRRNWGNAFDIWGQGTMVTVSSSGKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGSSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERGSHHHHHH

SEQ ID NO: 11 - protein, 596 aa, synthetic construct
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNGSQVQLVESGGGVVQPGRSLRLSCAASGFSFSNYGMHWVRQAPGKGLEWVALIWYDGSNEDYTDSVKGRFTISRDNSKNTLYLQMNSLRAEDTAVYYCARWGMVRGVIDVFDIWGQGTVVTVSSSGKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGSSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERGSHHHHHH

SEQ ID NO: 12 - protein, 587 aa, synthetic construct
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNGSDIQMTQSPSSVSASVGDRVTITCRASQGISSWLAWYQHKPGKAPKLLIYAASSLQSGVPSRFSGSGSGTDFTLTISSLQPEDFATYYCQQANSFPWTFGQGTKVEILGQPKSSSGKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGSSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERGSHHHHHH

SEQ ID NO: 13 - protein, 579 aa, synthetic construct
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNQAVVTQESALTTSPGETVTLTCRSSNGAVTSRNYANWVQEKPDHLFTGLIGGTNNRAPGVPARFSGSLIGDKAALSITGAQTEDEAIYFCALWYSNRWVFGGGTKLTVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGSSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERGSHHHHHH

SEQ ID NO: 14 - protein, 585 aa, synthetic construct
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNQVQLQQSDAELVKPGASVKISCKASGYTFTDHAIHWVKQKPEQGLEWIGYISPGNDDIKYNEKFKGKATLTADTSSSTAYMQLNSLTSEDSAVYFCKVLRRFAYWGQGTLVTVSAKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGSSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERGSHHHHHH

SEQ ID NO: 15 - protein, 595 aa, synthetic construct
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNQVKLEESGGGLVQAGGSLRLSCADSERTFRIYTMAWFRQAPGKERDFVAAISWSGGSTYYADSVKGRFTISRDNAKNTVYLPMNSLKPDDTAVYYCASGGVLSTGSQSDSEYDFWGQGTQVTVSSKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGSSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERGSHHHHHH

SEQ ID NO: 16 - protein, 581 aa, synthetic construct
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDIVLTQSPASLAVSLGQRATISCRASKSVSTSGYSYMHWYQQKPGQPPKLLIYLASNLESGVPARFSGSGSGTDFTLNIHPVEEEDAATYYCQHSRELPRTFGGGTKLEIKKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGSSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERGSHHHHHH

SEQ ID NO: 17 - protein, 588 aa, synthetic construct
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNEVQLQQSGPELVKPGASVKISCKASGYTFTDYNMWVKQSHGKSLEWIGYIYPYNGGTGYNQKFKSKATLTVDNSSSTAYMELRSLTSEDSAVYYCARNYYGSSWFAYWGQGTLVTVSAKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGSSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERGSHHHHHH

SEQ ID NO: 18 - protein, 595 aa, synthetic construct
MRLYLFTLLVTVFSGVSTKSPIFGPQEVSSIEGDSVSITCYYPDTSVNRHTRKYWCRQGASGMCTTLISSNGYLSKEYSGRANLINFPENNTFVINIEQLTQDDTGSYKCGLGTSNRGLSFDVSLEVSQVPELPSQVQLVESGGGLAQAGGSLRLSCAASGRTFSMDPMAWFRQPPGKEREFVAAGSSTGRTTYYADSVKGRFTISRDNAKNTVYLQMNSLKPEDTAVYYCAAAPYGANWYRDEYDYWGQGTQVTVSSAPEPELLYKDLRSSVTFECDLGREVANEAKYLCRMNKETCDVIINTLGKRDPDFEGRILITPKDDNGRFSVLITGLRKEDAGHYQCGAHSSGLPQEGWPIQTWQLFVNEESTIPNRRSVVKGVTGGSVAIACPYNPKESSSLKYWCRWEGDGNGHCPVLVGTQAQVQEEYEGRLALFDQPGNGTYTVILNQLTTEDAGFYWCLTNGDSRWRTTIELQVAEATREPNLEVTPQNATAVLGETFTVSCHYPCKFYSQEKYWCKWSNKGCHILPSHDEGARQSSVSCDQSSQLVSMTLNPVSKEDEGWYWCGVKQGQTYGETTAIYIAVEERGSHHHHHH

SEQ ID NO: 19 - protein, 596 aa, synthetic construct
MRLYLFTLLVTVFSGVSTKSPIFGPQEVSSIEGDSVSITCYYPDTSVNRHTRKYWCRQGASGMCTTLISSNGYLSKEYSGRANLINFPENNTFVINIEQLTQDDTGSYKCGLGTSNRGLSFDVSLEVSQVPELPSQVKLEESGGGLVQAGGSLRLSCAASGRTFSMYRMGWFRQAPGKEREFVGVITRNGSSTYYADSVKGRFTISRDNAKNTVYLQMNSLKPEDTALYYCAATSGSSYLDAAHVYDYWGQGTQVTVSSAPEPELLYKDLRSSVTFECDLGREVANEAKYLCRMNKETCDVIINTLGKRDPDFEGRILITPKDDNGRFSVLITGLRKEDAGHYQCGAHSSGLPQEGWPIQTWQLFVNEESTIPNRRSVVKGVTGGSVAIACPYNPKESSSLKYWCRWEGDGNGHCPVLVGTQAQVQEEYEGRLALFDQPGNGTYTVILNQLTTEDAGFYWCLTNGDSRWRTTIELQVAEATREPNLEVTPQNATAVLGETFTVSCHYPCKFYSQEKYWCKWSNKGCHILPSHDEGARQSSVSCDQSSQLVSMTLNPVSKEDEGWYWCGVKQGQTYGETTAIYIAVEERGSHHHHHH

SEQ ID NO: 20 - protein, 595 aa, synthetic construct
MRLYLFTLLVTVFSGVSTKSPIFGPQEVSSIEGDSVSITCYYPDTSVNRHTRKYWCRQGASGMCTTLISSNGYLSKEYSGRANLINFPENNTFVINIEQLTQDDTGSYKCGLGTSNRGLSFDVSLEVSQVPELPSQVKLEESGGGLVQAGGSLRLSCAASRLTFSTYHMGWFRQAPGKEREFVAALSWSGGTTYYADSVKGRFGISRDNAKNTVYLQMNSLKPEDTAVYYCASGGVLATMNSDEYDYWGQGTQVTVSSAPEPELLYKDLRSSVTFECDLGREVANEAKYLCRMNKETCDVIINTLGKRDPDFEGRILITPKDDNGRFSVLITGLRKEDAGHYQCGAHSSGLPQEGWPIQTWQLFVNEESTIPNRRSVVKGVTGGSVAIACPYNPKESSSLKYWCRWEGDGNGHCPVLVGTQAQVQEEYEGRLALFDQPGNGTYTVILNQLTTEDAGFYWCLTNGDSRWRTTIELQVAEATREPNLEVTPQNATAVLGETFTVSCHYPCKFYSQEKYWCKWSNKGCHILPSHDEGARQSSVSCDQSSQLVSMTLNPVSKEDEGWYWCGVKQGQTYGETTAIYIAVEERGSHHHHHH

SEQ ID NO: 21 - protein, 580 aa, synthetic construct
MRLYLFTLLVTVFSGVSTKSPIFGPQEVSSIEGDSVSITCYYPDTSVNRHTRKYWCRQGASGMCTTLISSNGYLSKEYSGRANLINFPENNTFVINIEQLTQDDTGSYKCGLGTSNRGLSFDVSLEVSQVPELPSEIVLTQSPGTLSLSPGERATLSCRASQSVSSSYLAWYQQKPGQAPRLLIYGASSRATGIPDRFSGSGSGTETTLTISRLEPEDFAVYYCQQYGSSTWTFGQGTKVEIKAPEPELLYKDLRSSVTFECDLGREVANEAKYLCRMNKETCDVIINTLGKRDPDFEGRILITPKDDNGRFSVLITGLRKEDAGHYQCGAHSSGLPQEGWPIQTWQLFVNEESTIPNRRSVVKGVTGGSVAIACPYNPKESSSLKYWCRWEGDGNGHCPVLVGTQAQVQEEYEGRLALFDQPGNGTYTVILNQLTTEDAGFYWCLTNGDSRWRTTIELQVAEATREPNLEVTPQNATAVLGETFTVSCHYPCKFYSQEKYWCKWSNKGCHILPSHDEGARQSSVSCDQSSQLVSMTLNPVSKEDEGWYWCGVKQGQTYGETTAIYIAVEERGSHHHHHH

SEQ ID NO: 22 - protein, 591 aa, synthetic construct
MRLYLFTLLVTVFSGVSTKSPIFGPQEVSSIEGDSVSITCYYPDTSVNRHTRKYWCRQGASGMCTTLISSNGYLSKEYSGRANLINFPENNTFVINIEQLTQDDTGSYKCGLGTSNRGLSFDVSLEVSQVPELPSEVQLVQSGAEVKKSGESLKISCKGSGYSFTSYWIGWVRQMPGKGLEWMGIFYPGDSSTRYSPSFQGQVTISADKSVNTAYLQWSSLKASDTAMYYCARRRNWGNAFDIWGQGTMVTVSSAPEPELLYKDLRSSVTFECDLGREVANEAKYLCRMNKETCDVIINTLGKRDPDFEGRILITPKDDNGRFSVLITGLRKEDAGHYQCGAHSSGLPQEGWPIQTWQLFVNEESTIPNRRSVVKGVTGGSVAIACPYNPKESSSLKYWCRWEGDGNGHCPVLVGTQAQVQEEYEGRLALFDQPGNGTYTVILNQLTTEDAGFYWCLTNGDSRWRTTIELQVAEATREPNLEVTPQNATAVLGETFTVSCHYPCKFYSQEKYWCKWSNKGCHILPSHDEGARQSSVSCDQSSQLVSMTLNPVSKEDEGWYWCGVKQGQTYGETTAIYIAVEERGSHHHHHH

SEQ ID NO: 23 - protein, 594 aa, synthetic construct
MRLYLFTLLVTVFSGVSTKSPIFGPQEVSSIEGDSVSITCYYPDTSVNRHTRKYWCRQGASGMCTTLISSNGYLSKEYSGRANLINFPENNTFVINIEQLTQDDTGSYKCGLGTSNRGLSFDVSLEVSQVPELPSQVQLVESGGGVVQPGRSLRLSCAASGFSFSNYGMHWVRQAPGKGLEWVALIWYDGSNEDYTDSVKGRFTISRDNSKNTLYLQMNSLRAEDTAVYYCARWGMVRGVIDVFDIWGQGTVVTVSSAPEPELLYKDLRSSVTFECDLGREVANEAKYLCRMNKETCDVIINTLGKRDPDFEGRILITPKDDNGRFSVLITGLRKEDAGHYQCGAHSSGLPQEGWPIQTWQLFVNEESTIPNRRSVVKGVTGGSVAIACPYNPKESSSLKYWCRWEGDGNGHCPVLVGTQAQVQEEYEGRLALFDQPGNGTYTVILNQLTTEDAGFYWCLTNGDSRWRTTIELQVAEATREPNLEVTPQNATAVLGETFTVSCHYPCKFYSQEKYWCKWSNKGCHILPSHDEGARQSSVSCDQSSQLVSMTLNPVSKEDEGWYWCGVKQGQTYGETTAIYIAVEERGSHHHHHH

SEQ ID NO: 24 - protein, 579 aa, synthetic construct
MRLYLFTLLVTVFSGVSTKSPIFGPQEVSSIEGDSVSITCYYPDTSVNRHTRKYWCRQGASGMCTTLISSNGYLSKEYSGRANLINFPENNTFVINIEQLTQDDTGSYKCGLGTSNRGLSFDVSLEVSQVPELPSDIQMTQSPSSVSASVGDRVTITCRASQGISSWLAWYQHKPGKAPKLLIYAASSLQSGVPSRFSGSGSGTDFTLTISSLQPEDFATYYCQQANSFPWTFGQGTKVEIKAPEPELLYKDLRSSVTFECDLGREVANEAKYLCRMNKETCDVIINTLGKRDPDFEGRILITPKDDNGRFSVLITGLRKEDAGHYQCGAHSSGLPQEGWPIQTWQLFVNEESTIPNRRSVVKGVTGGSVAIACPYNPKESSSLKYWCRWEGDGNGHCPVLVGTQAQVQEEYEGRLALFDQPGNGTYTVILNQLTTEDAGFYWCLTNGDSRWRTTIELQVAEATREPNLEVTPQNATAVLGETFTVSCHYPCKFYSQEKYWCKWSNKGCHILPSHDEGARQSSVSCDQSSQLVSMTLNPVSKEDEGWYWCGVKQGQTYGETTAIYIAVEERGSHHHHHH

SEQ ID NO: 25 - protein, 581 aa, synthetic construct
MRLYLFTLLVTVFSGVSTKSPIFGPQEVSSIEGDSVSITCYYPDTSVNRHTRKYWCRQGASGMCTTLISSNGYLSKEYSGRANLINFPENNTFVINIEQLTQDDTGSYKCGLGTSNRGLSFDVSLEVSQVPELPSQAVVTQESALTTSPGETVTLTCRSSNGAVTSRNYANWVQEKPDHLFTGLIGGTNNRAPGVPARFSGSLIGDKAALSITGAQTEDEAIYFCALWYSNRWVFGGGTKLTVLAPEPELLYKDLRSSVTFECDLGREVANEAKYLCRMNKETCDVIINTLGKRDPDFEGRILITPKDDNGRFSVLITGLRKEDAGHYQCGAHSSGLPQEGWPIQTWQLFVNEESTIPNRRSVVKGVTGGSVAIACPYNPKESSSLKYWCRWEGDGNGHCPVLVGTQAQVQEEYEGRLALFDQPGNGTYTVILNQLTTEDAGFYWCLTNGDSRWRTTIELQVAEATREPNLEVTPQNATAVLGETFTVSCHYPCKFYSQEKYWCKWSNKGCHILPSHDEGARQSSVSCDQSSQLVSMTLNPVSKEDEGWYWCGVKQGQTYGETTAIYIAVEERGSHHHHHH

SEQ ID NO: 26 - protein, 587 aa, synthetic construct
MRLYLFTLLVTVFSGVSTKSPIFGPQEVSSIEGDSVSITCYYPDTSVNRHTRKYWCRQGASGMCTTLISSNGYLSKEYSGRANLINFPENNTFVINIEQLTQDDTGSYKCGLGTSNRGLSFDVSLEVSQVPELPSQVQLQQSDAELVKPGASVKISCKASGYTFTDHAIHWVKQKPEQGLEWIGYISPGNDDIKYNEKFKGKATLTADTSSSTAYMQLNSLTSEDSAVYFCKVLRRFAYWGQGTLVTVSAAPEPELLYKDLRSSVTFECDLGREVANEAKYLCRMNKETCDVIINTLGKRDPDFEGRILITPKDDNGRFSVLITGLRKEDAGHYQCGAHSSGLPQEGWPIQTWQLFVNEESTIPNRRSVVKGVTGGSVAIACPYNPKESSSLKYWCRWEGDGNGHCPVLVGTQAQVQEEYEGRLALFDQPGNGTYTVILNQLTTEDAGFYWCLTNGDSRWRTTIELQVAEATREPNLEVTPQNATAVLGETFTVSCHYPCKFYSQEKYWCKWSNKGCHILPSHDEGARQSSVSCDQSSQLVSMTLNPVSKEDEGWYWCGVKQGQTYGETTAIYIAVEERGSHHHHHH

SEQ ID NO: 27 - protein, 597 aa, synthetic construct
MRLYLFTLLVTVFSGVSTKSPIFGPQEVSSIEGDSVSITCYYPDTSVNRHTRKYWCRQGASGMCTTLISSNGYLSKEYSGRANLINFPENNTFVINIEQLTQDDTGSYKCGLGTSNRGLSFDVSLEVSQVPELPSQVKLEESGGGLVQAGGSLRLSCADSERTFRIYTMAWFRQAPGKERDFVAAISWSGGSTYYADSVKGRFTISRDNAKNTVYLPMNSLKPDDTAVYYCASGGVLSTGSQSDSEYDFWGQGTQVTVSSAPEPELLYKDLRSSVTFECDLGREVANEAKYLCRMNKETCDVIINTLGKRDPDFEGRILITPKDDNGRFSVLITGLRKEDAGHYQCGAHSSGLPQEGWPIQTWQLFVNEESTIPNRRSVVKGVTGGSVAIACPYNPKESSSLKYWCRWEGDGNGHCPVLVGTQAQVQEEYEGRLALFDQPGNGTYTVILNQLTTEDAGFYWCLTNGDSRWRTTIELQVAEATREPNLEVTPQNATAVLGETFTVSCHYPCKFYSQEKYWCKWSNKGCHILPSHDEGARQSSVSCDQSSQLVSMTLNPVSKEDEGWYWCGVKQGQTYGETTAIYIAVEERGSHHHHHH

SEQ ID NO: 28 - protein, 583 aa, synthetic construct
MRLYLFTLLVTVFSGVSTKSPIFGPQEVSSIEGDSVSITCYYPDTSVNRHTRKYWCRQGASGMCTTLISSNGYLSKEYSGRANLINFPENNTFVINIEQLTQDDTGSYKCGLGTSNRGLSFDVSLEVSQVPELPSDIVLTQSPASLAVSLGQRATISCRASKSVSTSGYSYMHWYQQKPGQPPKLLIYLASNLESGVPARFSGSGSGTDFTLNIHPVEEEDAATYYCQHSRELPRTFGGGTKLEIKAPEPELLYKDLRSSVTFECDLGREVANEAKYLCRMNKETCDVIINTLGKRDPDFEGRILITPKDDNGRFSVLITGLRKEDAGHYQCGAHSSGLPQEGWPIQTWQLFVNEESTIPNRRSVVKGVTGGSVAIACPYNPKESSSLKYWCRWEGDGNGHCPVLVGTQAQVQEEYEGRLALFDQPGNGTYTVILNQLTTEDAGFYWCLTNGDSRWRTTIELQVAEATREPNLEVTPQNATAVLGETFTVSCHYPCKFYSQEKYWCKWSNKGCHILPSHDEGARQSSVSCDQSSQLVSMTLNPVSKEDEGWYWCGVKQGQTYGETTAIYIAVEERGSHHHHHH

SEQ ID NO: 29 - protein, 590 aa, synthetic construct
MRLYLFTLLVTVFSGVSTKSPIFGPQEVSSIEGDSVSITCYYPDTSVNRHTRKYWCRQGASGMCTTLISSNGYLSKEYSGRANLINFPENNTFVINIEQLTQDDTGSYKCGLGTSNRGLSFDVSLEVSQVPELPSEVQLQQSGPELVKPGASVKISCKASGYTFTDYNMWVKQSHGKSLEWIGYIYPYNGGTGYNQKFKSKATLTVDNSSSTAYMELRSLTSEDSAVYYCARNYYGSSWFAYWGQGTLVTVSAAPEPELLYKDLRSSVTFECDLGREVANEAKYLCRMNKETCDVIINTLGKRDPDFEGRILITPKDDNGRFSVLITGLRKEDAGHYQCGAHSSGLPQEGWPIQTWQLFVNEESTIPNRRSVVKGVTGGSVAIACPYNPKESSSLKYWCRWEGDGNGHCPVLVGTQAQVQEEYEGRLALFDQPGNGTYTVILNQLTTEDAGFYWCLTNGDSRWRTTIELQVAEATREPNLEVTPQNATAVLGETFTVSCHYPCKFYSQEKYWCKWSNKGCHILPSHDEGARQSSVSCDQSSQLVSMTLNPVSKEDEGWYWCGVKQGQTYGETTAIYIAVEERGSHHHHHH

SEQ ID NO: 30 - protein, 123 aa, synthetic construct
QVQLVESGGGLAQAGGSLRLSCAASGRTFSMDPMAWFRQPPGKEREFVAAGSSTGRTTYYADSVKGRFTISRDNAKNTVYLQMNSLKPEDTAVYYCAAAPYGANWYRDEYDYWGQGTQVTVSS

SEQ ID NO: 31 - protein, 124 aa, synthetic construct [sequence omitted]

SEQ ID NO: 32 - protein, 123 aa, synthetic construct
QVKLEESGGGLVQAGGSLRLSCAASRLTFSTYHMGWFRQAPGKEREFVAALSWSGGTTYYADSVKGRFGISRDNAKNTVYLQMNSLKPEDTAVYYCASGGVLATMNSDEYDYWGQGTQVTVSS

SEQ ID NO: 33 - protein, 125 aa, synthetic construct
QVKLEESGGGLVQAGGSLRLSCADSERTFRIYTMAWFRQAPGKERDFVAAISWSGGSTYYADSVKGRFTISRDNAKNTVYLPMNSLKPDDTAVYYCASGGVLSTGSQSDSEYDFWGQGTQVTVSS

SEQ ID NO: 34 - protein, 119 aa, synthetic construct
EVQLVQSGAEVKKSGESLKISCKGSGYSFTSYWIGWVRQMPGKGLEWMGIFYPGDSSTRYSPSFQGQVTISADKSVNTAYLQWSSLKASDTAMYYCARRRNWGNAFDIWGQGTMVTVSS

SEQ ID NO: 35 - protein, 108 aa, synthetic construct
EIVLTQSPGTLSLSPGERATLSCRASQSVSSSYLAWYQQKPGQAPRLLIYGASSRATGIPDRFSGSGSGTETTLTISRLEPEDFAVYYCQQYGSSTWTFGQGTKVEIK

SEQ ID NO: 36 - protein, 122 aa, synthetic construct
QVQLVESGGGVVQPGRSLRLSCAASGFSFSNYGMHWVRQAPGKGLEWVALIWYDGSNEDYTDSVKGRFTISRDNSKNTLYLQMNSLRAEDTAVYYCARWGMVRGVIDVFDIWGQGTVVTVSS

SEQ ID NO: 37 - protein, 107 aa, synthetic construct
DIQMTQSPSSVSASVGDRVTITCRASQGISSWLAWYQHKPGKAPKLLIYAASSLQSGVPSRFSGSGSGTDFTLTISSLQPEDFATYYCQQANSFPWTFGQGTKVEIK

SEQ ID NO: 38 - protein, 109 aa, synthetic construct
EIVLTQSPATLSLSPGERATLSCRASQSVGTSIHWYQQKPGQAPRLLIKFASESISGIPARFSGSGSGTDFTLTISSLEPEDFAVYYCQQSNKWPFTFGQGTKLEIKRT

SEQ ID NO: 39 - protein, 106 aa, synthetic construct
EIVLTQSPATLSLSPGERATLSCRASSSVNYMNWYQQKPGQAPRPLIYATSNLASGIPARFSGSGSGTDFTLTISSLEPEDFAVYYCQQWSSRTFGGGTKLEIKRT

SEQ ID NO: 40 - protein, 115 aa, synthetic construct
QVQLQQSDAELVKPGASVKISCKASGYTFTDHAIHWVKQKPEQGLEWIGYISPGNDDIKYNEKFKGKATLTADTSSSTAYMQLNSLTSEDSAVYFCKVLRRFAYWGQGTLVTVSA

SEQ ID NO: 41 - protein, 109 aa, synthetic construct
QAVVTQESALTTSPGETVTLTCRSSNGAVTSRNYANWVQEKPDHLFTGLIGGTNNRAPGVPARFSGSLIGDKAALSITGAQTEDEAIYFCALWYSNRWVFGGGTKLTVL

SEQ ID NO: 42 - protein, 118 aa, synthetic construct
EVQLQQSGPELVKPGASVKISCKASGYTFTDYNMWVKQSHGKSLEWIGYIYPYNGGTGYNQKFKSKATLTVDNSSSTAYMELRSLTSEDSAVYYCARNYYGSSWFAYWGQGTLVTVSA

SEQ ID NO: 43 - protein, 111 aa, synthetic construct
DIVLTQSPASLAVSLGQRATISCRASKSVSTSGYSYMHWYQQKPGQPPKLLIYLASNLESGVPARFSGSGSGTDFTLNIHPVEEEDAATYYCQHSRELPRTFGGGTKLEIK

SEQ ID NO: 44 - protein, 141 aa, synthetic construct
QVQLVESGGGLVQAGGSLRLSCAASGLTFSRYVMGWFRQAPGKEREFVAAITWGGTPNYADSVKGRFTISRDNSKNTQYLQMNSLKPEDTAVYYCAAGLGWDSRYSQSYNYWGQGTQVTVSSGSEQKLISEEDLNHHHHHH

SEQ ID NO: 45 - protein, 114 aa, synthetic construct
QMQLVESGGGVVQPGRSLRLSCEASGFSFNSYGMHWVRQAPGKGLEWVSVIWASGNKKYYIESVEGRFTISRDNSKNTLYLQMNSLRAEDTAVYYCARANFDYWGQGTLVTVSS

SEQ ID NO: 46 - protein, 108 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWSQFTFGPGTKVDIK

SEQ ID NO: 47 - protein, 116 aa, synthetic construct
QVQLVESGGGVVQPGRSLRLSCAASGFTFNKYGMHWVRQAPGKGLEWVAVIWYDGTNKYYADSMKGRFTISRDNSKNMLYLQMNSLRAEDTAVYYCARDPPTANYWGQGTLVTVSS

SEQ ID NO: 48 - protein, 107 aa, synthetic construct
DIQMTQSPSSLSASVGDRVTITCRASQGISSWLAWYQQKPEKAPKSLIYAASSLQSGVPSRFSGSGSGTDFTLTISSLQPEDFATYYCQQYKSYPVTFGGGTKVEIK

SEQ ID NO: 49-50 - skipped (2 entries)

SEQ ID NO: 51 - protein, 127 aa, synthetic construct
QVQLVESGGGLVQAGGSLRLSCTDSGRTFSVKPMGWFRQAPGMEREFVAAASFTGVSTFYADSVKDRFTIFRDKDKNTMDLQINSLKPEDTGAYYCAGTTRTLWGSKWRDVLEYEYWGQGTQVTVSS

SEQ ID NO: 52 - protein, 216 aa, synthetic construct
DVLMTQTPLSLPVSLGDQASISCRSSQTIVHSNGKIYLEWYLQKPGQSPKLLIYRVSKRFSGVPDRFSGSGSGTDFTLKISRVEAEDLGVYYCFQGSHVPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNR

SEQ ID NO: 53 - protein, 215 aa, synthetic construct
EVKLVESGGGLVAPGGSLKLSCAASGFTFSSYPMSWVRQTPEKRLEWVAYINNGGGNPYYPDTVKGRFTISRDNAKNTLYLQMSSLKSEDTAIYYCIRQYYGFDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVP

SEQ ID NO: 54-56 - skipped (3 entries)

SEQ ID NO: 57 - protein, 112 aa, synthetic construct
DVVMTQTPLSLPVSLGDQASMSCRSSQSLVHNNGDTYLHWYLQKPGRSPKLLLHKVSNRLSGVPDRFSGSGSGTDFTLKISRVETEDLGVYFCSQSTHVPWTFGGGTKLEIK

SEQ ID NO: 58 - protein, 125 aa, synthetic construct
QVKLEESGGGLVQAGGSLRLSCAASIRSFSYRNMGWFRQPPGKEREFVAAITWDGGSTRYADSVKGRFTVSRDNAKKTVYLQMNSLKPEDAAVYYCAAGFGHTLATSSDEYDYWWGQGTQVTVSS

SEQ ID NO: 59 - protein, 125 aa, synthetic construct
QVKLEESGGGLVQAGGSLRLSCAASGRTFNTLSMGWFRQAPGKEREFVAAVSRSGGSTYYADSVKGRFTISRDNAKNTVYLQMNSLKPEDTAVYYCAAAATKSNTTAYRLSFDYWGQGTQVTVSS

SEQ ID NO: 60 - protein, 128 aa, synthetic construct
QVKLEESGGGLVQPGGSLRLSCAASGRTLSSYIVAWFRQAPGKEREFVAGISRRGGNSAYVESVKGRFTISRDNAKNTVYLQMNSLKPEDTAVYYCAADGSVAGWGRRSVSVSSYDYWGQGTQVTVSS

SEQ ID NO: 61 - protein, 124 aa, synthetic construct [sequence omitted]

SEQ ID NO: 62 - protein, 124 aa, synthetic construct
QVQLEESGGGLVQAGGSLRLSCAASERTFSRYPVAWFRQAPGAEREFVAVISSTGTSTYYADSVKGRFTISRDNAKVTVYLQMNNLKREDTAVYFCAVNSQRTRLQDPNEYDYWGQGTQVTVSS

SEQ ID NO: 63 - protein, 123 aa, synthetic construct
QVKLEESGGGLVQPGGSLRLSCAASGGTFTNYAMAWFRQTPGNDREFVGIISQKGGRTYYADSVKGRFSVSRDNAKNTAYLQMNSLKPEDTAVYYCAAGDSSYYYTRSRYDIWGQGTQVTVSS

SEQ ID NO: 64 - protein, 135 aa, synthetic construct
QVQLVESGGGLVQAGDSLRLSCAASGGTFSSYAVGWFRQAPGKERQFVAAISWSGRSTEYADSVKGRFTISRDNAKNTVYLQMNSLQPDDTGVYYCAADWSHPENKAELLRLRLWVLSAESSDYWGQGTQVTVSS

SEQ ID NO: 65 - protein, 132 aa, synthetic construct
QVQLVESGGGLVQPGGSLRLSCAASGFTLDSYAIGWFRQAPGKEHEGISCISSNGGSTYYTDSVKGRFTISRDNAKNTVYLQMNSLESEDSAVYYCATVRRCSSLDMALGALATRTKGYDYWGQGTQVTVSS

SEQ ID NO: 66 - protein, 135 aa, synthetic construct
QVQLVESGGGLVQAGGSLRLSCAASGRTFSSYAVGWFRQAPGKEREFVAAISWSGGYTDYADSVKGRFTISRDNAKNTVYLQMNNLKPEDTAVYYCAADWSHPENKAELLRLRLWVLSAESSDDWGQGTQVTVSS

SEQ ID NO: 67 - protein, 122 aa, synthetic construct
QVKLEESGGGLVQAGGSLRLSCAASGRTFTNYAMAWFRQASGKEREFVAIISQSGGRTYYGDSVKGRFTISRDNAKNTVYLQLNSLQPEDTGVYYCAAGDSPYYSRSRYDLWGPGTQVTVSS

SEQ ID NO: 68 - protein, 131 aa, synthetic construct
QVKLEESGGGLVQAGGSLRLSCAASEGAFSDALSRHAAGWFRQAPGKEREFVAAISWNGANTDYKNSVNNRFTISRDTSKNTVYLQMNSLKPEDTAVYYCAANEPSWISRIYYRGLRYDLWGQGTQVTVSS

SEQ ID NO: 69 - protein, 127 aa, synthetic construct
QVKLEESGGGLVQPGGSLRLSCAASGFTLDYYTIGWFRQAPGKEREGVACISSDDRTYYVDSVKGRFAISRDNVKNTVYLQMNNLKPEDTAYYCASKESVFLIATMKGCAPGHDYYWGQGTQVTVSS

SEQ ID NO: 70 - protein, 119 aa, synthetic construct
QVQLVESGGGLVQAGGSLRLSCAASGSFFMSNVMAWYRQAPGKARELIAAIRGGDMSTVYDDSVKGRFTITRDDDKNILYLQMNDLKPEDTAMYYCKASGSSWGQGTQVTVSSHHHHHH

SEQ ID NO: 71 - protein, 121 aa, synthetic construct
QVQLVESGGGLVQPGGSLRLSCAASGNIFSINTMGWYRQAPGKQLELVAAITSGGTTSYTDSVEGRFTISRDNAKNAVYLQMNSLKAEDTAVYYCNTVKVVGGRLDNPDYWGQGTQVTVSS

SEQ ID NO: 72 - protein, 127 aa, synthetic construct
QVKLEESGGGLVQPGGSLRLSCAASGRTASGYGMGWFRQAPGKEREFVAAISRSGAGTLNADFVKGRFTISRDNAKNTVYLQMNSLKPEDTAVYYCVARPTKVDRDYATRREMYNYWGQGTQVTVSS

SEQ ID NO: 73 - protein, 125 aa, synthetic construct
QVKLEESGGGSVQAGGSLRLSCAASGRDFSTLAMGWFRQAPGKEREFVATINWSGGTTHYADSVKGRFTISRDNAKNTVYLQMGSLKPEDTAVYYCGRSKYAAGALTRAYDYNYWGQGTQVTVSS

SEQ ID NO: 74 - protein, 125 aa, synthetic construct
QVKLEESGGGLVQAGGSLRLSCSASGSIFSINDMGWYRRAPGKRRELVAAITSGGIPNYADSVKGGRFTISRDNAKNTGYLQMNSLKPEDTAVYYCAAQFGTVAAALRRHEYDYWGQGTQVTVSS

SEQ ID NO: 75 - protein, 122 aa, synthetic construct
QVKLEESGGGLVQAGGSLRLSCSASGRTFSSGVMGWFRQAPGKQRELVAAITTGGSTSYTDSVKGGRFTISRDNAKNTVYLQMNSLKPEDTAVYYCNSVAVVGGVIKSPDYWGQGTQVTVSS

SEQ ID NO: 76 - protein, 123 aa, synthetic construct
QVQLVESGGGSVQAGGSLRLSCAASGLSRYAMAWFRQGTGKEREFVASTNWSSGNTPYADSVKGGRFIISRDNAKNTVYLQMNSLKPGDTAIYYCAARKLDVPSRYSQHYDYWGQGTQVTVSS

SEQ ID NO: 77 - protein, 122 aa, synthetic construct
QVQLVESGGDLVQAGGSLRLSCAASGSISRISTMGWYRQAPGKQRELVATISTGGTTNYAESVKGGRFTVSRDNAKNTMYLQMNSLKPEDTAVYYCAAGWKVVRGSLEYEYSGQGTQVTVSS

SEQ ID NO: 78 - protein, 126 aa, synthetic construct
DVQLVESGGGLVQPGGSLRLSCAASGSIFSIYAMGWYRQAPGKQRELVASISSGGGTNYADSVKGRFTISGDNAKNTVYLQMNSLKPEDTAVYYCKREDYSAYAPPSGSRGRGTQVTVSSHHHHHH

SEQ ID NO: 79 - protein, 126 aa, synthetic construct; identical to SEQ ID NO: 78

SEQ ID NO: 80 - protein, 119 aa, synthetic construct
DVQLVESGGGLVQPGGSLRLSCAASESILSFNHMAWYRQGPGEQRELVAVITREGSTDYADSVKGRFTISRDNAKNMVYLLMSNLRPEDTAVYYCNRGISNPWGQGTQVTVSSHHHHHH

SEQ ID NO: 81 - protein, 265 aa, synthetic construct
METDTLLLWVLLLWVPGSTGDDIQMTQSPASLAVPLLLWISGAYGDIVLAQSPDSLAVSPGERATIHCKSSQTLLYSSNNRHSIAWYQQRPGQPPKLLLYWASMRLSGVPDRFSGSGSGTDFTLTINNLQAEDVAIYYCHQYSSHPPTFGHGTRVELRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 82 - protein, 252 aa, synthetic construct
METDTLLLWVLLLWVPGSTGDQVQLVQSGGGVFKPGGSLRLSCEASGFTFTEYYMTWVRQAPGKGLEWLAYISKNGEYSKYSPSSNGRFTISRDNAKNSVFLQLDRLSADDTAVYYCARADGLTYFSELLQYIFDLWGQGARVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDK

SEQ ID NO: 83 - protein, 236 aa, synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 84 - protein, 227 aa, synthetic construct
MNSLIKENMRMKVVLEGSVNGHQFKCTGEGEGNPYMGTQTMRIKVIEGGPLPFAFDILATSFMYGSRTFIKYPKGIPDFFKQSFPEGFTWERVTRYEDGGVITVMQDTSLEDGCLVYHAQVRGVNFPSNGAVMQKKTKGWEPNTEMMYPADGGLRGYTHMALKVDGGGHLSCSFVTTYRSKKTVGNIKMPGIHAVDHRLERLEESDNEMFVVQREHAVAKFAGLGGG

SEQ ID NO: 85 - protein, 236 aa, synthetic construct
MVSKGEENNMAVIKEFMRFKVRMEGSVNGHEFEIEGEGEGRPYEGTQTAKLKVTKGGPLPFAWDILSPQFCYGSKAYVKHPTGIPDYFKLSFPEGFKWERVMNFEDGGVVTVAQDSSLQDGEFIYKVKLRGTNFPSDGPVMQKKTMGWEASSERMYPEDGALKGEIKMRLKLKDGGHYSAETKTTYKAKKPVQLPGAYIAGEKIDITSHNEDYTIVELYERAEGRHSTGGMDELYK

SEQ ID NO: 86 - protein, 225 aa, synthetic construct
MASSEDVIKEFMRFKVRMEGSVNGHEFEIEGEGEGRPYEGTQTAKLKVTKGGPLPFAWDILSPQFCYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKMRLKLKDGGHYDAEVKTTYMAKKPVQLPGAYKTDIKLDITSHNEDYTIVELYERAEGRHSTGA

SEQ ID NO: 87 - protein, 236 aa, synthetic construct
MVSKGEENNMAIIKEFMRFKVRMEGSVNGHEFEIEGEGEGRPYEGTQTAKLKVTKGGPLPFAWDILTPNFTYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKMRLKLKDGGHYDAEVKTTYKAKKPVQLPGAYIVGIKLDITSHNEDYTIVELYERAEGRHSTGGMDELYK

SEQ ID NO: 88 - protein, 225 aa, synthetic construct
MASSEDVIKEFMRFKVRMEGSVNGHEFEIEGEGEGRPYEGTQTAKLKVTKGGPLPFAWDILSPQFMWGSKAYVKHPADIPDYLKLSFPEGFKWERVMNFEDGGVVTVTQDSSLQDGEFIYKVKLRGTNFPSDGPVMQKKTMGWAATTERMYPEDGALKGEIKMRLKLKDGGHYDAEVKTTYMAKKPVQLPGAYKIDGKLDITSHNEDYTIVEQYERAEGRHSTGA

SEQ ID NO: 89 - protein, 238 aa, synthetic construct
MSKGEELFTGVVPILVELDGDVNGHKFSVRGEGEGDATNGKLTLKFICTTGKLPVPWPTLVTTLTYGVLCFSRYPDHMKRHDFFKSAMPEGYVQERTISFKDDGTYKTRAEVKFEGDTLVNRIELKGIDFKEDGNILGHKLEYNFNSHNVYITADKQKNGIKAYFKIRHNVEDGSVQLADHYQQNTPIGDGPVLLPDNHYLSTQSVLSKDPNEKRDHMVLLEDVTAAGITHGMDELYK

SEQ ID NO: 90 - protein, 244 aa, synthetic construct [sequence omitted]

SEQ ID NO: 91 - protein, 106 aa, synthetic construct
MEKSFVITDPRLPDNPIIFASDGFLELTEYSREEILGRNGRFLQGPETDQATVQKIRDAIRDQREITVQLINYTKSGKKFWNLLHLQPMRDQKGELQYFIGVQLDG

SEQ ID NO: 92 - protein, 371 aa, synthetic construct
KKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQP

SEQ ID NO: 93 - protein, 463 aa, synthetic construct
KEQKDYLLTMQKSVTVQEGLCVSVLCSFSYPQNGWTASDPVHGYWFRAGDHVSRNIPVATNNPARAVQEETRDRFHLLGDPQNKDCTLSIRDTRESDAGTYVFCVERGNMKWNYKYDQLSVNVTASQDLLSRYRLEVPESVTVQEGLCVSVPCSVLYPHYNWTASSPVYGSWFKEGADIPWDIPVATNTPSGKVQEDTHGRFLLLGDPQTNNCSLSIRDARKGDSGKYYFQVERGSRKWNYIYDKLSVHVTALTHMPTFSIPGTLESGHPRNLTCSVPWACEQGTPPTITWMGASVSSLDPTITRSSMLSLIPQPQDHGTSLTCQVTLPGAGVTMTRAVRLNISYPPQNLTMTVFQGDGTASTTLRNGSALSVLEGQSLHLVCAVDSNPPARLSWTWGSLTLSPSQSSNLGVLELPRVHVKDEGEFTCRAQNPLGSQHISLSLSLQNEYTGKMRPISGVTLGA

SEQ ID NO: 94 - protein, 244 aa, synthetic construct
FVRTKIDTTENLLNTEVHSSPAQRWSMQVPPEVSAEAGDAAVLPCTFTHPHRHYDGPLTAIWRAGEPYAGPQVFRCAAARGSELCQTALSLHGRFRLLGNPRRNDLSLRVERLALADDRRYFCRVEFAGDVHDRYESRHGVRLHVTAAPRIVNISVLPSPAHAFRALCTAEGEPPPALAWSGPALGNSLAAVRSPREGHGHLVTAELPALTHDGRYTCTAANSLGRSEASVYLFRFHGASGAST

SEQ ID NO: 95 - protein, 112 aa, synthetic construct
MGHHHHHHVVPKTATSTETKTITRIIHYVDKVTNQNVKEDVVQPVTLSRTKTENKVTGVVTYGEWTTGNWDEVISGKIDKYKDPDIPTVESQEVTSDSSDKEITVRYDRLST

SEQ ID NO: 96 - protein, 153 aa, synthetic construct
MSGSHHHHHHSSGIEGRGRLIKHMTAEAGITGTWYNQLGSTLIVTAGADGALTGTYESAVGNAEGSYVLTGRYDSAPATDGSGTALGWTVAWKNNYRNAHSASTWSGQYVGGAEARINTQVLTTSGTTEANAWKSTLVGHDTFTKVKPSAASI

SEQ ID NO: 97 - protein, 128 aa, synthetic construct
AECSVDIQGNDQMQFNTNAITVDKSCKQFTVNLSHPGNLPKNVMGHNWVLSTAADMQGVVTDGMASGLDKDYLKPDDSRVIAHTKLIGSGEKDSVTFDVSKLKEGEQYMFFDTFPGHSALIKGTLTLK

SEQ ID NO: 98 - protein, 141 aa, synthetic construct
MRGSTRDQNGTWEMESNENFEGYMKALDIDFATRKIAVRLTQTKVIDQDGDNFKTKTTSTFRNYDVDFTVGVEFDEYTKSLDNRHVKALVTWEGDVLVCVQKGEKENRGWKQWIEGDKLYLELTCGDQVCRQVFKKKLVPR

SEQ ID NO: 99 - protein, 164 aa, synthetic construct
MRGSHHHHHHTDPAYVPAPGYQPTYNPTLPYKRPIPGGLSVGMSVYIQGMAKENMRRFHVNFAVGQDDGADVAFHFNPRFDGWDKVVFNTMQSGQWGKEEKKKSMPFQKGKHFELVFMVMPEHYKVVVNGNSFYEYGHRLPVQMVTHLQVDGDLELQSINFLGG

SEQ ID NO: 100 - protein, 118 aa, synthetic construct
METVAYADFARLEMRVGKIVEVKRHENADKLYIVQVDVGQKTLQTVTSLVPYYSEEELMGKTVVVLCNLQKAKMRGETSECMLLCAETDDGSESVLLTPERMMPAGVRVVLDHHHHHH

SEQ ID NO: 101 - protein, 138 aa, synthetic construct
MRGSAFNGKWETESQEGYEPFCKLIGIPDDVIAKGRDFKLVTEIVQNGDDFTWTQYYPNNHVVTNKFIVGKESDMETVGGKKFKGIVSMEGGKLTISFPKYQQTTEISGGKLVETSTASGAQGTAVLVRTSKKVLVPR

SEQ ID NO: 102 - protein, 111 aa, synthetic construct
AWVDQTPRTITKETGESLTIKCVLKDHSCGLSSTTWYRTQLGSTNEKTISIGGRYDETVDKGSKSFSLRISDLRVEDSGTYKCQADYSPSCYSYPSLESAVEGAGTVLTVK

SEQ ID NO: 103 - protein, 164 aa, synthetic construct
GSSGSSGMMLSLNNLQNIIYNPVIPYVGTIPDQLDPGTLIVICGHVPSDADRFQVDLQNGSSVKPRADVAFHFNPRFKRAGCIVCNTLINEKWGREEITYDTPFKREKSFEIVIMVLKDKFQVAVNGKHTLLYGHRIGPEKIDTLGIYGKVNIHSIGFSGPSSG

SEQ ID NO: 104 - protein, 162 aa, synthetic construct
LIVTQTMKGLDIQKVAGTWYSLAMAASDISLLDAQSAPLRVYVEELKPTPEGDLEILLQKWENGECAQKKIIAEKTKIPAVFKIDALNENKVLVLDTDYKKYLLFCMENSAEPEQSLACQCLVRTPEVDDEALEKFDKALKALPMHIRLSFNPTQLEEQCHI

SEQ ID NO: 105 - protein, 176 aa, synthetic construct
VVSFIGSTENDVGPSQGSYSSTHAMDNLPFVYNTGYNIGYQNANVWRIGGGFCVGLDGKVDLPVVGSLDGQSIYGLTEEVGLLIWMGDTNYSRGTAMSGNSWENVFSGWCVGNYLSTQGLSVHVRPVILKRNSSAQYSVQKTSIGSIRMRPYNGSSAGSVQTTVNFSLNPFTLNDT

SEQ ID NO: 106 - protein, 122 aa, synthetic construct
MIEPIKRTQVVTQTIHYRYEDGAVAHDDHVVSLIFTQSGKRDLTNGKEIWDSKWSLTQTFEALPSPVIIGYTADKPMVGPDEVTVDSKNFLDKQNREETVIYSANTITQNKKDGLEHHHHHH

SEQ ID NO: 107 - protein, 107 aa, synthetic construct
THATSTETIHYVNEDGDQVFEDGGGKLDFTRTVTIDDVTNEVVEYGEWTPVTDDEFAAVTSPDKDGYTPDTSEVAAQKPDMTDGPDGTVKDVEVTVTYTANPAVATI

SEQ ID NO: 108 - protein, 308 aa, synthetic construct
GPIIVEPHVTAVWGKNVSLKCLIEVNETITQISWEKIHGKSSQTVAVHHPQYGFSVQGEYQGRVLFKNYSLNDATITLHNIGFSDSGKYICKAVTFPLGNAQSSTTVTVLVEPTVSLIKGPDSLIDGGNETVAAICIAATGKPVAHIDWEGDLGEMESTTTSFPNETATIISQYKLFPTRFARGRRITCVVKHPALEKDIRYSFILDIQYAPEVSVTGYDGNWFVGRKGVNLKCNADANPPPFKSVWSRLDGQWPDGLLASDNTLHFVHPLTFNYSGVYICKVTNSLGQRSDQKVIYISDPPHHHHHH

SEQ ID NO: 109 - protein, 209 aa, synthetic construct
VKPLQVEPPEPVVAVALGASRQLTCRLACADRGASVQWRGLDTSLGAVQSDTGRSVLTVRNASLSAAGTRVCVGSCGGRTFQHTVQLLVYAFPNQLTVSPAALVPGDPEVACTAHKVTPVDPNALSFSLLVGGQELEGAQALGPEVQEEEEEPQGDEDVLFRVTERWRLPPLGTPVPPALYCQATMRLPGLELSHRQAIPVLHSPTSPE

SEQ ID NO: 110 - protein, 75 aa, synthetic construct
ESLQERADEATTQKQSGEDNQDLAISFAGNGLSALRTSGSQARATCYCRTGRCATRESLSGVCEISGRLYRLCCR

SEQ ID NO: 111 - protein, 32 aa, synthetic construct
AFTCHCRRSCYSTEYSYGTCTVMGINHRFCCL

SEQ ID NO: 112 - protein, 151 aa, synthetic construct
NSSASSAQVTGTLLGTGKTNTTQMPALYTWQHQIYNVNFIPSSSGTLTCQAGTILVWKNGRETQYALECRVSIHHSSGSINESQWGQQSQVGFGTACGNKKCRFTGFEISLRIPPNAQTYPLSSGDLKGSFSLTNKEVNWSASIYVPAIAK

SEQ ID NO: 113 - protein, 184 aa, synthetic construct
MQTAYVKYVDDTTGETLRQDDLHGYTDETIPYSTAEGIKKYEGDGYVLVSDGFKPGTKFGVGTPTYEVHFKHGMTHTDATDKNAEQKTVTETIHYVDENNQTVQPDSTTAVTFKRGYTTDNVTGKVVSYDPWTVDGNQADSKTFAAVPSPAVEGYTPNHQQINEFTVTPDSKDIVKTVVYVGDP

SEQ ID NO: 114 - protein, 596 aa, synthetic construct
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNGSEVQLVESGGGLVQAGGSLKLSCAASGRTYAMGWFRQAPGKEREFVAHINALGTRTYYSDSVKGRFTISRDNAKNTEYLEMNNLKPEDTAVYYCTAQGQWRAAPVAVAAEYEFWGQGTQVTVSSGKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGSSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERGSHHHHHH

SEQ ID NO: 115 - protein, 597 aa, synthetic construct
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNGSEVQLVESGGGLVQPGGSLRLSCAVSISIFDIYAMDWYRQAPGKQRDLVATSFRDGSTNYADSVKGRFTISRDNAKNTLYLQMNSLKPEDTAVYLCHVSLYRDPLGVAGGMGVYWGKGALVTVSSSGKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGSSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERGSHHHHH

SEQ ID NO: 116 - protein, 122 aa, synthetic construct
EVQLVESGGGLVQAGGSLKLSCAASGRTYAMGWFRQAPGKEREFVAHINALGTRTYYSDSVKGRFTISRDNAKNTEYLEMNNLKPEDTAVYYCTAQGQWRAAPVAVAAEYEFWGQGTQVTVS

SEQ ID NO: 117 - protein, 124 aa, synthetic construct
EVQLVESGGGLVQPGGSLRLSCAVSISIFDIYAMDWYRQAPGKQRDLVATSFRDGSTNYADSVKGRFTISRDNAKNTLYLQMNSLKPEDTAVYLCHVSLYRDPLGVAGGMGVYWGKGALVTVSS

SEQ ID NO: 118 - protein, 127 aa, synthetic construct
EVQLVESGGGLVQPGGSLRLSCAATGFTLENKAIGWFRQTPGSEREGVLCISKSGSWTYYTDSMRGRFTISRDNAENTVYLQMDSLKPEDTAVYYCATTTAGGGLCWDGTTFSRLASSWGQGTQVTV

(19) United States
(12) Patent Application Publication
(10) Pub. No.: US 2024/0425570 A1
(43) Pub. Date: Dec. 26, 2024

(54) CHIMERIC SECRETORY COMPONENT POLYPEPTIDES AND USES THEREOF

(71) Applicant: THE BOARD OF TRUSTEES OF THE UNIVERSITY OF ILLINOIS, Urbana, IL (US)

(72) Inventors: Beth M. Stadtmueller, Urbana, IL (US); Sonya Kumar Bharathkar, Urbana, IL (US)

(73) Assignee: THE BOARD OF TRUSTEES OF THE UNIVERSITY OF ILLINOIS, Urbana, IL (US)

(21) Appl. No.: 18/692,514

(22) PCT Filed: Sep. 16, 2022
(86) PCT No.: PCT/US2022/076548

§ 371 Date: Mar. 15, 2024

Related U.S. Application Data
(60) Provisional application No. 63/245,342, filed on Sep. 17, 2021.

(51) Int. Cl. C07K 16/12; C07K 16/10

(52) U.S. Cl. CPC C07K 16/1282; C07K 16/1018; C07K 2317/52; C07K 2317/55; C07K 2317/569; C07K 2317/76

(57) ABSTRACT

Recombinant polypeptides that include a chimeric secretory component (cSC) protein in which the D2 domain of secretory component is modified to confer one or more non-native properties to the polypeptide are described. The D2 domain can be modified, for example, to confer specific binding to a target molecule, such as by replacing the D2 domain with a single domain antibody or by modifying the D2 domain by replacement of complementarity determining region (CDR)-like loops with CDR sequences from a single domain antibody. The D2 domain can also be modified to enable fluorometric or colorimetric detection of the recombinant polypeptide. Methods of using the recombinant polypeptides, such as for treating or inhibiting a microbial infection, are also described. In some examples of these methods, the D2 domain of the recombinant polypeptide is modified to confer specific binding to a microbial antigen.

### CROSS REFERENCE TO RELATED APPLICATIONS

This application claims the benefit of U.S. Provisional Application No. 63/245,342, filed Sep. 17, 2021, which is herein incorporated by reference in its entirety.

### FIELD

This disclosure concerns recombinant polypeptides that include a chimeric secretory component (cSC) protein having a modified D2 domain that confers one or more non-native properties to the polypeptide. This disclosure further concerns methods of using the recombinant polypeptides, such as for treating a microbial infection.

### BACKGROUND

Secretory component (SC) is one constituent of secretory immunoglobulin A (SIgA) and M (SIgM), and includes the extracellular part of the polymeric immunoglobulin receptor (pIgR), which is made up to five Ig-like domains (D1-D5). Mediated by the joining-chain (JC), polymeric IgA and IgM bind to pIgR on the basolateral surface of epithelial cells and are taken up into cells via transcytosis. The receptor-immunoglobulin complex passes through cellular compartments before being secreted on the luminal surface of epithelial cells. Following proteolysis of the pIgR ectodomain to form SC, complexes of SC and polymeric IgA or polymeric IgM are able to diffuse freely throughout the lumen. SC has a number of biological functions, including for enhancing stability of secretory immunoglobulins (SIg), such as by promoting resistance to proteolytic degradation by host and bacterial enzymes in the intestinal lumen (Duc et al., J Biol Chem 285:953-960, 2010; Crottet and Corthesy, J Immunol 161:5445-5453, 1998); aiding in localization of SIg in the mucus layer (Huang et al., J Proteom Res 14:1335-1349, 2015; Pierce-Cretel et al., Eur J Biochem 125:383-388, 1982); promoting intralumenal sequestration of bacteria (Mathias and Corthesy, J Biol Chem 286:17239-17247, 2011); and performing homeostatic functions in the epithelium (Turula and Wobus, Viruses 10(5):237, 2018).

### SUMMARY

Described herein are recombinant polypeptides that include a chimeric secretory component (cSC) protein in which the D2 domain of secretory component is modified to confer one or more non-native properties to the polypeptide. For example, the D2 domain can be modified to confer specific binding to a target molecule, such as by replacing the D2 domain with a single domain antibody (sdAb) or by modifying the D2 domain by insertion of complementarity determining region (CDR) sequences from a sdAb. The D2 domain can also be modified to enable fluorometric or colorimetric detection of the recombinant polypeptide, such as by substitution of the D2 domain with a fluorescent protein.

Provided herein are recombinant polypeptides that include a cSC protein. In some implementations, the D2 domain of the cSC includes at least one modification that confers specific binding to a target molecule or enables fluorometric or colorimetric detection of the recombinant polypeptide. In some examples, the at least one modification of the D2 domain includes substitution of CDR-like loops of the D2 domain with CDRs of a single-domain antibody, a variable heavy (VH) domain or a variable light (VL) domain; substitution of the D2 domain with a single-domain antibody, a VH domain or a VL domain; substitution of the D2 domain with a first member of a specific binding pair; substitution of the D2 domain with an endolysin; substitution of the D2 domain with a fluorescent protein; or substitution of the D2 domain with Azurin for colorimetric detection.

In some implementations of the recombinant polypeptide, the target molecule is an antigen, such as a bacterial antigen or a viral antigen. In other implementations, the target molecule is a second member of a specific binding pair. In yet other implementations, the target molecule includes a bacterial peptidoglycan.

In some implementations, the recombinant polypeptide further includes polymeric IgA or polymeric IgM. In some examples, the polymeric IgA specifically binds a mucosal antigen, such as a pathogen protein or carbohydrate through its antigen binding fragments (e.g., Fabs).

Also provided herein are methods of treating or inhibiting a bacterial infection in a subject by administering to the subject a therapeutically or prophylactically effective amount of a recombinant polypeptide disclosed herein, such as a recombinant polypeptide having a D2 domain modified to specifically bind a bacterial antigen. In some implementations, the bacterial infection is caused by Clostridium difficile, Salmonella enterica, Salmonella Tm, Streptococcus pneumoniae, Staphylococcus aureus, Listeria monocytogenes, or Campylobacter Jejuni.

Further provided herein are methods of treating or inhibiting a viral infection in a subject by administering to the subject a therapeutically or prophylactically effective amount of a recombinant polypeptide disclosed herein, such as a recombinant polypeptide having a D2 domain modified to specifically bind a viral antigen. In some implementations, the viral infection is caused by HIV-1, SARS-CoV-2, influenza virus or norovirus.

The foregoing and other objects and features of the disclosure will become more apparent from the following detailed description, which proceeds with reference to the accompanying figures.

### BRIEF DESCRIPTION OF THE DRAWINGS

FIG. 1: Schematic showing monomeric immunoglobulin (mIg), polymeric immunoglobulin (pIg), and secretory immunoglobulin (SIg) components in mammals as well as their assembly in plasma cells, their transport to the mucosa by pIg receptor (pIgR) and a subset of their mucosal effector functions, which includes regulation of mucosal homeostasis by physical mechanisms like agglutination or enchained growth of the pathogen.

FIG. 2: Structures of dimeric IgA (dIgA, PDB code 7JG1) and SIgA (upper left, PDB code 7JG2) without Fabs; complex components and associated angles of bend and tilt are indicated. Also shown are SIgA with Fabs modeled in possible positions (upper right) and higher order structures of human SIgA and SIgM lacking Fabs (bottom left).

FIGS. 3A-3B: (FIG. 3A) Structures of secretory component (SC) (PDB code 5D4K) and SC bound to dIgA (PDB code 7JG2) along with schematic showing chimeric (c) SC design. (FIG. 3B) Schematic showing representative monomeric antibodies that can be combined with joining chain (JC) and cSC to generate a library of unique cSIgA.

FIGS. 4A-4B: Analytical size exclusion chromatography (SEC) data showing binding of cSC and cFcα to C. difficile toxin fragment TXA1. The cSC^{20.1} is cSC, in which D2 is replaced by sdAb 20.1; see Table 1, and the cSFcα^{20.1} is cSC^{20.1} bound to dimeric Fcα, a dimeric IgA lacking Fabs. (FIG. 4A) SEC elution profiles for cSC^{20.1}, TcdA toxin fragment, TXA1 and the TXA1-cSC^{20.1} complex along with SDS PAGE of a representative fraction from each peak. (FIG. 4B) SEC elution profiles for cSFcα^{20.1}, TXA1 and TXA1-cSFcα^{20.1} complex along with SDS-PAGE of a representative fraction from each peak. TXA1 failed to bind wildtype SC in control experiments. In SEC panels, the identities of proteins and complexes are defined in the key.

FIG. 5: Schematic of experimental approach using cSC, cSFcα and associated bi-specific cSIgA, in Vero cell cytotoxicity assays (containing C. difficile toxin) and C. difficile growth neutralization assays.

FIG. 6: Neutralization potency of monospecific cSC and cSIgAs in Vero cell cytotoxicity assays containing 50 pM TcdA. Neutralization curves demonstrate that cSC^{A20.1}, cFcα and cSIgA variants, in which cSC^{A20.1} are in complex with dimeric Fcα (FcA2) or dimeric IgAs, can neutralize the cytotoxic effects of C. difficile toxin TcdA. In the absence of cSC, 50 pM TcdA causes ˜100% Vero-cell death that can be prevented by cSC^{A20.1} and its complexes. The positive control is A20.1-Fcα2, which is the sdAb A20.1 fused to the IgA-Fc. The negative control is wildtype SC.

FIG. 7: Neutralization potency of bispecific cSIgAs in Vero cell cytotoxicity assays containing 50 pM TcdA. The bispecific cSIgA PA41-S^{A20.1}IgA2, which incorporates cSC^{A20.1} and antibody PA41 shows enhanced neutralization of TcdA compared to proteins and complexes that incorporate cSC^{A20.1} or PA41 alone, indicating a synergistic effect when the two are combined in a bispecific cSIgA. The cSIgA (PA41-S^{A20.1}IgA2) is capable of binding different epitopes of TcdA with Fabs (PA41) and cSC (sdAb-A20.1).

FIG. 8: Neutralization potency of cSC and cSIgAs in Vero cell cytotoxicity assays containing 50 pM TcdA and 4 pM TcdB. Fifty pM TcdA and 4 pM TcdB kill ˜100% of Vero cells in unsupplemented culture media. The addition of proteins containing the PA41 Fab are capable of neutralizing both TcdA and TcdB, while cSC^{A20.1} can neutralize TcdA only. The bispecific cSIgA PA41-S^{A20.1}IgA2, which incorporates cSC^{A20.1} and antibody PA41, shows enhanced neutralization of TcdA and TcdB compared to proteins and complexes that incorporate cSC^{A20.1} or PA41 alone, indicating a synergistic effect against two toxins when combined in a bispecific cSIgA.

FIGS. 9A-9C: (FIG. 9A) Schematic and SEC elution profile of the cSC^{mCherry}, which has D2 substituted by mCherry (indicated as a star in the schematic). (FIG. 9B) The absorption profile of cSC^{mCherry} from 500 to 800 nm, resulting in absorption maxima at 584 nm. (FIG. 9C) The fluorescence profile of cSC^{mCherry} from 600 to 700 nm, upon excitation with 586 nm light.

FIGS. 10A-10E: Antigen-specific imaging using cSC^{mCherry} in complex with dimeric IgA that binds the Surface Layer Protein (SLP) of C. difficile. The cSFcA (CD5SLP-S^{mCherry}FcA) binds the antigen (SLP) through the sdAb-CD5SLP, which is fused to the IgA-Fc, and the bound cSC^{mCherry} confers the fluorescence, allowing the location of the antigen to be imaged. (FIG. 10A) Schematic of cSFcA (CD5SLP-S^{mCherry}FcA) with mCherry indicated as a star bound to SLP-coated bead (FIG. 10B). Brightfield image of cSFcA (CD5SLP-S^{mCherry}FcA) bound to SLP-coupled agarose resin beads. (FIG. 10C) Fluorescence image cSFcA (CD5SLP-S^{mCherry}FcA) bound to SLP-coupled agarose resin beads. (FIG. 10D) Control brightfield image of SLP-coupled agarose resin beads. (FIG. 10E) Control fluorescence image of SLP-coupled agarose resin beads.

FIGS. 11A-11C: (FIG. 11A) Schematic showing overall strategy for generating a library of cSC and cSIgA that target influenza viruses and for testing their potency in viral neutralization assays. (FIG. 11B) Neutralization of H1N1 influenza virus with cSC^{SD38}. Neutralization curves include cSC^{SD38} negative controls hSC and SIgA, and positive control antibody CR9114. (FIG. 11C) Neutralization of H3N2 influenza virus with cSC^{SD36}. Neutralization curves include cSC^{SD36}, negative controls hSC and SIgA, and positive control antibody CR9114. Results indicate that both cSC^{SD36} and CSC^{SD38} can neutralize virus.

### SEQUENCE LISTING

The Sequence Listing is submitted as an ST.26 Sequence Listing XML file, named 7950-106334-02, created on Sep. 9, 2022, having a size of 129,545 bytes, which is incorporated by reference herein. In the accompanying sequence listing: SEQ ID NO: 1 is the amino acid sequence of wild-type human SC containing a C-terminal hexahistidine affinity (His) tag.

SEQ ID NOs: 2-17 are amino acid sequences of recombinant human SC polypeptides containing a modified D2 domain.

SEQ ID NOs: 18-29 are amino acid sequences of recombinant murine SC polypeptides containing a modified D2 domain.

SEQ ID NOs: 30-82 are amino acid sequences of exemplary sdAbs and antibody Fab variable heavy (VH) or light (VL) chains that can replace the D2 domain of SC to confer antigen binding specificity.

SEQ ID NOs: 83-91 are amino acid sequences of exemplary fluorescent proteins that can replace the D2 domain.

SEQ ID NOs: 92-94 are amino acid sequences of exemplary immunoglobulin domains that can replace the D2 domain of SC.

SEQ ID NOs: 95-113 are amino acid sequences of exemplary proteins that can replace the D2 domain.

SEQ ID NO: 114 is the amino acid sequence of hSC-SD36-His.

SEQ ID NO: 115 is the amino acid sequence of hSC-SD38-His.

SEQ ID NOs: 116-118 are amino acid sequences of exemplary influenza virus hemagglutinin (HA)-specific sdAbs that can replace the D2 domain of SC to confer binding specificity.

### DETAILED DESCRIPTION

### I. Terms

Unless otherwise noted, technical terms are used according to conventional usage. Definitions of common terms in molecular biology may be found in Benjamin Lewin, Genes X, published by Jones & Bartlett Publishers, 2009; and Meyers et al. (eds.), The Encyclopedia of Cell Biology and Molecular Medicine, published by Wiley-VCH in 16 volumes, 2008; and other similar references.

As used herein, the singular forms “a,” “an,” and “the,” refer to both the singular as well as plural, unless the context clearly indicates otherwise. For example, the term “an antigen” includes single or plural antigens and can be considered equivalent to the phrase “at least one antigen.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting.

To facilitate review of the various implementations, the following explanations of terms are provided:

Administration: The introduction of a composition into a subject by a chosen route. Administration can be local or systemic. For example, if the chosen route is intravenous, the composition is administered by introducing the composition into a vein of the subject. Exemplary routes of administration include, but are not limited to, intranasal, inhalation, oral, injection (such as subcutaneous, intramuscular, intradermal, intraperitoneal, and intravenous), sublingual, rectal (such as by suppository), transdermal (for example, topical) and vaginal routes.

Angiotensin converting enzyme 2 (ACE2): A protein belonging to the angiotensin-converting enzyme family of peptidyl carboxydipeptidases and has considerable homology to human angiotensin 1 converting enzyme. ACE2 is a secreted protein that catalyzes the cleavage of angiotensin I into angiotensin 1-9, and angiotensin II into the vasodilator angiotensin 1-7. ACE2 is known to be expressed in various human organs, and its organ- and cell-specific expression suggests that it may play a role in the regulation of cardiovascular and renal function, as well as fertility. In addition, the encoded protein is a functional receptor for the spike glycoprotein of the human coronavirus HCoV-NL63 and the human severe acute respiratory syndrome coronaviruses, SARS-CoV and SARS-CoV-2. Nucleic acid and protein sequences of ACE2 are publicly available, such as under NCBI Gene ID 59272.

Antibody: A polypeptide ligand comprising at least one variable region that recognizes and binds (such as specifically recognizes and specifically binds) an epitope of an antigen. Mammalian immunoglobulin molecules are composed of a heavy (H) chain and a light (L) chain, each of which has a variable region, termed the variable heavy (VH) region and the variable light (V_{L}) region, respectively. Together, the V_{H} region and the V_{L} region are responsible for binding the antigen recognized by the antibody. There are five main heavy chain classes (or isotypes) of mammalian immunoglobulin, which determine the functional activity of an antibody molecule: IgM, IgD, IgG, IgA and IgE. Antibody isotypes not found in mammals include IgX, IgY, IgW and IgNAR. IgY is the primary antibody produced by birds and reptiles, and has some functionally similar to mammalian IgG and IgE. IgW and IgNAR antibodies are produced by cartilaginous fish, while IgX antibodies are found in amphibians.

Antibody variable regions contain “framework” regions and hypervariable regions, known as “complementarity determining regions” or “CDRs.” The CDRs are primarily responsible for binding to an epitope of an antigen. The framework regions of an antibody serve to position and align the CDRs in three-dimensional space. The amino acid sequence boundaries of a given CDR can be readily determined using any of a number of well-known numbering schemes, including those described by Kabat et al. (Sequences of Proteins of Immunological Interest, U.S. Department of Health and Human Services, 1991; the “Kabat” numbering scheme), Chothia et al. (see Chothia and Lesk, J Mol Biol 196:901-917, 1987; Chothia et al., Nature 342:877, 1989; and Al-Lazikani et al., (JMB 273,927-948, 1997; the “Chothia” numbering scheme), and the ImMunoGeneTics (IMGT) database (see, Lefranc, Nucleic Acids Res 29:207-9, 2001; the “IMGT” numbering scheme). The Kabat and IMGT databases are maintained online.

A “single-domain antibody (sdAb)” refers to an antibody having a single domain (a variable domain) that is capable of specifically binding an antigen, or an epitope of an antigen, in the absence of an additional antibody domain. Single-domain antibodies include, for example, V_{NAR} antibodies, camelid V_{H}H antibodies, V_{H} domain antibodies and V_{L} domain antibodies. V_{NAR} antibodies are produced by cartilaginous fish, such as nurse sharks, wobbegong sharks, spiny dogfish and bamboo sharks. Camelid V_{H}H antibodies are produced by several species including camel, llama, alpaca, dromedary, and guanaco, which produce heavy chain antibodies that are naturally devoid of light chains. In some implementations, the sdAb is fused to an Fc domain, such as a human or mouse Fc domain.

A “monoclonal antibody” is an antibody produced by a single clone of lymphocytes or by a cell into which the coding sequence of a single antibody has been transfected. Monoclonal antibodies are produced by methods known to those of skill in the art. Monoclonal antibodies include humanized monoclonal antibodies.

A “chimeric antibody” has framework residues from one species, such as human, and CDRs (which generally confer antigen binding) from another species, such as a V_{NAR} that specifically binds a viral antigen.

A “humanized” antibody is an immunoglobulin including a human framework region and one or more CDRs from a non-human (for example a shark, mouse, rabbit, rat, or synthetic) immunoglobulin. The non-human immunoglobulin providing the CDRs is termed a “donor,” and the human immunoglobulin providing the framework is termed an “acceptor.” In one implementation, all CDRs are from the donor immunoglobulin in a humanized immunoglobulin. Constant regions need not be present, but if they are, they must be substantially identical to human immunoglobulin constant regions, i.e., at least about 85-90%, such as about 95% or more identical. Hence, all parts of a humanized immunoglobulin, except possibly the CDRs, are substantially identical to corresponding parts of natural human immunoglobulin sequences. A humanized antibody binds to the same antigen as the donor antibody that provides the CDRs. Humanized or other monoclonal antibodies can have additional conservative amino acid substitutions which have substantially no effect on antigen binding or other immunoglobulin functions. Methods of humanizing shark V_{NAR} antibodies has been previously described (Kovalenko et al., J Biol Chem 288(24):17408-17419, 2013).

Antigen: A compound, composition, or substance that can stimulate the production of antibodies or a T-cell response in an animal, including compositions that are injected or absorbed into an animal. An antigen reacts with the products of specific humoral or cellular immunity, including those induced by heterologous immunogens. In some implementations herein, the antigen is a C. difficile antigen, such as low molecular weight (LMW) subunit of surface layer protein (SLP), flagellin (FliC), lipothechoic acid (LTA3), TcdA or TcdB; a Salmonella enterica antigen, such as FliC; a Salmonella Tm antigen, such as an O antigen, for example O5 antigen; a Staphylococcus aureus antigen, such as alpha toxin; a Campylobacter Jejuni antigen, such as FliD; a SARS-CoV-2 antigen, such as a SARS-CoV-2 spike protein; an HIV-1 antigen, such as an HIV-1 capsid protein or envelope protein; an influenza virus antigen, such as an influenza virus neuraminidase (NA) or hemagglutinin (HA) protein; or a norovirus antigen, such as a norovirus capsid antigen.

Chimeric: Composed of at least two parts having different origins.

Complementarity determining region (CDR): A region of hypervariable amino acid sequence that defines the binding affinity and specificity of an antibody. Single-domain antibodies, such as VH single-domain, VL single-domain, or camel VHH antibodies include three CDRs (CDR1, CDR2 and CDR3).

Endolysin: A hydrolytic enzyme produced by bacteriophages in order to cleave the host bacteria cell wall. Endolysins target one of the five bonds in bacterial peptidoglycan.

Fluorescent protein: A protein that emits light of a certain wavelength when exposed to a particular wavelength of light. Fluorescent proteins include, but are not limited to, green fluorescent proteins (such as GFP, EGFP, AcGFP1, Emerald, Superfolder GFP, Azami Green, mWasabi, TagGFP, TurboGFP and ZsGreen), blue fluorescent proteins (such as EBFP, EBFP2, Sapphire, T-Sapphire, Azurite and mTagBFP), cyan fluorescent proteins (such as ECFP, mECFP, Cerulean, CyPet, AmCyanl, Midori-Ishi Cyan, mTurquoise and mTFP1), yellow fluorescent proteins (EYFP, Topaz, Venus, mCitrine, YPet, TagYFP, PhiYFP, ZsYellow1 and mBanana), orange fluorescent proteins (Kusabira Orange, Kusabira Orange2, mOrange, mOrange2 and mTangerine), red fluorescent proteins (mRuby, mApple, mStrawberry, AsRed2, mRFP1, JRed, mCherry, HcRed1, mRaspberry, dKeima-Tandem, HcRed-Tandem, mPlum, AQ143, tdTomato and E2-Crimson), orange/red fluorescence proteins (dTomato, dTomato-Tandem, TagRFP, TagRFP-T, DsRed, DsRed2, DsRed-Express (T1) and DsRed-Monomer) and modified versions thereof. In some implementations herein, the fluorescent protein is mCherry, mRuby, mBanana, mTangerine, mStrawberry, mHoneydew, muGFP, mCardinal or miniSOG.

Heterologous: Originating from a separate genetic source or species. For example, a heterologous polypeptide or polynucleotide refers to a polypeptide or polynucleotide derived from a different source or species.

Human immunodeficiency virus (HIV): A retrovirus that causes immunosuppression in humans (HIV disease), and leads to a disease complex known as the acquired immunodeficiency syndrome (AIDS). “HIV disease” refers to a well-recognized constellation of signs and symptoms (including the development of opportunistic infections) in persons who are infected by HIV, as determined by antibody or western blot studies. Laboratory findings associated with this disease include a progressive decline in T cells. HIV includes HIV type 1 (HIV-1) and HIV type 2 (HIV-2).

Influenza virus (Influenza): Influenza type A and B viruses are RNA viruses that cause respiratory disease in humans. Influenza has two major surface antigens, hemagglutinin (HA) and neuraminidase (NA), which are involved in binding to host cells and facilitating viral-host cell fusion and downstream events, such as viral replication and dissemination, associated with disease. Influenza can be neutralized by antibodies that bind HA and NA; however rapid genome mutation allows influenza to evade many host antibody responses. Influenza causes seasonal epidemics of disease (known as flu season) in humans and related avian influenza causes seasonal epidemics of disease in birds. Avian influenza can be transmitted to humans and thus can be a source for zoonotic infections. Influenza strains infecting both humans and birds are considered to have pandemic potential.

Isolated: An “isolated” biological component has been substantially separated or purified away from other biological components, such as other biological components in which the component occurs, such as other chromosomal and extrachromosomal DNA, RNA, and proteins. Proteins, peptides, nucleic acids, and viruses that have been “isolated” include those purified by standard purification methods. Isolated does not require absolute purity, and can include protein, peptide, nucleic acid, or virus molecules that are at least 50% isolated, such as at least 75%, 80%, 90%, 95%, 98%, 99%, or even 99.9% isolated.

Modification: A change in the sequence of a nucleic acid or protein. For example, amino acid sequence modifications include, for example, substitutions, insertions and deletions, or combinations thereof. Insertions include amino and/or carboxyl terminal fusions as well as intrasequence insertions of single or multiple amino acid residues. Deletions are characterized by the removal of one or more amino acid residues from the protein sequence. In some implementations herein, the modification (such as a substitution, insertion or deletion) results in a change in a property of the polypeptide, such as the capacity to bind a target antigen or other molecule. Substitutional modifications are those in which at least one residue has been removed and a different residue inserted in its place. Amino acid substitutions are typically of single residues, but can occur at a number of different locations at once. Substitutions, deletions, insertions or any combination thereof may be combined to arrive at a final mutant sequence. These modifications can be prepared by modification of nucleotides in the DNA encoding the protein, thereby producing DNA encoding the modification. Techniques for making insertion, deletion and substitution mutations at predetermined sites in DNA having a known sequence are well-known. A “modified” protein or nucleic acid is one that has one or more modifications as outlined above.

Mucins: A family of high molecular weight, heavily glycosylated proteins produced by epithelial tissues in most animals.

Polypeptide: A polymer in which the monomers are amino acid residues joined together through amide bonds. When the amino acids are alpha-amino acids, either the L-optical isomer or the D-optical isomer can be used. The terms “polypeptide” and “protein” are used herein interchangeably and include standard amino acid sequences as well as modified sequences, such as glycoproteins. The term “polypeptide” is specifically intended to cover naturally occurring proteins, as well as proteins that are recombinantly or synthetically produced.

Pharmaceutically acceptable carriers: The pharmaceutically acceptable carriers of use are conventional. Remington: The Science and Practice of Pharmacy, 22^{nd} ed., London, UK: Pharmaceutical Press (2013), describes compositions and formulations suitable for pharmaceutical delivery of the recombinant polypeptides disclosed herein.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example, sodium acetate or sorbitan monolaurate. In particular implementations, suitable for administration to a subject the carrier may be sterile, and/or suspended or otherwise contained in a unit dosage form containing one or more measured doses of the composition suitable to treat or inhibit a bacterial or viral infection. It may also be accompanied by medications for its use for treatment purposes. The unit dosage form may be, for example, in a sealed vial that contains sterile contents or a syringe for injection into a subject, or lyophilized for subsequent solubilization and administration or in a solid or controlled release dosage. In some implementations, the pharmaceutical carrier includes chitosan (van der Lubben et al., Adv Drug Deliv Rev 52(2):139-144, 2001; Islam et al., Biomaterials 192:75-94, 2019), such as when using mucosal administration.

Preventing, treating or ameliorating a disease: “Preventing” a disease refers to inhibiting the full development of a disease. “Treating” refers to a therapeutic intervention that ameliorates a sign or symptom of a disease or pathological condition after it has begun to develop. “Ameliorating” refers to the reduction in the number or severity of signs or symptoms of a disease, such as a bacterial or viral infection.

Recombinant: A recombinant polypeptide or nucleic acid is one that has a sequence that is not naturally occurring or has a sequence that is made by an artificial combination of two otherwise separated segments of sequence.

SARS-CoV-2: A coronavirus of the genus betacoronavirus that first emerged in humans in 2019. This virus is also known as Wuhan coronavirus, 2019-nCoV, or 2019 novel coronavirus. Symptoms of SARS-CoV-2 infection include fever, chills, dry cough, shortness of breath, fatigue, muscle/body aches, headache, new loss of taste or smell, sore throat, nausea or vomiting, and diarrhea. Patients with severe disease can develop pneumonia, multi-organ failure, and death. The time from exposure to onset of symptoms is approximately 2 to 14 days. The SARS-CoV-2 virion includes a viral envelope with large spike glycoproteins. The SARS-CoV-2 genome, like most coronaviruses, has a common genome organization with the replicase gene included in the 5′-two thirds of the genome, and structural genes included in the 3′-third of the genome. The SARS-CoV-2 genome encodes the canonical set of structural protein genes in the order 5′-spike (S)-envelope (E)-membrane (M) and nucleocapsid (N)-3′.

SARS Spike (S) protein: A class I fusion glycoprotein initially synthesized as a precursor protein of approximately 1256 amino acids for SARS-CoV, and 1273 amino acids for SARS-CoV-2. Individual precursor S polypeptides form a homotrimer and undergo glycosylation within the Golgi apparatus as well as processing to remove the signal peptide, and cleavage by a cellular protease between approximately position 679/680 for SARS-CoV, and 685/686 for SARS-CoV-2, to generate separate S1 and S2 polypeptide chains, which remain associated as S1/S2 protomers within the homotrimer, thereby forming a trimer of heterodimers. The S1 subunit is distal to the virus membrane and contains the receptor-binding domain (RBD) that is believed to mediate virus attachment to its host receptor. The S2 subunit is believed to contain the fusion protein machinery, such as the fusion peptide. S2 also includes two heptad-repeat sequences (HR1 and HR2) and a central helix typical of fusion glycoproteins, a transmembrane domain, and a cytosolic tail domain.

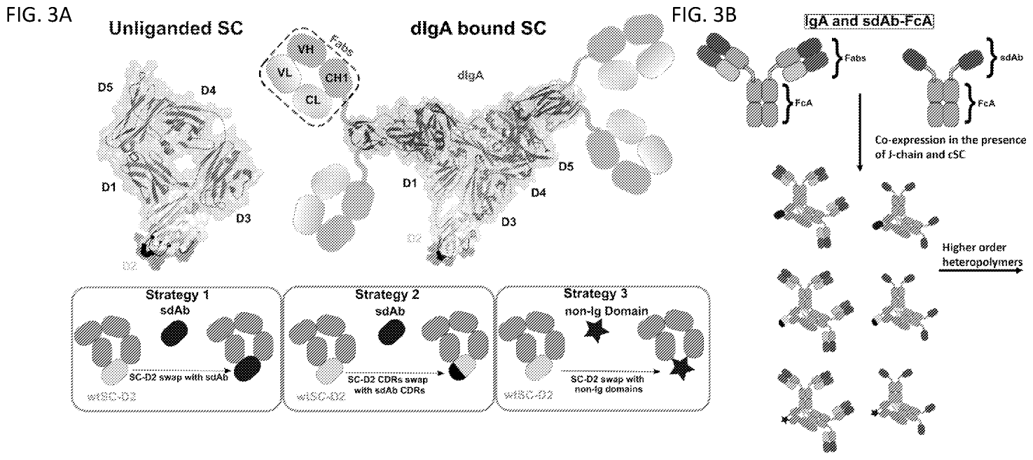

Secretory component (SC): The ectodomain of the polyimmunoglobulin receptor (pIgR). SC is also part of secretory immunoglobulin A (sIgA) and M (sIgM), which are respectively comprised of at least two monomeric IgA molecules and at least five IgM molecules (linked by the J chain) and SC. Polymeric forms of IgA and IgM bind the pIgR on the basolateral surface of epithelial cells and enter cells by transcytosis. The pIgR/polymeric IgA/IgM complex passes through cellular compartments and is then secreted on the luminal surface of epithelial cells, which is followed by proteolysis of the pIgR, resulting in sIgA or sIgM. SC contains five domains—D1, D2, D3, D4 and D5 (see FIG. 3). In some implementations of the present disclosure, the D2 domain is from human SC. In some examples, wild-type human SC is at least 90%, at least 95%, at least 96%, at least 97%, at least 98%, at least 99% or 100% identical to SEQ ID NO: 1. Similarly, in some examples, wild-type D2 is at least 90%, at least 95%, at least 96%, at least 97%, at least 98%, at least 99% or 100% identical to residues 136-236 of SEQ ID NO: 1. In other implementations, the D2 domain is from another mammalian species, such as mouse.

Specific binding pair: Two molecules that interact by means of specific, non-covalent interactions that depend on the three-dimensional structures of the molecules involved. Exemplary specific binding pairs include antigen/antibody, hapten/antibody, ligand/receptor, substrate/enzyme, inhibitor/enzyme, carbohydrate/lectin, biotin/streptavidin, and virus/cellular receptor. Particular examples of specific binding pairs disclosed herein include, but are not limited to, Spr1345 and mucin; an angiotensin converting enzyme 2 (ACE2) polypeptide and the SARS-CoV-2 spike protein receptor binding domain; CD4 and HIV-1 gp120; streptavidin and biotin; sialic acid-binding Ig-like lectin 12 (Siglec-12) and sialic acid; sialic acid-binding Ig-like lectin 15 (Siglec-15) and sialic acid; azurin and copper; retinol binding protein-II and retinol; galectin-4 and lactose; galectin-8 and lactose; and trbp111 and tRNA.

Subject: Living multi-cellular vertebrate organisms, a category that includes both human and veterinary subjects, including human and non-human mammals such as birds, pigs, mice, rats, rabbits, sheep, horses, cows, dogs, cats and non-human primates). In some implementations, the subject is a human. In some examples, the subject is a human subject with a bacterial or viral infection.

Therapeutically effective amount: A quantity of a specific substance, such as a disclosed recombinant polypeptide, sufficient to achieve a desired effect in a subject being treated. A “therapeutically effective amount” can be the amount necessary to inhibit viral or bacterial replication or to treat a subject with an existing viral or bacterial infection. Similarly, a “prophylactically effective amount” refers to administration of an agent or composition in an amount that inhibits or prevents establishment of an infection, such as a viral or bacterial infection. In some implementations herein, the therapeutically or prophylactically effective amount is the amount of a recombinant polypeptide sufficient to prevent, treat (including prophylaxis), reduce and/or ameliorate the symptoms and/or underlying causes of a disease or disorder, for example to prevent, inhibit, and/or treat a viral or bacterial infection. In some implementations, a therapeutically effective amount is sufficient to reduce or eliminate a symptom of a disease, such as a bacterial or viral infection. For instance, this can be the amount necessary to inhibit or prevent viral/bacterial replication or to measurably alter outward symptoms of the viral/bacterial infection. In general, this amount will be sufficient to measurably inhibit virus/bacterial replication or infectivity.

In one example, a desired response is to inhibit or reduce or prevent a viral or bacterial infection. The infection does not need to be completely eliminated or reduced or prevented for the method to be effective. For example, administration of a therapeutically effective amount of the agent can decrease the infection (for example, as measured by infection of cells, or by number or percentage of subjects infected by the virus/bacteria) by a desired amount, for example by at least 10%, at least 20%, at least 50%, at least 60%, at least 70%, at least 80%, at least 90%, at least 95%, at least 98%, or even at least 100% (elimination or prevention of detectable infection, as compared to a suitable control).

A therapeutically effective amount of an agent can be administered in a single dose, or in several doses, for example daily, during a course of treatment. However, the therapeutically effective amount can depend on the subject being treated, the severity and type of the condition being treated, and the manner of administration. A unit dosage form of the agent can be packaged in a therapeutic amount, or in multiples of the therapeutic amount, for example, in a vial (e.g., with a pierceable lid) or syringe having sterile components.

### II. Secretory Component and Secretory IgA

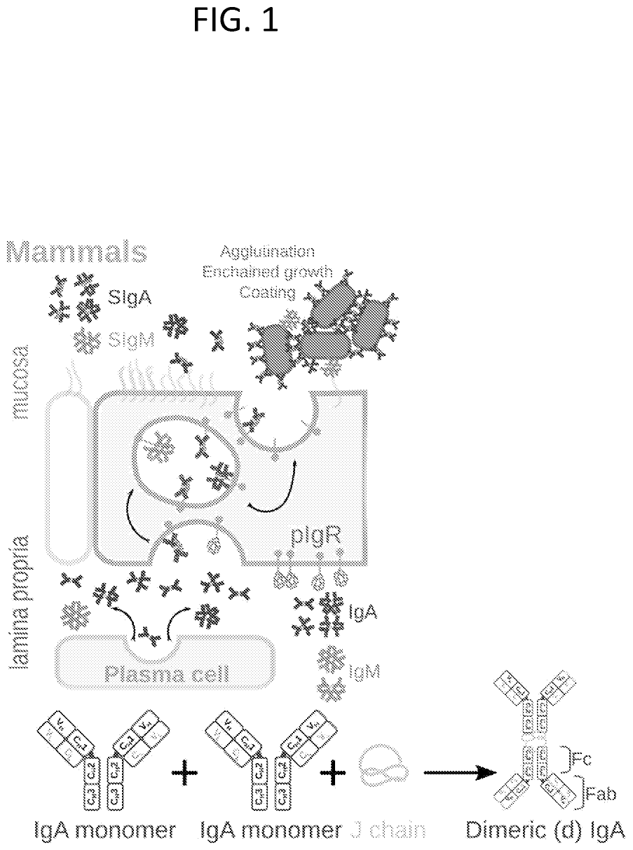

The present disclosure investigates the therapeutic potential of SC and associated polymeric immunoglobulins (pIg), which populate the mucosa and mediate host interactions with toxins, pathogens and commensal organisms (Flajnik, Nat Immunol 11(9):777-779, 2010; Kaetzel, ISRN Immunology 2014:20, 2014). The pIgs include several Ig heavy chain classes, such as IgA and IgM in mammals, birds and reptiles, and IgM and IgT (also called IgZ) in teleost fish (Flajnik, Nat Immunol, 2010. 11(9):777-9; Sunyer, Nat Immunol, 2013. 14(4):320-6). These pIgs typically contain between two and five Ig monomers, each with two copies of the heavy chain and two copies of the light chain that together form two antigen binding fragments (Fabs) and one fragment crystallization (Fc). The majority of pIgs are assembled in plasma cells with one copy of a protein called the joining-chain (JC); however, the potential to associate with the JC and/or to assemble into polymers of different size varies with species, Ig heavy chain class, isoform and allotype (FIG. 1) (Flajnik, Nat Immunol, 2010. 11(9):777-9; Woof and Russell, Mucosal Immunol, 2011. 4(6):590-7).

Following assembly, pIgs are transported through epithelial cells by the polymeric Ig receptor (pIgR) and released into the mucosa. There, the pIgR ectodomain, called secretory component (SC), remains bound to the Fc and the antibody is referred to as a secretory Ig (SIg). In the mucosa, SIg are associated with unique effector functions compared to monomeric, circulatory antibodies, which depend on antigen interactions with Fabs and also have the capacity to bind host and microbial receptors. SIgA is the predominant mucosal antibody (others being sIgM and IgG) in mammals and mediates physical mechanisms such as antigen coating, cross-linking, agglutination and high avidity interactions; outcomes are diverse and typically not associated with inflammation (Woof and Russell, Mucosal Immunol, 2011. 4(6):590-7; Pabst and Slack, Mucosal Immunol, 2020. 13(1):12-21) (FIG. 1). SIgA can enchain dividing human pathogen Salmonella enterica, and protect against opportunistic pathogens such as Clostridium difficile, yet also promote growth of commensal microbes and when ingested through colostrum and breastmilk, provide passive immunity to newborns and impact microbiome composition for life (Moor et al., Nature, 2017. 544(7651):498-502; Donaldson et al., Science, 2018. 360(6390):795-800; Rogier et al., Proc Natl Acad Sci USA, 2014. 111(8):3074-9). SIgA has considerable therapeutic potential, particularly for countering human pathogens such as C. difficile infection (CDI), which is the most common hospital acquired infection in the United States, with up to a 17% death rate, and cumulatively increasing recurrences of 20%-35% (Yang et al., J Infect Dis, 2014. 210(6):964-72). IgG-based antibody treatments (e.g., Bezlotoxumab) for CDI have shown promise; yet host SIgA can provide resistance to CDI and therefore therapeutic SIgA (and SIgM) represents a largely unexplored avenue for CDI treatment with advantages over IgGs (Hussack and Tanha, Clin Exp Gastroenterol, 2016. 9:209-24; Bridgman et al., Microbes Infect, 2016. 18(9):543-9; Stubbe et al., J Immunol, 2000. 164(4):1952-60).

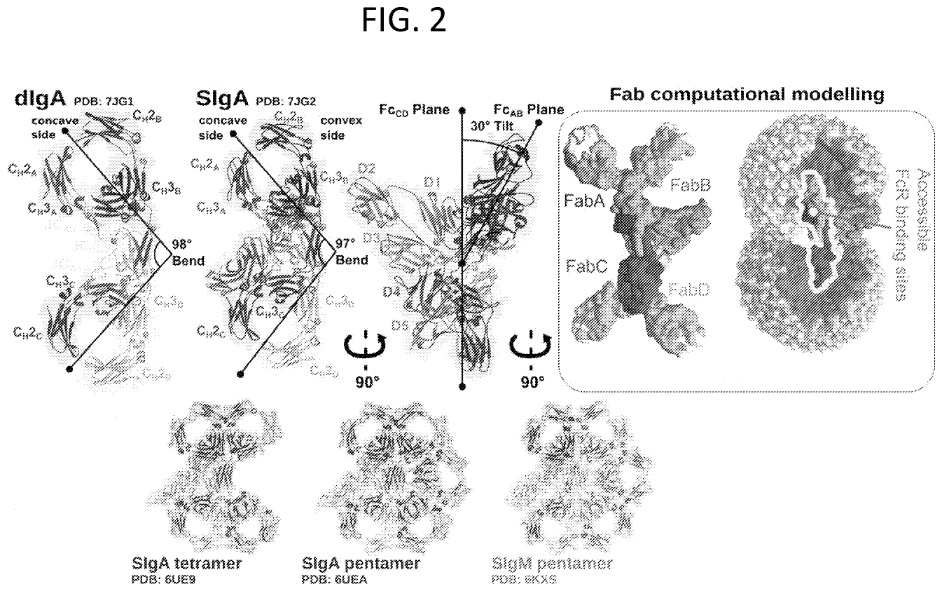

Despite the significance, the structural basis for SIg function in the mucosa remained poorly understood through decades of immunological research. However, cryo-electron microscopy (cryoEM) structures of mouse dimeric IgA (dIgA) and SIgA (FIG. 2) were reported in Kumar Bharathkar et al. (Elife, 2020. 9:e56098). Structures of human SIgA and SIgM have also been reported (Kumar Bharathkar et al., Elife, 2020. 9:e56098; Kumar et al., Science, 2020. 367(6481):1008-1014; Li et al., Science, 2020. 367(6481):1014-1017; Wang et al., Cell Res, 2020. 30(7):602-609) (FIG. 2).

In the mouse and human SIgA structures, two IgA monomers are bound by the JC and the SC to form an asymmetric complex with concave and convex sides. The five Ig-like domains (D1-D5) of SC are bound to one face, asymmetrically contacting both IgAs and JC and occupying a solvent accessible location on one side of the molecule (FIG. 2). Computation modeling suggested that possible positions SIgA Fabs adopt are directed toward the concave side of the antibody, preserving accessibility to Fc receptor (FcR) binding sites located on the convex side and leaving parts of SC, including D2, exposed to solvent (FIG. 2) (Kumar Bharathkar et al., Elife, 2020. 9:e56098). These results indicated that the asymmetric conformation of a SIg has the potential to influence functions such as antigen binding.

Concurrently reported structures of tetrameric and pentameric forms of human SIgA and pentameric forms of human SIgM revealed that heavy chain C-terminal β-sheets (called tailpieces) “stack” as antibody polymer size increases; however, JC and SC adopted similar conformations and contacts with neighboring components in all structures, with the exception of SC D2 which adopts flexible positions (FIG. 2) (Kumar Bharathkar et al., Elife, 2020. 9:e56098; Kumar et al., Science, 2020. 367(6481):1008-1014; Li et al., Science, 2020. 367(6481):1014-1017).

SC has been associated with protecting SIg from proteolysis, interacting with host and microbial lectins and binding Streptococcus pneumoniae surface protein CbpA; however, these and other putative functions are only partly understood (Wang et al., Cell Res, 2020. 30(7):602-609; Kaetzel, Immunol Rev, 2005. 206:83-99). In mammals, SC has five domains, D1-D5, each having an Ig-like fold with loops structurally similar to antibody CDRs. When unliganded, these domains adopt a compact conformation (Stadtmueller et al., Elife, 2016. 5:e10640). In the murine SIgA structure (and human SIgA and SIgM structures) SC is extended and exhibits significant accessible surface area (in excess of 25,000 Å²) leaving it well-positioned to interact with host or microbial factors. D2 is particularly accessible, being located distal from SIgA's center where it forms limited contacts with other complex components (FIG. 2) (Kumar Bharathkar et al., Elife, 2020. 9:e56098). D2-specific ligands have not been reported. However, the present disclosure describes functionalization of D2 to evaluate the ligand binding capacity and therapeutic potential of SC.

To evaluate the functional and therapeutic potential of SC and its complexes with IgA and IgM, interactions with C. difficile and influenza virus were investigated. The mechanisms of normal SIgA-based protection against CDI were not well understood and its use as a therapeutic has not previously been well explored (Hussack and Tanha, Clin Exp Gastroenterol, 2016. 9:209-24; Bridgman et al., Microbes Infect, 2016. 18(9):543-9; Stubbe et al., J Immunol, 2000. 164(4):1952-60; Dallas and Rolfe, J Med Microbiol, 1998. 47(10):879-88). The present disclosure describes an engineered chimeric SC that can bind C. difficile toxin TcdA though a modified D2 domain. Further disclosed are chimeric SC that bind influenza virus hemagglutinin (HA) by replacement of the D2 domain with a single-domain antibody that binds HA (SD36 or SD38).

### III. Overview of Several Implementations

Disclosed herein are recombinant polypeptides that include a chimeric secretory component (cSC) protein in which the D2 domain of secretory component is modified to confer one or more non-native properties to the polypeptide. Structural studies of SIgA showed that SC is solvent accessible, making it a possible target for engineering unique binding specificity into SC, SIgA and SIgM. Thus, as described herein, the D2 domain can be modified, for example, to confer specific binding to a target molecule, such as by replacing the D2 domain with a single domain antibody or by modifying the D2 domain by replacement of complementarity determining region (CDR)-like loops with CDR sequences from a single domain antibody. Binding specificity of the D2 domain can also be achieved by modification (such as substitution) of the D2 domain with one member of a specific binding pair, or with an endolysin (to target bacterial peptidoglycan). In some examples, the specific binding pair includes an enzyme. The D2 domain can also be modified to enable fluorometric or colorimetric detection of the recombinant polypeptide. Methods of using the recombinant polypeptides, such as for treating or inhibiting a microbial infection, are also described. In some examples of these methods, the D2 domain of the recombinant polypeptide is modified to confer specific binding to a microbial antigen, sialic acid or lactose.

Provided herein are recombinant polypeptides that include a chimeric secretory component (cSC) protein. In the disclosed recombinant polypeptides, the D2 domain of the cSC includes at least one modification that confers specific binding to a target molecule or enables fluorometric or colorimetric detection of the recombinant polypeptide.

In some implementations of the recombinant polypeptide, the at least one modification of the D2 domain includes substitution of CDR-like loops of the D2 domain with CDRs of a single-domain antibody, a variable heavy (VH) domain or a variable light (VL) domain; substitution of the D2 domain with a single-domain antibody, a VH domain or a VL domain; substitution of the D2 domain with a first member of a specific binding pair; substitution of the D2 domain with an endolysin; substitution of the D2 domain with a fluorescent protein; or substitution of the D2 domain with Azurin, which detects Cu(I) by turning blue and acts as a colorimetric detection moiety.

In some implementations of the disclosed recombinant polypeptides, the at least one modification of the D2 domain includes substitution of CDR-like loops of the D2 domain with CDRs of a single-domain antibody, a VH domain or a VL domain; and the target molecule is an antigen. In particular examples, the CDR sequences of the single-domain antibody, the VH domain or the VL domain are the CDR sequences of any one of SEQ ID NOs: 30-82 and 116-118. One of skill in the art can readily determine the locations of each CDR in an amino acid sequence using any known convention, such as IMGT, Kabat or Chothia. In specific non-limiting examples, the amino acid sequence of the recombinant polypeptide is at least 80%, at least 85%, at least 90%, at least 95%, at least 96%, at least 97%, at least 98%, or at least 99% identical to SEQ ID NO: 6 or SEQ ID NO: 7, or comprises or consists of SEQ ID NO: 6 or SEQ ID NO: 7.

In other implementations of the disclosed recombinant polypeptides, the at least one modification of the D2 domain includes substitution of the D2 domain with a single-domain antibody, a VH domain or a VL domain; and the target molecule is an antigen. In particular examples, the amino acid sequence of the single-domain antibody, VH domain or VL domain is at least 80%, at least 85%, at least 90%, at least 95%, at least 96%, at least 97%, at least 98%, or at least 99% identical to any one of SEQ ID NOs: 30-82 and 116-188, or comprises or consists of any one of SEQ ID NOs: 30-82 and 116-118. In specific non-limiting examples, the amino acid sequence of the recombinant polypeptide is at least 80%, at least 85%, at least 90%, at least 95%, at least 96%, at least 97%, at least 98%, or at least 99% identical to any one of SEQ ID NOs: 1-5, 8-29, 114 and 115, or comprises or consists of any one of SEQ ID NOs: 1-5, 8-29, 114 and 115.

In some examples, the antigen is a bacterial antigen. In specific examples, the bacterial antigen is an antigen of Clostridium difficile, Salmonella enterica, Salmonella Tm, Streptococcus pneumoniae, Staphylococcus aureus Listeria monocytogenes or Campylobacter Jejuni. In particular non-limiting examples, the C. difficile antigen includes the low molecular weight (LMW) subunit of surface layer protein (SLP), flagellin (FliC), lipothechoic acid (LTA3), TcdA or TcdB; the Salmonella enterica antigen includes FliC; the Salmonella Tm antigen includes an O antigen, such as the O5 antigen; the Staphylococcus aureus antigen includes alpha toxin; or the Campylobacter Jejuni antigen includes FliD.

In other examples, the antigen is a viral antigen. In specific examples, the viral antigen is an antigen of human immunodeficiency virus (HIV)-1, severe acute respiratory syndrome coronavirus 2 (SARS-CoV-2), influenza virus or norovirus. In particular non-limiting examples, the SARS-CoV-2 antigen includes a SARS-CoV-2 spike protein or nucleocapsid protein; the HIV-1 antigen includes an HIV-1 capsid protein, gp120, gp41 or p24, or envelope protein; the influenza virus antigen is HA or NA; or the norovirus antigen includes a norovirus capsid antigen, VP1 or VP2.

In other implementations of the recombinant polypeptide, the at least one modification of the D2 domain includes substitution of the D2 domain with a first member of a specific binding pair; and the target molecule is a second member of the specific binding pair. In some examples, the first and second members of the specific binding pair respectively include: Spr1345 and mucin; an angiotensin converting enzyme 2 (ACE2) polypeptide and a SARS-CoV-2 spike protein receptor binding domain; CD4 and HIV-1 gp120; streptavidin and biotin; sialic acid-binding Ig-like lectin 12 (Siglec-12) and sialic acid; sialic acid-binding Ig-like lectin 15 (Siglec-15) and sialic acid; azurin and copper; retinol binding protein-II and retinol; galectin-4 and lactose; galectin-8 and lactose; trbp111 and tRNA; bile acid binding protein and bile acid; beta lactoglobulin and a hydrophobic compound; F17b-G lectin domain and lectin; MucBP domain of LBA1460 and mucin; MucBP domain of PEPE and mucin; nectin-3 ectodomain and TcdB; MAdCAM-1 and integrin α4β7; defensin-5 and bacteria (Gram-positive or Gram-negative); defensin-6 and bacteria (Gram-positive or Gram-negative); FedF adhesion protein and lectin; Lactobacilli mub-RV and mucin (see, for example, Tables 3 and 4). In particular non-limiting examples, the amino acid sequence of the first member of the specific binding pair is at least 80%, at least 85%, at least 90%, at least 95%, at least 96%, at least 97%, at least 98%, or at least 99% identical to any one of SEQ ID NOs: 92-113, or comprises or consists of any one of SEQ ID NOs: 92-113. In some examples, the first member and/or second member of the specific binding pair is a portion/fragment of the molecule that retains the ability to bind to the other member.

In other implementations of the recombinant polypeptide, the at least one modification of the D2 domain includes substitution of the D2 domain with an endolysin; and the target molecule is a bacterial peptidoglycan. In some examples, the bacterial peptidoglycan is from Clostridium difficile, Streptococcus pyogenes, Streptococcus uberis, Streptococcus equi, Streptococcus gordinii, Streptococcus intermedius, Streptococcus parasanguis, Streptococcus pneumoniae, Enterococcus faecalis, Enterococcus faecium, Staphylococcus aureus, Bacillus anthracis, Bacillus cereus, Bacillus thuringiensis or Bacillus megaterium. In some examples, the endolysin includes CD27L, PlyC, PlyGBS, Cpl-1, PlyV12, ClyS, PlyB, PlyG or PlyPH (see Table 5).

In other implementations of the recombinant polypeptide, the at least one modification of the D2 domain includes substitution of the D2 domain with a fluorescent protein. In some examples, the fluorescent protein is mCherry, mRuby, mBanana, mTangerine, mStrawberry, mHoneydew, muGFP, mCardinal or miniSOG. In particular examples, the amino acid sequence of the fluorescent protein is at least 80%, at least 85%, at least 90%, at least 95%, at least 96%, at least 97%, at least 98%, or at least 99% identical to any one of SEQ ID NOs: 83-91, or comprises or consists of any one of SEQ ID NOs: 83-91.

In some implementations, the recombinant polypeptide further includes a polymeric IgA (such as dimeric, trimeric, tetrameric or pentameric IgA) or polymeric IgM (e.g., see FIG. 3). In some examples, the polymeric IgA or polymeric IgM specifically binds a mucosal protein or an antigen. In specific examples, the mucosal protein is a mucin and the mucosal antigen is a C. difficile protein or toxin. SIgA or SIgM comprising a chimeric SC provides the potential for crosslinking and/or high avidity interactions associated with normal SIgA and SIgM functions while adding additional binding capabilities, thereby making it a chimeric, bispecific antibody. Recombinant polypeptides that include SIgA with a modified D2 domain (as described herein) may be particularly effective for treating C. difficile infection (CDT) because pathogenesis is associated with both secreted toxins and by persistent C. difficile growth. In non-limiting examples, SIgA with cSC binds one antigen, such as a C. difficile toxin, via the cSC, and binds another antigen, such as the C. difficile surface layer protein (SLP), with the Fabs. In other non-limiting examples, a cSC binds influenza virus HA while the Fabs bind another influenza virus protein (such as NA). Chimeric, bispecific SIgA (or SIgM) can be delivered as an oral therapeutic similar to colostrum and milk SIgAs that have been shown to provide resistance to CDI (Dallas and Rolfe, J Med Microbiol, 1998. 47(10):879-88; Schmautz et al., PLoS One, 2018. 13(4):e0195275). Furthermore, cSC and cSIgA can be engineered to bind host mucins which populate the mucosa and are commonly bound to pathogenic agents, including C. difficile spore coat protein CotE (Hong et al., J Infect Dis, 2017. 216(11):1452-1459). Targeting cSC or chimeric SIgA to mucins can be used to both direct its location and inhibit pathogen binding to a host factor. In these examples, the D2 domain can be modified by substitutions of a sdAb (or CDR sequences thereof) that bind mucin, or D2 can be modified by substitution with a microbial mucin-binding domain, a subset of which adopt a compact structure that could replace D2 (Di et al., J Struct Biol, 2011. 174(1):252-7).

Further provided herein are nucleic acid molecules encoding a recombinant polypeptide disclosed herein. In some implementations, the nucleic acid molecule encoding the recombinant polypeptide is operably linked to a promoter, such as a heterologous promoter. Also provided are vectors that include a recombinant polypeptide-encoding nucleic acid molecule. Host cells that include a nucleic acid molecule or vector are further provided.

Also provided herein are methods of treating or inhibiting a Clostridium difficile, Salmonella enterica, Salmonella Tm, Streptococcus pneumoniae, Staphylococcus aureus or Campylobacter Jejuni infection in a subject. In some implementations, the method includes administering to the subject a therapeutically or prophylactically effective amount of a recombinant polypeptide disclosed herein. In some examples of these methods, the D2 domain of the recombinant polypeptide is modified to confer specific binding to a Clostridium difficile, Salmonella enterica, Salmonella Tm, Streptococcus pneumoniae, Staphylococcus aureus or Campylobacter Jejuni antigen, such as by substitution of the D2 domain with a single-domain antibody that specifically binds the antigen, or substitution of the CDR-like loops of the D2 domain with the CDRs of a single-domain antibody that specifically binds the antigen. In other examples, the D2 domain can be modified by substitution with an endolysin or with (for example) a protein that binds a mucin, lectin, integrin, or sialic acid.

Further provided are methods of treating or inhibiting an HIV-1, SARS-CoV-2, influenza virus or norovirus infection in a subject. In some implementations, the method includes administering to the subject a therapeutically or prophylactically effective amount of a recombinant polypeptide disclosed herein. In some examples of these methods, the D2 domain of the recombinant polypeptide is modified to confer specific binding to a HIV-1, SARS-CoV-2, influenza virus or norovirus antigen, such as by substitution of the D2 domain with a single-domain antibody that specifically binds the antigen, or substitution of the CDR-like loops of the D2 domain with the CDRs of a single-domain antibody that specifically binds the antigen. In other examples, the D2 domain can be modified by substitution with a polypeptide that binds the virus or viral antigen, such as a CD4 or ACE2 polypeptide.

In some implementations of the methods disclosed herein, the recombinant polypeptide is administered orally, intranasally or as a suppository. In other implementations, the recombinant polypeptide is administered intravenously, intraperitoneally or by inhalation.

### IV. Recombinant Secretory Component Amino Acid Sequences

The recombinant polypeptides disclosed herein contain a secretory component (such as human or mouse secretory component) in which the D2 domain contains at least one modification that confers one or more non-native properties to the polypeptide, such as specific binding to a microbial antigen. This section provides exemplary antibody, protein and polypeptide sequences (or relevant portions thereof, such as CDR sequences) that can substitute for the D2 domain of SC to generate a recombinant polypeptide.

A. Modifications to confer antigen binding by replacement of the D2 domain with single-domain antibodies or CDR sequences thereof

Provided below are exemplary amino acid sequences of a series of recombinant polypeptides that include human or mouse SC having a modified D2 domain that confers antigen binding specificity. In each amino acid sequence listed below, the N-terminal signal sequence and the C-terminal His tag are indicated by italics and the D2 domain (either a WT, modified or substituted D2 domain) is underlined. The bold residues in SEQ ID NO: 1 represent the CDR-like loops of the WT D2 domain. The bold residues in SEQ ID NOs: 6 and 7 represent the CDR sequences substituted into the D2 domain. The “GS” and “SG” residues at the N-terminus and C-terminus (respectively) of the D2 domains of SEQ ID NOs: 2-5 and 8-12 are linkers. Table 1 provides additional information about each of the modified D2 domains, including the species, strain and antigen specificity conferred by the modification(s). Table 2 provides exemplary antibody sequences (such as sdAb, VH or VL sequences) that can be substituted for the D2 domain. Alternatively, the CDR sequences of any of the antibodies listed in Table 2 can replace the CDR-like loops of the D2 domain.

WT human secretory component (hSC)-His
(SEQ ID NO: 1)
MLLFVLTCLLAVFPAISTKSPIFGPEEVNSVEGNSVSITCYYPPT
SVNRHTRKYWCRQGARGGCITLISSEGYVSSKYAGRANLTNFPEN
GTFVVNIAQLSQDDSGRYKCGLGINSRGLSFDVSLEVSQGPGLLN
DTKVYTVDLGRTVTINCPFKTENAQKRKSLYKQIGLYPVLVIDSS
GYVNPNYTGRIRLDIQGTGQLLFSVVINQLRLSDAGQYLCQAGDD
SNSNKKNADLQVLKPEPELVYEDLRGSVTFHCALGPEVANVAKFL
CRQSSGENCDVVVNTLGKRAPAFEGRILLNPQDKDGSFSVVITGL
RKEDAGRYLCGAHSDGQLQEGSPIQAWQLFVNEESTIPRSPTVVK
GVAGSSVAVLCPYNRKESKSIKYWCLWEGAQNGRCPLLVDSEGWV
KAQYEGRLSLLEEPGNGTFTVILNQLTSRDAGFYWCLTNGDTLWR
TTVEIKIIEGEPNLKVPGNVTAVLGETLKVPCHFPCKFSSYEKYW
CKWNNTGCQALPSQDEGPSKAFVNCDENSRLVSLTLNLVTRADEG
WYWCGVKQGHFYGETAAVYVAVEERGSHHHHHH
hSC-A20.1-His
(SEQ ID NO: 2)
MLLFVLTCLLAVFPAISTKSPIFGPEQVNSVEGNSVSITCYYPPT
SVNRHTRKYWCRQGARGGCITLISSEGYVSSKYAGRANLTNFPEN
GTFVVNIAQLSQDDSGRYKCGLGINSRGLSFDVSLEVSQGPGLLN
GSQVQLVESGGGLAQAGGSLRLSCAASGRTFSMDPMAWFRQPPGK
EREFVAAGSSTGRTTYYADSVKGRFTISRDNAKNTVYLQMNSLKP
EDTAVYYCAAAPYGANWYRDEYDYWGQGTQVTVSSSGKPEPELVY
EDLRGSVTFHCALGPEVANVAKFLCRQSSGENCDVVVNTLGKRAP
AFEGRILLNPQDKDGSFSVVITGLRKEDAGRYLCGAHSDGQLQEG
SPIQAWQLFVNEESTIPRSPTVVKGVAGSSVAVLCPYNRKESKSI
KYWCLWEGAQNGRCPLLVDSEGWVKAQYEGRLSLLEEPGNGTFTV
ILNQLTSRDAGFYWCLTNGDTLWRTTVEIKIIEGEPNLKVPGNVT
AVLGETLKVPCHFPCKFSSYEKYWCKWNNTGCQALPSQDEGPSKA
FVNCDENSRLVSLTLNLVTRADEGWYWCGVKQGHFYGETAAVYVA
VEERGSHHHHHH
hSC-A5.1-His
(SEQ ID NO: 3)
MLLFVLTCLLAVFPAISTKSPIFGPEEVNSVEGNSVSITCYYPPT
SVNRHTRKYWCRQGARGGCITLISSEGYVSSKYAGRANLTNFPEN
GTFVVNIAQLSQDDSGRYKCGLGINSRGLSFDVSLEVSQGPGLLN
GSQVKLEESGGGLVQAGGSLRLSCAASGRTFSMYRMGWFRQAPGK
EREFVGVITRNGSSTYYADSVKGRFTISRDNAKNTVYLQMNSLKP
EDTALYYCAATSGSSYLDAAHVYDYWGQGTQVTVSSSGKPEPELV
YEDLRGSVTFHCALGPEVANVAKFLCRQSSGENCDVVVNTLGKRA
PAFEGRILLNPQDKDGSFSVVITGLRKEDAGRYLCGAHSDGQLQE
GSPIQAWQLFVNEESTIPRSPTVVKGVAGSSVAVLCPYNRKESKS
IKYWCLWEGAQNGRCPLLVDSEGWVKAQYEGRLSLLEEPGNGTFT
VILNQLTSRDAGFYWCLTNGDTLWRTTVEIKIIEGEPNLKVPGNV
TAVLGETLKVPCHFPCKFSSYEKYWCKWNNTGCQALPSQDEGPSK
AFVNCDENSRLVSLTLNLVTRADEGWYWCGVKQGHFYGETAAVYV
AVEERGSHHHHHH
hSC-CD5SLP-His
(SEQ ID NO: 4)
MLLFVLTCLLAVFPAISTKSPIFGPEEVNSVEGNSVSITCYYPPT
SVNRHTRKYWCRQGARGGCITLISSEGYVSSKYAGRANLTNFPEN
GTFVVNIAQLSQDDSGRYKCGLGINSRGLSFDVSLEVSQGPGLLN
GSQVKLEESGGGLVQAGGSLRLSCAASRLTESTYHMGWFRQAPGK
EREFVAALSWSGGTTYYADSVKGRFGISRDNAKNTVYLQMNSLKP
EDTAVYYCASGGVLATMNSDEYDYWGQGTQVTVSSSGKPEPELVY
EDLRGSVTFHCALGPEVANVAKFLCRQSSGENCDVVVNTLGKRAP
AFEGRILLNPQDKDGSFSVVITGLRKEDAGRYLCGAHSDGQLQEG
SPIQAWQLFVNEESTIPRSPTVVKGVAGSSVAVLCPYNRKESKSI
KYWCLWEGAQNGRCPLLVDSEGWVKAQYEGRLSLLEEPGNGTFTV
ILNQLTSRDAGFYWCLTNGDTLWRTTVEIKIIEGEPNLKVPGNVT
AVLGETLKVPCHFPCKFSSYEKYWCKWNNTGCQALPSQDEGPSKA
FVNCDENSRLVSLTLNLVTRADEGWYWCGVKQGHFYGETAAVYVA
VEERGSHHHHHH
hSC-CDB1-His
(SEQ ID NO: 5)
MLLFVLTCLLAVFPAISTKSPIFGPEEVNSVEGNSVSITCYYPPT
SVNRHTRKYWCRQGARGGCITLISSEGYVSSKYAGRANLTNFPEN
GTFVVNIAQLSQDDSGRYKCGLGINSRGLSFDVSLEVSQGPGLLN
GSEIVLTQSPGTLSLSPGERATLSCRASQSVSSSYLAWYQQKPGQ
APRLLIYGASSRATGIPDRESGSGSGTETTLTISRLEPEDFAVYY
CQQYGSSTWTFGQGTKVEIKRTVAASGKPEPELVYEDLRGSVTFH
CALGPEVANVAKFLCRQSSGENCDVVVNTLGKRAPAFEGRILLNP
QDKDGSFSVVITGLRKEDAGRYLCGAHSDGQLQEGSPIQAWQLFV
NEESTIPRSPTVVKGVAGSSVAVLCPYNRKESKSIKYWCLWEGAQ
NGRCPLLVDSEGWVKAQYEGRLSLLEEPGNGTFTVILNQLTSRDA
GFYWCLTNGDTLWRTTVEIKIIEGEPNLKVPGNVTAVLGETLKVP
CHFPCKFSSYEKYWCKWNNTGCQALPSQDEGPSKAFVNCDENSRL
VSLTLNLVTRADEGWYWCGVKQGHFYGETAAVYVAVEERGSHHHH
HH
hSC-D2A20.1cdrs-His
(SEQ ID NO: 6)
MLLFVLTCLLAVFPAISTKSPIFGPEEVNSVEGNSVSITCYYPPT
SVNRHTRKYWCRQGARGGCITLISSEGYVSSKYAGRANLTNFPEN
GTFVVNIAQLSQDDSGRYKCGLGINSRGLSFDVSLEVSQGPGLLN
DTKVYTVDLGRTVTINCPFKGRTFSMDPKSLYKQIGLYPVLVIDG
SSTGRTTGYVNPNYTGRIRLDIQGTGQLLFSVVINQLRLSDAGQY
LCAAAPYGANWYRDEYDYKKNADLQVLKPEPELVYEDLRGSVTFH
CALGPEVANVAKFLCRQSSGENCDVVVNTLGKRAPAFEGRILLNP
QDKDGSFSVVITGLRKEDAGRYLCGAHSDGQLQEGSPIQAWQLFV
NEESTIPRSPTVVKGVAGSSVAVLCPYNRKESKSIKYWCLWEGAQ
NGRCPLLVDSEGWVKAQYEGRLSLLEEPGNGTFTVILNQLTSRDA
GFYWCLINGDTLWRTTVEIKIIEGEPNLKVPGNVTAVLGETLKVP
CHFPCKFSSYEKYWCKWNNTGCQALPSQDEGPSKAFVNCDENSRL
VSLTLNLVTRADEGWYWCGVKQGHFYGETAAVYVAVEERGSHHHH
HH
hSC-D2A5.1cdrs-His
(SEQ ID NO: 7)
MLLFVLTCLLAVFPAISTKSPIFGPEEVNSVEGNSVSITCYYPPT
SVNRHTRKYWCRQGARGGCITLISSEGYVSSKYAGRANLTNFPEN
GTFVVNIAQLSQDDSGRYKCGLGINSRGLSFDVSLEVSQGPGLLN
DTKVYTVDLGRTVTINCPFKGRTFSMYRKSLYKQIGLYPVLVIDI
TRNGSSTGYVNPNYTGRIRLDIQGTGQLLFSVVINQLRLSDAGQY
LCAATSGSSYLDAAHVYDYKKNADLQVLKPEPELVYEDLRGSVTF
HCALGPEVANVAKFLCRQSSGENCDVVVNTLGKRAPAFEGRILLN
PQDKDGSFSVVITGLRKEDAGRYLCGAHSDGQLQEGSPIQAWQLF
VNEESTIPRSPTVVKGVAGSSVAVLCPYNRKESKSIKYWCLWEGA
QNGRCPLLVDSEGWVKAQYEGRLSLLEEPGNGTFTVILNQLTSRD
AGFYWCLTNGDTLWRTTVEIKIIEGEPNLKVPGNVTAVLGETLKV
PCHFPCKFSSYEKYWCKWNNTGCQALPSQDEGPSKAFVNCDENSR
LVSLTLNLVTRADEGWYWCGVKQGHFYGETAAVYVAVEERGSHHH
HHH
hSC-ACE2-ala2-His
(SEQ ID NO: 8)
MLLFVLTCLLAVFPAISTKSPIFGPEEVNSVEGNSVSITCYYPPT
SVNRHTRKYWCRQGARGGCITLISSEGYVSSKYAGRANLTNFPEN
GTFVVNIAQLSQDDSGRYKCGLGINSRGLSFDVSLEVSQGPGLLN
GSIEEQAKTFLDKFNHEAEDLFYQSSLASWNYNTNITEENVQNMN
NAGDKWSAFLKEQSTLAQMYPLQEISGKPEPELVYEDLRGSVTFH
CALGPEVANVAKFLCRQSSGENCDVVVNTLGKRAPAFEGRILLNP
QDKDGSFSVVITGLRKEDAGRYLCGAHSDGQLQEGSPIQAWQLFV
NEESTIPRSPTVVKGVAGSSVAVLCPYNRKESKSIKYWCLWEGAQ
NGRCPLLVDSEGWVKAQYEGRLSLLEEPGNGTFTVILNQLTSRDA
GFYWCLTNGDTLWRTTVEIKIIEGEPNLKVPGNVTAVLGETLKVP
CHFPCKFSSYEKYWCKWNNTGCQALPSQDEGPSKAFVNCDENSRL
VSLTLNLVTRADEGWYWCGVKQGHFYGETAAVYVAVEERGSHHHH
HH
hSC-ACE2-ala2-hp-His
(SEQ ID NO: 9)
MLLFVLTCLLAVFPAISTKSPIFGPEEVNSVEGNSVSITCYYPPT
SVNRHTRKYWCRQGARGGCITLISSEGYVSSKYAGRANLTNFPEN
GTFVVNIAQLSQDDSGRYKCGLGINSRGLSFDVSLEVSQGPGLLN
GSIEEQAKTFLDKFNHEAEDLFYQSSLASWNYNTNITEENVQNMN
NAGDKWSAFLKEQSTLAQMYPLQEIQNLTVKLQLQALQQNGSGGG
GGMTQGFWENSMLTDPGNVQKAVCHPTAWDLGKGDFRILMCTSGK
PEPELVYEDLRGSVTFHCALGPEVANVAKFLCRQSSGENCDVVVN
TLGKRAPAFEGRILLNPQDKDGSFSVVITGLRKEDAGRYLCGAHS
DGQLQEGSPIQAWQLFVNEESTIPRSPTVVKGVAGSSVAVLCPYN
RKESKSIKYWCLWEGAQNGRCPLLVDSEGWVKAQYEGRLSLLEEP
GNGTFTVILNQLTSRDAGFYWCLTNGDTLWRTTVEIKIIEGEPNL
KVPGNVTAVLGETLKVPCHFPCKFSSYEKYWCKWNNTGCQALPSQ
DEGPSKAFVNCDENSRLVSLTLNLVTRADEGWYWCGVKQGHFYGE
TAAVYVAVEERGSHHHHHH
hSC-CDB1HC-His
(SEQ ID NO: 10)
MLLFVLTCLLAVFPAISTKSPIFGPEEVNSVEGNSVSITCYYPPT
SVNRHTRKYWCRQGARGGCITLISSEGYVSSKYAGRANLTNFPEN
GTFVVNIAQLSQDDSGRYKCGLGINSRGLSFDVSLEVSQGPGLLN
GSEVQLVQSGAEVKKSGESLKISCKGSGYSFTSYWIGWVRQMPGK
GLEWMGIFYPGDSSTRYSPSFQGQVTISADKSVNTAYLQWSSLKA
SDTAMYYCARRRNWGNAFDIWGQGTMVTVSSSGKPEPELVYEDLR
GSVTFHCALGPEVANVAKFLCRQSSGENCDVVVNTLGKRAPAFEG
RILLNPQDKDGSFSVVITGLRKEDAGRYLCGAHSDGQLQEGSPIQ
AWQLFVNEESTIPRSPTVVKGVAGSSVAVLCPYNRKESKSIKYWC
LWEGAQNGRCPLLVDSEGWVKAQYEGRLSLLEEPGNGTFTVILNQ
LTSRDAGFYWCLTNGDTLWRTTVEIKIIEGEPNLKVPGNVTAVLG
ETLKVPCHFPCKFSSYEKYWCKWNNTGCQALPSQDEGPSKAFVNC
DENSRLVSLTLNLVTRADEGWYWCGVKQGHFYGETAAVYVAVEER
GSHHHHHH
hSC-3D8HC-His
(SEQ ID NO: 11)
MLLFVLTCLLAVFPAISTKSPIFGPEEVNSVEGNSVSITCYYPPT
SVNRHTRKYWCRQGARGGCITLISSEGYVSSKYAGRANLTNFPEN
GTFVVNIAQLSQDDSGRYKCGLGINSRGLSFDVSLEVSQGPGLLN
GSQVQLVESGGGVVQPGRSLRLSCAASGFSFSNYGMHWVRQAPGK
GLEWVALIWYDGSNEDYTDSVKGRFTISRDNSKNTLYLQMNSLRA
EDTAVYYCARWGMVRGVIDVEDIWGQGTVVTVSSSGKPEPELVYE
DLRGSVTFHCALGPEVANVAKFLCRQSSGENCDVVVNTLGKRAPA
FEGRILLNPQDKDGSFSVVITGLRKEDAGRYLCGAHSDGQLQEGS
PIQAWQLFVNEESTIPRSPTVVKGVAGSSVAVLCPYNRKESKSIK
YWCLWEGAQNGRCPLLVDSEGWVKAQYEGRLSLLEEPGNGTFTVI
LNQLTSRDAGFYWCLTNGDTLWRTTVEIKIIEGEPNLKVPGNVTA
VLGETLKVPCHFPCKFSSYEKYWCKWNNTGCQALPSQDEGPSKAF
VNCDENSRLVSLTLNLVTRADEGWYWCGVKQGHFYGETAAVYVAV
EERGSHHHHHH
hSC-3D8VL-His
(SEQ ID NO: 12)
MLLFVLTCLLAVFPAISTKSPIFGPEEVNSVEGNSVSITCYYPPT
SVNRHTRKYWCRQGARGGCITLISSEGYVSSKYAGRANLTNFPEN
GTFVVNIAQLSQDDSGRYKCGLGINSRGLSFDVSLEVSQGPGLLN
GSDIQMTQSPSSVSASVGDRVTITCRASQGISSWLAWYQHKPGKA
PKLLIYAASSLQSGVPSRFSGSGSGTDFTLTISSLQPEDFATYYC
QQANSFPWTFGQGTKVEILGQPKSSSGKPEPELVYEDLRGSVTFH
CALGPEVANVAKFLCRQSSGENCDVVVNTLGKRAPAFEGRILLNP
QDKDGSFSVVITGLRKEDAGRYLCGAHSDGQLQEGSPIQAWQLFV
NEESTIPRSPTVVKGVAGSSVAVLCPYNRKESKSIKYWCLWEGAQ
NGRCPLLVDSEGWVKAQYEGRLSLLEEPGNGTFTVILNQLTSRDA
GFYWCLTNGDTLWRTTVEIKIIEGEPNLKVPGNVTAVLGETLKVP
CHFPCKFSSYEKYWCKWNNTGCQALPSQDEGPSKAFVNCDENSRL
VSLTLNLVTRADEGWYWCGVKQGHFYGETAAVYVAVEERGSHHHH
HH
hSC-4D6VL-His
(SEQ ID NO: 13)
MLLFVLTCLLAVFPAISTKSPIFGPEEVNSVEGNSVSITCYYPPT
SVNRHTRKYWCRQGARGGCITLISSEGYVSSKYAGRANLTNFPEN
GTFVVNIAQLSQDDSGRYKCGLGINSRGLSFDVSLEVSQGPGLLN
QAVVTQESALTTSPGETVTLTCRSSNGAVTSRNYANWVQEKPDHL
FTGLIGGTNNRAPGVPARFSGSLIGDKAALSITGAQTEDEAIYFC
ALWYSNRWVFGGGTKLTVLKPEPELVYEDLRGSVTFHCALGPEVA
NVAKFLCRQSSGENCDVVVNTLGKRAPAFEGRILLNPQDKDGSFS
VVITGLRKEDAGRYLCGAHSDGQLQEGSPIQAWQLFVNEESTIPR
SPTVVKGVAGSSVAVLCPYNRKESKSIKYWCLWEGAQNGRCPLLV
DSEGWVKAQYEGRLSLLEEPGNGTFTVILNQLTSRDAGFYWCLTN
GDTLWRTTVEIKIIEGEPNLKVPGNVTAVLGETLKVPCHFPCKFS
SYEKYWCKWNNTGCQALPSQDEGPSKAFVNCDENSRLVSLTLNLV
TRADEGWYWCGVKQGHFYGETAAVYVAVEERGSHHHHHH
hSC-4D6HC-His
(SEQ ID NO: 14)
MLLFVLTCLLAVFPAISTKSPIFGPEEVNSVEGNSVSITCYYPPT
SVNRHTRKYWCRQGARGGCITLISSEGYVSSKYAGRANLTNFPEN
GTFVVNIAQLSQDDSGRYKCGLGINSRGLSFDVSLEVSQGPGLLN
QVQLQQSDAELVKPGASVKISCKASGYTFTDHAIHWVKQKPEQGL
EWIGYISPGNDDIKYNEKFKGKATLTADTSSSTAYMQLNSLTSED
SAVYFCKVLRRFAYWGQGTLVTVSAKPEPELVYEDLRGSVTFHCA
LGPEVANVAKFLCRQSSGENCDVVVNTLGKRAPAFEGRILLNPQD
KDGSFSVVITGLRKEDAGRYLCGAHSDGQLQEGSPIQAWQLFVNE
ESTIPRSPTVVKGVAGSSVAVLCPYNRKESKSIKYWCLWEGAQNG
RCPLLVDSEGWVKAQYEGRLSLLEEPGNGTFTVILNQLTSRDAGF
YWCLTNGDTLWRTTVEIKIIEGEPNLKVPGNVTAVLGETLKVPCH
FPCKFSSYEKYWCKWNNTGCQALPSQDEGPSKAFVNCDENSRLVS
LTLNLVTRADEGWYWCGVKQGHFYGETAAVYVAVEERGSHHHHHH
hSC-CD46SLP-His
(SEQ ID NO: 15)
MLLFVLTCLLAVFPAISTKSPIFGPEEVNSVEGNSVSITCYYPPT
SVNRHTRKYWCRQGARGGCITLISSEGYVSSKYAGRANLTNFPEN
GTFVVNIAQLSQDDSGRYKCGLGINSRGLSFDVSLEVSQGPGLLN
QVKLEESGGGLVQAGGSLRLSCADSERTFRIYTMAWFRQAPGKER
DFVAAISWSGGSTYYADSVKGRFTISRDNAKNTVYLPMNSLKPDD
TAVYYCASGGVLSTGSQSDSEYDFWGQGTQVTVSSKPEPELVYED
LRGSVTFHCALGPEVANVAKFLCRQSSGENCDVVVNTLGKRAPAF
EGRILLNPQDKDGSFSVVITGLRKEDAGRYLCGAHSDGQLQEGSP
IQAWQLFVNEESTIPRSPTVVKGVAGSSVAVLCPYNRKESKSIKY
WCLWEGAQNGRCPLLVDSEGWVKAQYEGRLSLLEEPGNGTFTVIL
NQLTSRDAGFYWCLTNGDTLWRTTVEIKIIEGEPNLKVPGNVTAV
LGETLKVPCHFPCKFSSYEKYWCKWNNTGCQALPSQDEGPSKAFV
NCDENSRLVSLTLNLVTRADEGWYWCGVKQGHFYGETAAVYVAVE
ERGSHHHHHH
hSC-6G9VL-His
(SEQ ID NO: 16)
MLLFVLTCLLAVFPAISTKSPIFGPEEVNSVEGNSVSITCYYPPT
SVNRHTRKYWCRQGARGGCITLISSEGYVSSKYAGRANLTNFPEN
GTFVVNIAQLSQDDSGRYKCGLGINSRGLSFDVSLEVSQGPGLLN
DIVLTQSPASLAVSLGQRATISCRASKSVSTSGYSYMHWYQQKPG
QPPKLLIYLASNLESGVPARFSGSGSGTDFTLNIHPVEEEDAATY
YCQHSRELPRTFGGGTKLEIKKPEPELVYEDLRGSVTFHCALGPE
VANVAKFLCRQSSGENCDVVVNTLGKRAPAFEGRILLNPQDKDGS
FSVVITGLRKEDAGRYLCGAHSDGQLQEGSPIQAWQLFVNEESTI
PRSPTVVKGVAGSSVAVLCPYNRKESKSIKYWCLWEGAQNGRCPL
LVDSEGWVKAQYEGRLSLLEEPGNGTFTVILNQLTSRDAGFYWCL
TNGDTLWRTTVEIKIIEGEPNLKVPGNVTAVLGETLKVPCHFPCK
FSSYEKYWCKWNNTGCQALPSQDEGPSKAFVNCDENSRLVSLTLN
LVTRADEGWYWCGVKQGHFYGETAAVYVAVEERGSHHHHHH
hSC-6G9HC-His
(SEQ ID NO: 17)
MLLFVLTCLLAVFPAISTKSPIFGPEEVNSVEGNSVSITCYYPPT
SVNRHTRKYWCRQGARGGCITLISSEGYVSSKYAGRANLTNFPEN
GTFVVNIAQLSQDDSGRYKCGLGINSRGLSFDVSLEVSQGPGLLN
EVQLQQSGPELVKPGASVKISCKASGYTFTDYNMWVKQSHGKSLE
WIGYIYPYNGGTGYNQKFKSKATLTVDNSSSTAYMELRSLTSEDS
AVYYCARNYYGSSWFAYWGQGTLVTVSAKPEPELVYEDLRGSVTF
HCALGPEVANVAKFLCRQSSGENCDVVVNTLGKRAPAFEGRILLN
PQDKDGSFSVVITGLRKEDAGRYLCGAHSDGQLQEGSPIQAWQLF
VNEESTIPRSPTVVKGVAGSSVAVLCPYNRKESKSIKYWCLWEGA
QNGRCPLLVDSEGWVKAQYEGRLSLLEEPGNGTFTVILNQLTSRD
AGFYWCLINGDTLWRTTVEIKIIEGEPNLKVPGNVTAVLGETLKV
PCHFPCKFSSYEKYWCKWNNTGCQALPSQDEGPSKAFVNCDENSR
LVSLTLNLVTRADEGWYWCGVKQGHFYGETAAVYVAVEERGSHHH
HHH
Mouse secretory component (mSC)-A20.1-His
(SEQ ID NO: 18)
MRLYLFTLLVTVFSGVSTKSPIFGPQEVSSIEGDSVSITCYYPDT
SVNRHTRKYWCRQGASGMCTTLISSNGYLSKEYSGRANLINFPEN
NTFVINIEQLTQDDTGSYKCGLGTSNRGLSFDVSLEVSQVPELPS
QVQLVESGGGLAQAGGSLRLSCAASGRTFSMDPMAWFRQPPGKER
EFVAAGSSTGRTTYYADSVKGRFTISRDNAKNTVYLQMNSLKPED
TAVYYCAAAPYGANWYRDEYDYWGQGTQVTVSSAPEPELLYKDLR
SSVTFECDLGREVANEAKYLCRMNKETCDVIINTLGKRDPDFEGR
ILITPKDDNGRFSVLITGLRKEDAGHYQCGAHSSGLPQEGWPIQT
WQLFVNEESTIPNRRSVVKGVTGGSVAIACPYNPKESSSLKYWCR
WEGDGNGHCPVLVGTQAQVQEEYEGRLALFDQPGNGTYTVILNQL
TTEDAGFYWCLTNGDSRWRTTIELQVAEATREPNLEVTPQNATAV
LGETFTVSCHYPCKFYSQEKYWCKWSNKGCHILPSHDEGARQSSV
SCDQSSQLVSMTLNPVSKEDEGWYWCGVKQGQTYGETTAIYIAVE
ERGSHHHHHH
mSC-A5.1-His
(SEQ ID NO: 19)
MRLYLFTLLVTVFSGVSTKSPIFGPQEVSSIEGDSVSITCYYPDT
SVNRHTRKYWCRQGASGMCTTLISSNGYLSKEYSGRANLINFPEN
NTFVINIEQLTQDDTGSYKCGLGTSNRGLSFDVSLEVSQVPELPS
QVKLEESGGGLVQAGGSLRLSCAASGRTFSMYRMGWFRQAPGKER
EFVGVITRNGSSTYYADSVKGRFTISRDNAKNTVYLQMNSLKPED
TALYYCAATSGSSYLDAAHVYDYWGQGTQVTVSSAPEPELLYKDL
RSSVTFECDLGREVANEAKYLCRMNKETCDVIINTLGKRDPDFEG
RILITPKDDNGRFSVLITGLRKEDAGHYQCGAHSSGLPQEGWPIQ
TWQLFVNEESTIPNRRSVVKGVTGGSVAIACPYNPKESSSLKYWC
RWEGDGNGHCPVLVGTQAQVQEEYEGRLALFDQPGNGTYTVILNQ
LTTEDAGFYWCLTNGDSRWRTTIELQVAEATREPNLEVTPQNATA
VLGETFTVSCHYPCKFYSQEKYWCKWSNKGCHILPSHDEGARQSS
VSCDQSSQLVSMTLNPVSKEDEGWYWCGVKQGQTYGETTAIYIAV
EERGSHHHHHH
mSC-CD5SLP-His
(SEQ ID NO: 20)
MRLYLFTLLVTVFSGVSTKSPIFGPQEVSSIEGDSVSITCYYPDT
SVNRHTRKYWCRQGASGMCTTLISSNGYLSKEYSGRANLINFPEN
NTFVINIEQLTQDDTGSYKCGLGTSNRGLSFDVSLEVSQVPELPS
QVKLEESGGGLVQAGGSLRLSCAASRLTESTYHMGWFRQAPGKER
EFVAALSWSGGTTYYADSVKGRFGISRDNAKNTVYLQMNSLKPED
TAVYYCASGGVLATMNSDEYDYWGQGTQVTVSSAPEPELLYKDLR
SSVTFECDLGREVANEAKYLCRMNKETCDVIINTLGKRDPDFEGR
ILITPKDDNGRFSVLITGLRKEDAGHYQCGAHSSGLPQEGWPIQT
WQLFVNEESTIPNRRSVVKGVTGGSVAIACPYNPKESSSLKYWCR
WEGDGNGHCPVLVGTQAQVQEEYEGRLALFDQPGNGTYTVILNQL
TTEDAGFYWCLTNGDSRWRTTIELQVAEATREPNLEVTPQNATAV
LGETFTVSCHYPCKFYSQEKYWCKWSNKGCHILPSHDEGARQSSV
SCDQSSQLVSMTLNPVSKEDEGWYWCGVKQGQTYGETTAIYIAVE
ERGSHHHHHH
mSC-CDB1-His
(SEQ ID NO: 21)
MRLYLFTLLVTVFSGVSTKSPIFGPQEVSSIEGDSVSITCYYPDT
SVNRHTRKYWCRQGASGMCTTLISSNGYLSKEYSGRANLINFPEN
NTFVINIEQLTQDDTGSYKCGLGTSNRGLSFDVSLEVSQVPELPS
EIVLTQSPGTLSLSPGERATLSCRASQSVSSSYLAWYQQKPGQAP
RLLIYGASSRATGIPDRESGSGSGTETTLTISRLEPEDFAVYYCQ
QYGSSTWTFGQGTKVEIKAPEPELLYKDLRSSVTFECDLGREVAN
EAKYLCRMNKETCDVIINTLGKRDPDFEGRILITPKDDNGRFSVL
ITGLRKEDAGHYQCGAHSSGLPQEGWPIQTWQLFVNEESTIPNRR
SVVKGVTGGSVAIACPYNPKESSSLKYWCRWEGDGNGHCPVLVGT
QAQVQEEYEGRLALFDQPGNGTYTVILNQLTTEDAGFYWCLTNGD
SRWRTTIELQVAEATREPNLEVTPQNATAVLGETFTVSCHYPCKF
YSQEKYWCKWSNKGCHILPSHDEGARQSSVSCDQSSQLVSMTLNP
VSKEDEGWYWCGVKQGQTYGETTAIYIAVEERGSHHHHHH
mSC-CDB1HC-His
(SEQ ID NO: 22)
MRLYLFTLLVTVFSGVSTKSPIFGPQEVSSIEGDSVSITCYYPDT
SVNRHTRKYWCRQGASGMCTTLISSNGYLSKEYSGRANLINFPEN
NTFVINIEQLTQDDTGSYKCGLGTSNRGLSFDVSLEVSQVPELPS
EVQLVQSGAEVKKSGESLKISCKGSGYSFTSYWIGWVRQMPGKGL
EWMGIFYPGDSSTRYSPSFQGQVTISADKSVNTAYLQWSSLKASD
TAMYYCARRRNWGNAFDIWGQGTMVTVSSAPEPELLYKDLRSSVT
FECDLGREVANEAKYLCRMNKETCDVIINTLGKRDPDFEGRILIT
PKDDNGRFSVLITGLRKEDAGHYQCGAHSSGLPQEGWPIQTWQLF
VNEESTIPNRRSVVKGVTGGSVAIACPYNPKESSSLKYWCRWEGD
GNGHCPVLVGTQAQVQEEYEGRLALFDQPGNGTYTVILNQLTTED
AGFYWCLINGDSRWRTTIELQVAEATREPNLEVTPQNATAVLGET
FTVSCHYPCKFYSQEKYWCKWSNKGCHILPSHDEGARQSSVSCDQ
SSQLVSMTLNPVSKEDEGWYWCGVKQGQTYGETTAIYIAVEERGS
HHHHHH
mSC-3D8HC-His
(SEQ ID NO: 23)
MRLYLFTLLVTVFSGVSTKSPIFGPQEVSSIEGDSVSITCYYPDT
SVNRHTRKYWCRQGASGMCTTLISSNGYLSKEYSGRANLINFPEN
NTFVINIEQLTQDDTGSYKCGLGTSNRGLSFDVSLEVSQVPELPS
QVQLVESGGGVVQPGRSLRLSCAASGFSFSNYGMHWVRQAPGKGL
EWVALIWYDGSNEDYTDSVKGRFTISRDNSKNTLYLQMNSLRAED
TAVYYCARWGMVRGVIDVEDIWGQGTVVTVSSAPEPELLYKDLRS
SVTFECDLGREVANEAKYLCRMNKETCDVIINTLGKRDPDFEGRI
LITPKDDNGRFSVLITGLRKEDAGHYQCGAHSSGLPQEGWPIQTW
QLFVNEESTIPNRRSVVKGVTGGSVAIACPYNPKESSSLKYWCRW
EGDGNGHCPVLVGTQAQVQEEYEGRLALFDQPGNGTYTVILNQLT
TEDAGFYWCLINGDSRWRTTIELQVAEATREPNLEVTPQNATAVL
GETFTVSCHYPCKFYSQEKYWCKWSNKGCHILPSHDEGARQSSVS
CDQSSQLVSMTLNPVSKEDEGWYWCGVKQGQTYGETTAIYIAVEE
RGSHHHHHH
mSC-3D8VL-His
(SEQ ID NO: 24)
MRLYLFTLLVTVFSGVSTKSPIFGPQEVSSIEGDSVSITCYYPDT
SVNRHTRKYWCRQGASGMCTTLISSNGYLSKEYSGRANLINFPEN
NTFVINIEQLTQDDTGSYKCGLGTSNRGLSFDVSLEVSQVPELPS
DIQMTQSPSSVSASVGDRVTITCRASQGISSWLAWYQHKPGKAPK
LLIYAASSLQSGVPSRFSGSGSGTDFTLTISSLQPEDFATYYCQQ
ANSFPWTFGQGTKVEIKAPEPELLYKDLRSSVTFECDLGREVANE
AKYLCRMNKETCDVIINTLGKRDPDFEGRILITPKDDNGRFSVLI
TGLRKEDAGHYQCGAHSSGLPQEGWPIQTWQLFVNEESTIPNRRS
VVKGVTGGSVAIACPYNPKESSSLKYWCRWEGDGNGHCPVLVGTQ
AQVQEEYEGRLALFDQPGNGTYTVILNQLTTEDAGFYWCLTNGDS
RWRTTIELQVAEATREPNLEVTPQNATAVLGETFTVSCHYPCKFY
SQEKYWCKWSNKGCHILPSHDEGARQSSVSCDQSSQLVSMTLNPV
SKEDEGWYWCGVKQGQTYGETTAIYIAVEERGSHHHHHH
mSC-4D6VL-His
(SEQ ID NO: 25)
MRLYLFTLLVTVFSGVSTKSPIFGPQEVSSIEGDSVSITCYYPDT
SVNRHTRKYWCRQGASGMCTTLISSNGYLSKEYSGRANLINFPEN
NTFVINIEQLTQDDTGSYKCGLGTSNRGLSFDVSLEVSQVPELPS
QAVVTQESALTTSPGETVTLTCRSSNGAVTSRNYANWVQEKPDHL
FTGLIGGTNNRAPGVPARESGSLIGDKAALSITGAQTEDEAIYFC
ALWYSNRWVFGGGTKLTVLAPEPELLYKDLRSSVTFECDLGREVA
NEAKYLCRMNKETCDVIINTLGKRDPDFEGRILITPKDDNGRFSV
LITGLRKEDAGHYQCGAHSSGLPQEGWPIQTWQLFVNEESTIPNR
RSVVKGVTGGSVAIACPYNPKESSSLKYWCRWEGDGNGHCPVLVG
TQAQVQEEYEGRLALFDQPGNGTYTVILNQLTTEDAGFYWCLTNG
DSRWRTTIELQVAEATREPNLEVTPQNATAVLGETFTVSCHYPCK
FYSQEKYWCKWSNKGCHILPSHDEGARQSSVSCDQSSQLVSMTLN
PVSKEDEGWYWCGVKQGQTYGETTAIYIAVEERGSHHHHHH
mSC-4D6HC-His
(SEQ ID NO: 26)
MRLYLFTLLVTVFSGVSTKSPIFGPQEVSSIEGDSVSITCYYPDT
SVNRHTRKYWCRQGASGMCTTLISSNGYLSKEYSGRANLINFPEN
NTFVINIEQLTQDDTGSYKCGLGTSNRGLSFDVSLEVSQVPELPS
QVQLQQSDAELVKPGASVKISCKASGYTFTDHAIHWVKQKPEQGL
EWIGYISPGNDDIKYNEKFKGKATLTADTSSSTAYMQLNSLTSED
SAVYFCKVLRRFAYWGQGTLVTVSAAPEPELLYKDLRSSVTFECD
LGREVANEAKYLCRMNKETCDVIINTLGKRDPDFEGRILITPKDD
NGRFSVLITGLRKEDAGHYQCGAHSSGLPQEGWPIQTWQLFVNEE
STIPNRRSVVKGVTGGSVAIACPYNPKESSSLKYWCRWEGDGNGH
CPVLVGTQAQVQEEYEGRLALFDQPGNGTYTVILNQLTTEDAGFY
WCLTNGDSRWRTTIELQVAEATREPNLEVTPQNATAVLGETFTVS
CHYPCKFYSQEKYWCKWSNKGCHILPSHDEGARQSSVSCDQSSQL
VSMTLNPVSKEDEGWYWCGVKQGQTYGETTAIYIAVEERGSHHHH
HH
mSC-CD46SLP-His
(SEQ ID NO: 27)
MRLYLFTLLVTVFSGVSTKSPIFGPQEVSSIEGDSVSITCYYPDT
SVNRHTRKYWCRQGASGMCTTLISSNGYLSKEYSGRANLINFPEN
NTFVINIEQLTQDDTGSYKCGLGTSNRGLSFDVSLEVSQVPELPS
QVKLEESGGGLVQAGGSLRLSCADSERTFRIYTMAWFRQAPGKER
DFVAAISWSGGSTYYADSVKGRFTISRDNAKNTVYLPMNSLKPDD
TAVYYCASGGVLSTGSQSDSEYDFWGQGTQVTVSSAPEPELLYKD
LRSSVTFECDLGREVANEAKYLCRMNKETCDVIINTLGKRDPDFE
GRILITPKDDNGRFSVLITGLRKEDAGHYQCGAHSSGLPQEGWPI
QTWQLFVNEESTIPNRRSVVKGVTGGSVAIACPYNPKESSSLKYW
CRWEGDGNGHCPVLVGTQAQVQEEYEGRLALFDQPGNGTYTVILN
QLTTEDAGFYWCLTNGDSRWRTTIELQVAEATREPNLEVTPQNAT
AVLGETFTVSCHYPCKFYSQEKYWCKWSNKGCHILPSHDEGARQS
SVSCDQSSQLVSMTLNPVSKEDEGWYWCGVKQGQTYGETTAIYIA
VEERGSHHHHHH
mSC-6G9VL-His
(SEQ ID NO: 28)
MRLYLFTLLVTVFSGVSTKSPIFGPQEVSSIEGDSVSITCYYPDT
SVNRHTRKYWCRQGASGMCTTLISSNGYLSKEYSGRANLINFPEN
NTFVINIEQLTQDDTGSYKCGLGTSNRGLSFDVSLEVSQVPELPS
DIVLTQSPASLAVSLGQRATISCRASKSVSTSGYSYMHWYQQKPG
QPPKLLIYLASNLESGVPARFSGSGSGTDFTLNIHPVEEEDAATY
YCQHSRELPRTFGGGTKLEIKAPEPELLYKDLRSSVTFECDLGRE
VANEAKYLCRMNKETCDVIINTLGKRDPDFEGRILITPKDDNGRF
SVLITGLRKEDAGHYQCGAHSSGLPQEGWPIQTWQLFVNEESTIP
NRRSVVKGVTGGSVAIACPYNPKESSSLKYWCRWEGDGNGHCPVL
VGTQAQVQEEYEGRLALFDQPGNGTYTVILNQLTTEDAGFYWCLI
NGDSRWRTTIELQVAEATREPNLEVTPQNATAVLGETFTVSCHYP
CKFYSQEKYWCKWSNKGCHILPSHDEGARQSSVSCDQSSQLVSMT
LNPVSKEDEGWYWCGVKQGQTYGETTAIYIAVEERGSHHHHHH
mSC-6G9HC-His
(SEQ ID NO: 29)
MRLYLFTLLVTVFSGVSTKSPIFGPQEVSSIEGDSVSITCYYPDT
SVNRHTRKYWCRQGASGMCTTLISSNGYLSKEYSGRANLINFPEN
NTFVINIEQLTQDDTGSYKCGLGTSNRGLSFDVSLEVSQVPELPS
EVQLQQSGPELVKPGASVKISCKASGYTFTDYNMWVKQSHGKSLE
WIGYIYPYNGGTGYNQKFKSKATLTVDNSSSTAYMELRSLTSEDS
AVYYCARNYYGSSWFAYWGQGTLVTVSAAPEPELLYKDLRSSVTF
ECDLGREVANEAKYLCRMNKETCDVIINTLGKRDPDFEGRILITP
KDDNGRFSVLITGLRKEDAGHYQCGAHSSGLPQEGWPIQTWQLFV
NEESTIPNRRSVVKGVTGGSVAIACPYNPKESSSLKYWCRWEGDG
NGHCPVLVGTQAQVQEEYEGRLALFDQPGNGTYTVILNQLTTEDA
GFYWCLINGDSRWRTTIELQVAEATREPNLEVTPQNATAVLGETF
TVSCHYPCKFYSQEKYWCKWSNKGCHILPSHDEGARQSSVSCDQS
SQLVSMTLNPVSKEDEGWYWCGVKQGQTYGETTAIYIAVEERGSH
HHHHH
hSC-SD36-His
(SEQ ID NO: 114)
MLLFVLTCLLAVFPAISTKSPIFGPEEVNSVEGNSVSITCYYPPT
SVNRHTRKYWCRQGARGGCITLISSEGYVSSKYAGRANLTNFPEN
GTFVVNIAQLSQDDSGRYKCGLGINSRGLSFDVSLEVSQGPGLLN
GSEVQLVESGGGLVQAGGSLKLSCAASGRTYAMGWFRQAPGKERE
FVAHINALGTRTYYSDSVKGRFTISRDNAKNTEYLEMNNLKPEDT
AVYYCTAQGQWRAAPVAVAAEYEFWGQGTQVTVSSGKPEPELVYE
DLRGSVTFHCALGPEVANVAKFLCRQSSGENCDVVVNTLGKRAPA
FEGRILLNPQDKDGSFSVVITGLRKEDAGRYLCGAHSDGQLQEGS
PIQAWQLFVNEESTIPRSPTVVKGVAGSSVAVLCPYNRKESKSIK
YWCLWEGAQNGRCPLLVDSEGWVKAQYEGRLSLLEEPGNGTFTVI
LNQLTSRDAGFYWCLTNGDTLWRTTVEIKIIEGEPNLKVPGNVTA
VLGETLKVPCHFPCKFSSYEKYWCKWNNTGCQALPSQDEGPSKAF
VNCDENSRLVSLTLNLVTRADEGWYWCGVKQGHFYGETAAVYVAV
EERGSHHHHHH
hSC-SD38-His
(SEQ ID NO: 115)
MLLFVLTCLLAVFPAISTKSPIFGPEEVNSVEGNSVSITCYYPPT
SVNRHTRKYWCRQGARGGCITLISSEGYVSSKYAGRANLTNFPEN
GTFVVNIAQLSQDDSGRYKCGLGINSRGLSFDVSLEVSQGPGLLN
GSEVQLVESGGGLVQPGGSLRLSCAVSISIFDIYAMDWYRQAPGK
QRDLVATSFRDGSTNYADSVKGRFTISRDNAKNTLYLQMNSLKPE
DTAVYLCHVSLYRDPLGVAGGMGVYWGKGALVTVSSSGKPEPELV
YEDLRGSVTFHCALGPEVANVAKFLCRQSSGENCDVVVNTLGKRA
PAFEGRILLNPQDKDGSFSVVITGLRKEDAGRYLCGAHSDGQLQE
GSPIQAWQLFVNEESTIPRSPTVVKGVAGSSVAVLCPYNRKESKS
IKYWCLWEGAQNGRCPLLVDSEGWVKAQYEGRLSLLEEPGNGTFT
VILNQLTSRDAGFYWCLTNGDTLWRTTVEIKIIEGEPNLKVPGNV
TAVLGETLKVPCHFPCKFSSYEKYWCKWNNTGCQALPSQDEGPSK
AFVNCDENSRLVSLTLNLVTRADEGWYWCGVKQGHFYGETAAVYV
AVEERGSHHHHHH

TABLE 1
Modified SC proteins

| Expression | SEQ ID | | | | | |
|---|---|---|---|---|---|---|
| construct name | NO: | Module name | Type | Species specificity | Strain specificity | Antigen specificity |
| hSC-A20.1-His | 2 | A20.1 | sdAb | Clostridium difficile | VPI 10463 | TcdA (amino acid |
| | | | | | | residues 2304-2710) |
| hSC-A5.1-His | 3 | A5.1 | sdAb | Clostridium difficile | VPI 10463 | TcdA (amino acid |
| | | | | | | residues 2304-2710) |
| hSC-CD5SLP-His | 4 | VHH5 | sdAb | Clostridium difficile | Strains QCD-32g58 | LMW subunit of the |
| | | | | | (GenBank Acc. No. | SLP |
| | | | | | AAML00000000) | |
| hSC-CDB1-His | 5 | 124-152 (VL) | Fab-VL | Clostridium difficile | VPI 10463 | TcdB-RBD |
| hSC- | 6 | A20 CDRs | CDR substitution | Clostridium difficile | VPI 10463 | TcdA (amino acid |
| D2A20.1cdrs-His | | | | | | residues 2304-2710) |
| hSC-D2A5.1cdrs- | 7 | A5 CDRs | CDR substitution | Clostridium difficile | VPI 10463 | TcdA (amino acid |
| His | | | | | | residues 2304-2710) |
| hSC-ACE2-ala2- | 8 | ACE2- | peptide with 2 | Homo sapiens | SARS-CoV-2 | S protein RBD |
| His | | peptide1 | helices | | | |
| hSC-ACE2-ala2- | 9 | ACE2- | peptide with 2 | Homo sapiens | SARS-CoV-2 | S protein RBD |
| hp-His | | peptide2 | helices and a | | | |
| | | | beta hairpin | | | |
| hSC-CDB1HC- | 10 | 124-152 (VH) | Fab-VH | Clostridium difficile | VPI 10463 | TcdB-RBD |
| His | | | | | | |
| hSC-3D8HC-His | 11 | 3D8 (VH) | Fab-VH | Clostridium difficile | VPI 10463 | TcdA |
| hSC-3D8VL-His | 12 | 3D8 (VL) | Fab-VL | Clostridium difficile | VPI 10463 | TcdA |
| hSC-4D6VL-His | 13 | 4D6 (VL) | Fab-VL | Clostridium difficile | Cd630 | Lipothechoic acid |
| hSC-4D6HC-His | 14 | 4D6 (VH) | Fab-VH | Clostridium difficile | Cd630 | Lipothechoic acid |
| hSC-CD46SLP- | 15 | VHH46 | sdAb | Clostridium difficile | Strains QCD-32g58 | LMW subunit of the |
| His | | | | | (GenBank Acc. No. | SLP |
| | | | | | AAML00000000) | |
| hSC-6G9VL-His | 16 | 6G9 (VL) | Fab-VL | Clostridium difficile | Cd630 | Lipothechoic acid |
| hSC-6G9HC-His | 17 | 6G9 (VH) | Fab-VH | Clostridium difficile | Cd630 | Lipothechoic acid |
| mSC-A20.1-His | 18 | A20.1 | sdAb | Clostridium difficile | VPI 10463 | TcdA (amino acid |
| | | | | | | residues 2304-2710) |
| mSC-A5.1-His | 19 | A5.1 | sdAb | Clostridium difficile | VPI 10463 | TcdA (amino acid |
| | | | | | | residues 2304-2710) |
| mSC-CD5SLP- | 20 | VHH5 | sdAb | Clostridium difficile | Strains QCD-32g58 | LMW subunit of the |
| His | | | | | (GenBank Acc. No. | SLP |
| | | | | | AAML00000000) | |
| mSC-CDB1-His | 21 | 124-152 (VL) | Fab-VL | Clostridium difficile | VPI 10463 | TcdB-RBD |
| mSC-CDB1HC- | 22 | 124-152 (VH) | Fab-VH | Clostridium difficile | VPI 10463 | TcdB-RBD |
| His | | | | | | |
| mSC-3D8HC-His | 23 | 3D8 (VH) | Fab-VH | Clostridium difficile | VPI 10463 | TcdA |
| mSC-3D8VL-His | 24 | 3D8 (VL) | Fab-VL | Clostridium difficile | VPI 10463 | TcdA |
| mSC-4D6VL-His | 25 | 4D6 (VL) | Fab-VL | Clostridium difficile | Cd630 | Lipothechoic acid |
| mSC-4D6HC-His | 26 | 4D6 (VH) | Fab-VH | Clostridium difficile | Cd630 | Lipothechoic acid |
| mSC-CD46SLP- | 27 | VHH46 | sdAb | Clostridium difficile | Strains QCD-32g58 | LMW subunit of the |
| His | | | | | (GenBank Acc. No. | SLP |
| | | | | | AAML00000000) | |
| mSC-6G9VL-His | 28 | 6G9 (VL) | Fab-VL | Clostridium difficile | Cd630 | Lipothechoic acid |
| mSC-6G9HC-His | 29 | 6G9 (VH) | Fab-VH | Clostridium difficile | Cd630 | Lipothechoic acid |
| hSC-SD36-His | 114 | SD36 | sdAb | Influenza type A | Group 2 (H3, H4, H7, | Hemagglutinin (HA) |
| | | | | | H10) | |
| hSC-SD38-His | 115 | SD38 | sdAb | Influenza type A | Group 1 (H1, H2, H5) | Hemagglutinin (HA) |
| | | | | | Group 2 (H3, H7, H10) | |

TABLE 2
Exemplary antibody sequences for replacement of the D2 domain

| | | | | | | SEQ |
|---|---|---|---|---|---|---|
| Module | | Species | Strain | Antigen | | ID |
| Name | Type | Specificity | specificity | specificity | Antibody Sequence | NO: |
| A20.1 | sdAb | Clostridium | VPI 10463 | TcdA (amino acid | QVQLVESGGGLAQAGGSLRLSCAAS | 30 |
| | | difficile | | residues 2304-2710) | GRTFSMDPMAWFRQPPGKEREFVAA | |
| | | | | | GSSTGRTTYYADSVKGRFTISRDNAK | |
| | | | | | NTVYLQMNSLKPEDTAVYYCAAAPY | |
| | | | | | GANWYRDEYDYWGQGTQVTVSS | |
| A5.1 | sdAb | Clostridium | VPI 10463 | TcdA (amino acid | QVKLEESGGGLVQAGGSLRLSCAASG | 31 |
| | | difficile | | residues 2304-2710) | RTFSMYRMGWFRQAPGKEREFVGVI | |
| | | | | | TRNGSSTYYADSVKGRFTISRDNAKN | |
| | | | | | TVYLQMNSLKPEDTALYYCAATSGSS | |
| | | | | | YLDAAHVYDYWGQGTQVTVSS | |
| VHH5 | sdAb | Clostridium | Strains | LMW subunit of the | QVKLEESGGGLVQAGGSLRLSCAASR | 32 |
| | | difficile | QCD-32g58 | SLP | LTFSTYHMGWFRQAPGKEREFVAAL | |
| | | | (GenBank | | SWSGGTTYYADSVKGRFGISRDNAK | |
| | | | Acc. No. | | NTVYLQMNSLKPEDTAVYYCASGGV | |
| | | | AAML00000000) | | LATMNSDEYDYWGQGTQVTVSS | |
| VHH46 | sdAb | Clostridium | Strains | LMW subunit of the | QVKLEESGGGLVQAGGSLRLSCADSE | 33 |
| | | difficile | QCD-32g58 | SLP | RTFRIYTMAWFRQAPGKERDFVAAIS | |
| | | | (GenBank | | WSGGSTYYADSVKGRFTISRDNAKNT | |
| | | | Acc. No. | | VYLPMNSLKPDDTAVYYCASGGVLS | |
| | | | AAML00000000) | | TGSQSDSEYDFWGQGTQVTVSS | |
| 124-152 | Fab-VH | Clostridium | VPI 10463 | TcdB-RBD | EVQLVQSGAEVKKSGESLKISCKGSG | 34 |
| (VH) | | difficile | | | YSFTSYWIGWVRQMPGKGLEWMGIF | |
| | | | | | YPGDSSTRYSPSFQGQVTISADKSVNT | |
| | | | | | AYLQWSSLKASDTAMYYCARRRNW | |
| | | | | | GNAFDIWGQGTMVTVSS | |
| 124-152 | Fab-VL | Clostridium | VPI 10463 | TcdB-RBD | EIVLTQSPGTLSLSPGERATLSCRASQS | 35 |
| (VL) | | difficile | | | VSSSYLAWYQQKPGQAPRLLIYGASS | |
| | | | | | RATGIPDRFSGSGSGTETTLTISRLEPE | |
| | | | | | DFAVYYCQQYGSSTWTFGQGTKVEI | |
| | | | | | K | |
| 3D8 (VH) | Fab-VH | Clostridium | VPI 10463 | TcdA | QVQLVESGGGVVQPGRSLRLSCAASG | 36 |
| | | difficile | | | FSFSNYGMHWVRQAPGKGLEWVALI | |
| | | | | | WYDGSNEDYTDSVKGRFTISRDNSKN | |
| | | | | | TLYLQMNSLRAEDTAVYYCARWGM | |
| | | | | | VRGVIDVFDIWGQGTVVTVSS | |
| 3D8 (VL) | Fab-VL | Clostridium | VPI 10463 | TcdA | DIQMTQSPSSVSASVGDRVTITCRASQ | 37 |
| | | difficile | | | GISSWLAWYQHKPGKAPKLLIYAASS | |
| | | | | | LQSGVPSRFSGSGSGTDFTLTISSLQPE | |
| | | | | | DFATYYCQQANSFPWTFGQGTKVEIK | |
| PA41 (VL) | Fab-VL | Clostridium | Strain R20291 | TcdB | EIVLTQSPATLSLSPGERATLSCRASQS | 38 |
| | | difficile | | | VGTSIHWYQQKPGQAPRLLIKFASESI | |
| | | | | | SGIPARFSGSGSGTDFTLTISSLEPEDF | |
| | | | | | AVYYCQQSNKWPFTFGQGTKLEIKRT | |
| PA50 (VL) | Fab-VL | Clostridium | | TcdA | EIVLTQSPATLSLSPGERATLSCRASSS | 39 |
| | | difficile | | | VNYMNWYQQKPGQAPRPLIYATSNL | |
| | | | | | ASGIPARFSGSGSGTDFTLTISSLEPED | |
| | | | | | FAVYYCQQWSSRTFGGGTKLEIKRT | |
| 4D6VH | Fab-VH | Clostridium | Cd630 | Lipothechoic acid | QVQLQQSDAELVKPGASVKISCKASG | 40 |
| | | difficile | | | YTFTDHAIHWVKQKPEQGLEWIGYIS | |
| | | | | | PGNDDIKYNEKFKGKATLTADTSSST | |
| | | | | | AYMQLNSLTSEDSAVYFCKVLRRFA | |
| | | | | | YWGQGTLVTVSA | |
| 4D6VL | Fab-VL | Clostridium | Cd630 | Lipothechoic acid | QAVVTQESALTTSPGETVTLTCRSSN | 41 |
| | | difficile | | | GAVTSRNYANWVQEKPDHLFTGLIG | |
| | | | | | GTNNRAPGVPARFSGSLIGDKAALSIT | |
| | | | | | GAQTEDEAIYFCALWYSNRWVFGGG | |
| | | | | | TKLTVL | |
| 6G9VH | Fab-VH | Clostridium | Cd630 | Lipothechoic acid | EVQLQQSGPELVKPGASVKISCKASG | 42 |
| | | difficile | | | YTFTDYNMWVKQSHGKSLEWIGYIY | |
| | | | | | PYNGGTGYNQKFKSKATLTVDNSSST | |
| | | | | | AYMELRSLTSEDSAVYYCARNYYGS | |
| | | | | | SWFAYWGQGTLVTVSA | |
| 6G9VL | Fab-VL | Clostridium | Cd630 | Lipothechoic acid | DIVLTQSPASLAVSLGQRATISCRASK | 43 |
| | | difficile | | | SVSTSGYSYMHWYQQKPGQPPKLLIY | |
| | | | | | LASNLESGVPARFSGSGSGTDFTLNIH | |
| | | | | | PVEEEDAATYYCQHSRELPRTFGGGT | |
| | | | | | KLEIK | |
| B39 | sdAb | Clostridium | VPI 10463 | TcdB-RBD | QVQLVESGGGLVQAGGSLRLSCAAS | 44 |
| | | difficile | | | GLTFSRYVMGWFRQAPGKEREFVAAI | |
| | | | | | TWGGTPNYADSVKGRFTISRDNSKNT | |
| | | | | | QYLQMNSLKPEDTAVYYCAAGLGW | |
| | | | | | DSRYSQSYNYWGQGTQVTVSSGSEQ | |
| | | | | | KLISEEDLNHHHHHH | |
| 1B11VH | Fab-VH | Clostridium | VPI 10463 | TcdA-GTD | QMQLVESGGGVVQPGRSLRLSCEAS | 45 |
| | | difficile | | | GFSFNSYGMHWVRQAPGKGLEWVS | |
| | | | | | VIWASGNKKYYIESVEGRFTISRDNSK | |
| | | | | | NTLYLQMNSLRAEDTAVYYCARANF | |
| | | | | | DYWGQGTLVTVSS | |
| 1B11VL | Fab-VL | Clostridium | VPI 10463 | TcdA-GTD | EIVLTQSPATLSLSPGERATLSCRASQS | 46 |
| | | difficile | | | VSSYLAWYQQKPGQAPRLLIYDASNR | |
| | | | | | ATGIPARFSGSGSGTDFTLTISSLEPED | |
| | | | | | FAVYYCQQRSNWSQFTFGPGTKVDIK | |
| 33.3H2VH | Fab-VH | Clostridium | VPI 10463 | TcdA- | QVQLVESGGGVVQPGRSLRLSCAASG | 47 |
| | | difficile | | Transmembrane | FTFNKYGMHWVRQAPGKGLEWVAV | |
| | | | | domain | IWYDGTNKYYADSMKGRFTISRDNS | |
| | | | | | KNMLYLQMNSLRAEDTAVYYCARDP | |
| | | | | | PTANYWGQGTLVTVSS | |
| 33.3H2VL | Fab-VL | Clostridium | VPI 10463 | TcdA-TM domain | DIQMTQSPSSLSASVGDRVTITCRASQ | 48 |
| | | difficile | | | GISSWLAWYQQKPEKAPKSLIYAASS | |
| | | | | | LQSGVPSRFSGSGSGTDFTLTISSLQPE | |
| | | | | | DFATYYCQQYKSYPVTFGGGTKVEIK | |
| Sal4 | Fab | Salmonella | | Salmonella Tm 05 | Source: Richards et al., | 49 |
| | | Tm | | antigen | PLOS Negl Trop | |
| | | | | | Dis 14(3):e0007803, 2020 | |
| PeA3 | Fab | Salmonella | | Salmonella Tm 05 | Source: Richards et al., | 50 |
| | | Tm | | antigen | PLOS Negl Trop | |
| | | | | | Dis 14(3):e0007803, 2020 | |
| AbiSeO7 | sdAb | Salmonella | | Salmonella | QVQLVESGGGLVQAGGSLRLSCTDSG | 51 |
| | | Enterica | | enterica | RTFSVKPMGWFRQAPGMEREFVAAA | |
| | | | | FliC | SFTGVSTFYADSVKDRFTIFRDKDKN | |
| | | | | | TMDLQINSLKPEDTGAYYCAGTTRTL | |
| | | | | | WGSKWRDVLEYEYWGQGTQVTVSS | |
| AR20.5 VL | Fab | | | mucin1 peptide | DVLMTQTPLSLPVSLGDQASISCRSSQ | 52 |
| | | | | | TIVHSNGKIYLEWYLQKPGQSPKLLIY | |
| | | | | | RVSKRFSGVPDRFSGSGSGTDFTLKIS | |
| | | | | | RVEAEDLGVYYCFQGSHVPWTFGGG | |
| | | | | | TKLEIKRADAAPTVSIFPPSSEQLTSGG | |
| | | | | | ASVVCFLNNFYPKDINVKWKIDGSER | |
| | | | | | QNGVLNSWTDQDSKDSTYSMSSTLT | |
| | | | | | LTKDEYERHNSYTCEATHKTSTSPIVK | |
| | | | | | SFNR | |
| AR20.5 VH | Fab | | | mucin1 peptide | EVKLVESGGGLVAPGGSLKLSCAASG | 53 |
| | | | | | FTFSSYPMSWVRQTPEKRLEWVAYIN | |
| | | | | | NGGGNPYYPDTVKGRFTISRDNAKNT | |
| | | | | | LYLQMSSLKSEDTAIYYCIRQYYGFD | |
| | | | | | YWGQGTTLTVSSAKTTPPSVYPLAPG | |
| | | | | | SAAQTNSMVTLGCLVKGYFPEPVTVT | |
| | | | | | WNSGSLSSGVHTFPAVLQSDLYTLSS | |
| | | | | | SVTVPSSTWPSETVTCNVAHPASSTK | |
| | | | | | VDKKIVP | |
| MEDI4893 | Fab | | | S. aureus alpha | Source: Jones-Nelson et al., | 54 |
| | | | | toxin | Antimicrob Agents Chemother | |
| | | | | | 64(5):e02347-19, 2020 | |
| CAA1 | Fab | Campylobacter | | Campylobacter FliD | Source: Perruzza et al., | 55 |
| | | Jejuni | | | Front Immunol 11:1011, 2020 | |
| CCG4 | Fab | Campylobacter | | Campylobacter FliD | Source: Perruzza et al., | 56 |
| | | Jejuni | | | Front Immunol 11:1011, 2020 | |
| PCG4 | Fab-VL | Clostridium | | TcdA | DVVMTQTPLSLPVSLGDQASMSCRSS | 57 |
| | | difficile | | | QSLVHNNGDTYLHWYLQKPGRSPKL | |
| | | | | | LLHKVSNRLSGVPDRFSGSGSGTDFT | |
| | | | | | LKISRVETEDLGVYFCSQSTHVPWTF | |
| | | | | | GGGTKLEIK | |
| A1.3 | sdAb | Clostridium | VPI 10463 | TcdA (amino acid | QVKLEESGGGLVQAGGSLRLSCAASI | 58 |
| | | difficile | | residues 2304-2710) | RSFSYRNMGWFRQPPGKEREFVAAIT | |
| | | | | | WDGGSTRYADSVKGRFTVSRDNAKK | |
| | | | | | TVYLQMNSLKPEDAAVYYCAAGFGH | |
| | | | | | TLATSSDEYDYWWGQGTQVTVSS | |
| A4.2 | sdAb | Clostridium | VPI 10463 | TcdA (amino acid | QVKLEESGGGLVQAGGSLRLSCAASG | 59 |
| | | difficile | | residues 2304-2710) | RTFNTLSMGWFRQAPGKEREFVAAV | |
| | | | | | SRSGGSTYYADSVKGRFTISRDNAKN | |
| | | | | | TVYLQMNSLKPEDTAVYYCAAAATK | |
| | | | | | SNTTAYRLSFDYWGQGTQVTVSS | |
| A19.2 | sdAb | Clostridium | VPI 10463 | TcdA (amino acid | QVKLEESGGGLVQPGGSLRLSCAASG | 60 |
| | | difficile | | residues 2304-2710) | RTLSSYIVAWFRQAPGKEREFVAGISR | |
| | | | | | RGGNSAYVESVKGRFTISRDNAKNTV | |
| | | | | | YLQMNSLKPEDTAVYYCAADGSVAG | |
| | | | | | WGRRSVSVSSYDYWGQGTQVTVSS | |
| A24.1 | sdAb | Clostridium | VPI 10463 | TcdA (amino acid | QVQLVESGGGLVQAGGSLRLSCAASI | 61 |
| | | difficile | | residues 2304-2710) | RSFSNRNMGWFRQPPGKEREFVAGIS | |
| | | | | | WGGGSTRYADSVKGRFTISRDNAKK | |
| | | | | | TVYLQMNSLKPEDTAVYYCAAEFGH | |
| | | | | | NIATSSDEYDYWGQGTQVTVSS | |
| A26.8 | sdAb | Clostridium | VPI 10463 | TcdA (amino acid | QVQLEESGGGLVQAGGSLRLSCAASE | 62 |
| | | difficile | | residues 2304-2710) | RTFSRYPVAWFRQAPGAEREFVAVIS | |
| | | | | | STGTSTYYADSVKGRFTISRDNAKVT | |
| | | | | | VYLQMNNLKREDTAVYFCAVNSQRT | |
| | | | | | RLQDPNEYDYWGQGTQVTVSS | |
| VHH2 | sdAb | Clostridium | Strains | LMW subunit of the | QVKLEESGGGLVQPGGSLRLSCAASG | 63 |
| | | difficile | QCD-32g58 | SLP | GTFTNYAMAWFRQTPGNDREFVGIIS | |
| | | | (GenBank | | QKGGRTYYADSVKGRFSVSRDNAKN | |
| | | | Acc. No. | | TAYLQMNSLKPEDTAVYYCAAGDSS | |
| | | | AAML00000000) | | YYYTRSRYDIWGQGTQVTVSS | |
| VHH12 | sdAb | Clostridium | Strains | LMW subunit of the | QVQLVESGGGLVQAGDSLRLSCAAS | 64 |
| | | difficile | QCD-32g58 | SLP | GGTFSSYAVGWFRQAPGKERQFVAAI | |
| | | | (GenBank | | SWSGRSTEYADSVKGRFTISRDNAKN | |
| | | | Acc. No. | | TVYLQMNSLQPDDTGVYYCAADWS | |
| | | | AAML00000000) | | HPENKAELLRLRLWVLSAESSDYWG | |
| | | | | | QGTQVTVSS | |
| VHH22 | sdAb | Clostridium | Strains | LMW subunit of the | QVQLVESGGGLVQPGGSLRLSCAASG | 65 |
| | | difficile | QCD-32g58 | SLP | FTLDSYAIGWFRQAPGKEHEGISCISS | |
| | | | (GenBank | | NGGSTYYTDSVKGRFTISRDNAKNTV | |
| | | | Acc. No. | | YLQMNSLESEDSAVYYCATVRRCSSL | |
| | | | AAML00000000) | | DMALGALATRTKGYDYWGQGTQVT | |
| | | | | | VSS | |
| VHH23 | sdAb | Clostridium | Strains | LMW subunit of the | QVQLVESGGGLVQAGGSLRLSCAAS | 66 |
| | | difficile | QCD-32g58 | SLP | GRTFSSYAVGWFRQAPGKEREFVAAI | |
| | | | (GenBank | | SWSGGYTDYADSVKGRFTISRDNAK | |
| | | | Acc. No. | | NTVYLQMNNLKPEDTAVYYCAADW | |
| | | | AAML00000000) | | SHPENKAELLRLRLWVLSAESSDDW | |
| | | | | | GQGTQVTVSS | |
| VHH26 | sdAb | Clostridium | Strains | LMW subunit of the | QVKLEESGGGLVQAGGSLRLSCAASG | 67 |
| | | difficile | QCD-32g58 | SLP | RTFTNYAMAWFRQASGKEREFVAIIS | |
| | | | (GenBank | | QSGGRTYYGDSVKGRFTISRDNAKNT | |
| | | | Acc. No. | | VYLQLNSLQPEDTGVYYCAAGDSPY | |
| | | | AAML00000000) | | YSRSRYDLWGPGTQVTVSS | |
| VHH49 | sdAb | Clostridium | Strains | LMW subunit of the | QVKLEESGGGLVQAGGSLRLSCAASE | 68 |
| | | difficile | QCD-32g58 | SLP | GAFSDALSRHAAGWFRQAPGKEREF | |
| | | | (GenBank | | VAAISWNGANTDYKNSVNNRFTISRD | |
| | | | Acc. No. | | TSKNTVYLQMNSLKPEDTAVYYCAA | |
| | | | AAML00000000) | | NEPSWISRIYYRGLRYDLWGQGTQVT | |
| | | | | | VSS | |
| VHH50 | sdAb | Clostridium | Strains | LMW subunit of the | QVKLEESGGGLVQPGGSLRLSCAASG | 69 |
| | | difficile | QCD-32g58 | SLP | FTLDYYTIGWFRQAPGKEREGVACIS | |
| | | | (GenBank | | SDDRTYYVDSVKGRFAISRDNVKNTV | |
| | | | Acc. No. | | YLQMNNLKPEDTAYYCASKESVFLIA | |
| | | | AAML00000000) | | TMKGCAPGHDYYWGQGTQVTVSS | |
| CAMELID | sdAb | HIV-1 | | capsid protein | QVQLVESGGGLVQAGGSLRLSCAAS | 70 |
| VHH 9 | | | | | GSFFMSNVMAWYRQAPGKARELIAA | |
| | | | | | IRGGDMSTVYDDSVKGRFTITRDDDK | |
| | | | | | NILYLQMNDLKPEDTAMYYCKASGS | |
| | | | | | SWGQGTQVTVSSHHHHHH | |
| B5.2 | sdAb | Clostridium | VPI 10463 | TcdB-RBD | QVQLVESGGGLVQPGGSLRLSCAASG | 71 |
| | | difficile | | | NIFSINTMGWYRQAPGKQLELVAAIT | |
| | | | | | SGGTTSYTDSVEGRFTISRDNAKNAV | |
| | | | | | YLQMNSLKAEDTAVYYCNTVKVVG | |
| | | | | | GRLDNPDYWGQGTQVTVSS | |
| B7.3 | sdAb | Clostridium | VPI 10463 | TcdB-RBD | QVKLEESGGGLVQPGGSLRLSCAASG | 72 |
| | | difficile | | | RTASGYGMGWFRQAPGKEREFVAAI | |
| | | | | | SRSGAGTLNADFVKGRFTISRDNAKN | |
| | | | | | TVYLQMNSLKPEDTAVYYCVARPTK | |
| | | | | | VDRDYATRREMYNYWGQGTQVTVSS | |
| B13.2 | sdAb | Clostridium | VPI 10463 | TcdB-RBD | QVKLEESGGGSVQAGGSLRLSCAASG | 73 |
| | | difficile | | | RDFSTLAMGWFRQAPGKEREFVATIN | |
| | | | | | WSGGTTHYADSVKGRFTISRDNAKN | |
| | | | | | TVYLQMGSLKPEDTAVYYCGRSKYA | |
| | | | | | AGALTRAYDYNYWGQGTQVTVSS | |
| B13.3 | sdAb | Clostridium | VPI 10463 | TcdB-RBD | QVKLEESGGGLVQAGGSLRLSCSASG | 74 |
| | | difficile | | | SIFSINDMGWYRRAPGKRRELVAAITS | |
| | | | | | GGIPNYADSVKGGRFTISRDNAKNTG | |
| | | | | | YLQMNSLKPEDTAVYYCAAQFGTVA | |
| | | | | | AALRRHEYDYWGQGTQVTVSS | |
| B13.6 | sdAb | Clostridium | VPI 10463 | TcdB-RBD | QVKLEESGGGLVQAGGSLRLSCSASG | 75 |
| | | difficile | | | RTFSSGVMGWFRQAPGKQRELVAAIT | |
| | | | | | TGGSTSYTDSVKGGRFTISRDNAKNT | |
| | | | | | VYLQMNSLKPEDTAVYYCNSVAVVG | |
| | | | | | GVIKSPDYWGQGTQVTVSS | |
| B15.3 | sdAb | Clostridium | VPI 10463 | TcdB-RBD | QVQLVESGGGSVQAGGSLRLSCAAS | 76 |
| | | difficile | | | GLSRYAMAWFRQGTGKEREFVASTN | |
| | | | | | WSSGNTPYADSVKGGRFIISRDNAKN | |
| | | | | | TVYLQMNSLKPGDTAIYYCAARKLD | |
| | | | | | VPSRYSQHYDYWGQGTQVTVSS | |
| B15.5 | sdAb | Clostridium | VPI 10463 | TcdB-RBD | QVQLVESGGDLVQAGGSLRLSCAAS | 77 |
| | | difficile | | | GSISRISTMGWYRQAPGKQRELVATIS | |
| | | | | | TGGTTNYAESVKGGRFTVSRDNAKN | |
| | | | | | TMYLQMNSLKPEDTAVYYCAAGWK | |
| | | | | | VVRGSLEYEYSGQGTQVTVSS | |
| CAN_356 | sdAb | human | | Human noroviruses | DVQLVESGGGLVQPGGSLRLSCAASG | 78 |
| | | norovirus | | capsid protein | SIFSIYAMGWYRQAPGKQRELVASISS | |
| | | | | | GGGTNYADSVKGRFTISGDNAKNTV | |
| | | | | | YLQMNSLKPEDTAVYYCKREDYSAY | |
| | | | | | APPSGSRGRGTQVTVSSHHHHHH | |
| CAN_388 | sdAb | human | | Human noroviruses | DVQLVESGGGLVQPGGSLRLSCAASG | 79 |
| | | norovirus | | capsid protein | SIFSIYAMGWYRQAPGKQRELVASISS | |
| | | | | | GGGTNYADSVKGRFTISGDNAKNTV | |
| | | | | | YLQMNSLKPEDTAVYYCKREDYSAY | |
| | | | | | APPSGSRGRGTQVTVSSHHHHHH | |
| CAN_389 | sdAb | human | | Human noroviruses | DVQLVESGGGLVQPGGSLRLSCAASE | 80 |
| | | norovirus | | capsid protein | SILSFNHMAWYRQGPGEQRELVAVIT | |
| | | | | | REGSTDYADSVKGRFTISRDNAKNM | |
| | | | | | VYLLMSNLRPEDTAVYYCNRGISNP | |
| | | | | | WGQGTQVTVSSHHHHHH | |
| 7B2 (VL) | Fab | HIV | | virions | METDTLLLWVLLLWVPGSTGDDIQM | 81 |
| | | | | | TQSPASLAVPLLLWISGAYGDIVLAQS | |
| | | | | | PDSLAVSPGERATIHCKSSQTLLYSSN | |
| | | | | | NRHSIAWYQQRPGQPPKLLLYWASM | |
| | | | | | RLSGVPDRFSGSGSGTDFTLTINNLQA | |
| | | | | | EDVAIYYCHQYSSHPPTFGHGTRVEL | |
| | | | | | RRTVAAPSVFIFPPSDEQLKSGTASVV | |
| | | | | | CLLNNFYPREAKVQWKVDNALQSGN | |
| | | | | | SQESVTEQDSKDSTYSLSSTLTLSKAD | |
| | | | | | YEKHKVYACEVTHQGLSSPVTKSFNR | |
| | | | | | GEC | |
| 7B2 (VH) | Fab | HIV | | virions | METDTLLLWVLLLWVPGSTGDQVQL | 82 |
| | | | | | VQSGGGVFKPGGSLRLSCEASGFTFT | |
| | | | | | EYYMTWVRQAPGKGLEWLAYISKNG | |
| | | | | | EYSKYSPSSNGRFTISRDNAKNSVFLQ | |
| | | | | | LDRLSADDTAVYYCARADGLTYFSEL | |
| | | | | | LQYIFDLWGQGARVTVSSASTKGPSV | |
| | | | | | FPLAPSSKSTSGGTAALGCLVKDYFPE | |
| | | | | | PVTVSWNSGALTSGVHTFPAVLQSSG | |
| | | | | | LYSLSSVVTVPSSSLGTQTYICNVNHK | |
| | | | | | PSNTKVDKRVEPKSCDK | |
| SD36 | sdAb | Influenza A | Group 2 (H3, H4, | Hemagglutinin (HA) | EVQLVESGGGLVQAGGSLKLSCAAS | 116 |
| | | | H7, H10) | | GRTYAMGWFRQAPGKEREFVAHINA | |
| | | | | | LGTRTYYSDSVKGRFTISRDNAKNTE | |
| | | | | | YLEMNNLKPEDTAVYYCTAQGQWR | |
| | | | | | AAPVAVAAEYEFWGQGTQVTVS | |
| SD38 | sdAb | Influenza A | Group 1 (H1, H2, | Hemagglutinin (HA) | EVQLVESGGGLVQPGGSLRLSCAVSIS | 117 |
| | | | H5) Group 2 (H3, | | IFDIYAMDWYRQAPGKQRDLVATSFR | |
| | | | H7, H10) | | DGSTNYADSVKGRFTISRDNAKNTLY | |
| | | | | | LQMNSLKPEDTAVYLCHVSLYRDPL | |
| | | | | | GVAGGMGVYWGKGALVTVSS | |
| SD83 | sdAb | Influenza B | | Hemagglutinin (HA) | EVQLVESGGGLVQPGGSLRLSCAATG | 118 |
| | | | | | FTLENKAIGWFRQTPGSEREGVLCISK | |
| | | | | | SGSWTYYTDSMRGRFTISRDNAENTV | |
| | | | | | YLQMDSLKPEDTAVYYCATTTAGGG | |
| | | | | | LCWDGTTFSRLASSWGQGTQVTVSS | |

B. Modifications to Confer Specific Binding by Replacement of the D2 Domain with a Member of a Specific Binding Pair or an Endolysin

In some implementations of the recombinant polypeptides disclosed herein, the D2 domain is modified by substitution with a non-antibody protein or protein domain that confers the ability to bind a target molecule, such as a viral capsid protein, a mucin, a lectin, sialic acid or bacterial peptidoglycan. Table 3 below provides the amino acid sequences of exemplary immunoglobulin domains that can substitute for the D2 domain to confer binding to HIV-1 gp120 or sialic acid. Table 4 provides exemplary proteins, such as members of specific binding pairs, that can be substituted for D2 to confer binding to a variety of different target molecules, including but not limited to, lectin, mucin, biotin, retinol, lactose and other carbohydrates, tRNA, bile acid and integrins. Table 5 provides a list of exemplary endolysins that can be substituted for the D2 domain to confer binding to bacterial peptidoglycan.

TABLE 3
Immunoglobin domains for substitutions of the D2 domain

| | | Description/ | | SEQ |
|---|---|---|---|---|
| Module name | Type | Target | Sequence | ID NO: |
| CD4 (first | Ig- | Cell surface | KKVVLGKKGDTVELTCTASQKKSIQFHWKNSNQIKILGNQGSFLTKG | 92 |
| topological | domain | receptor, binds | PSKLNDRADSRRSLWDQGNFPLIIKNLKIEDSDTYICEVEDQKEEVQL | |
| domain) | | HIV-1 gp120 and | LVFGLTANSDTHLLQGQSLTLTLESPPGSSPSVQCRSPRGKNIQGGKT | |
| | | MHC Class II | LSVSQLELQDSGTWTCTVLQNQKKVEFKIDIVVLAFQKASSIVYKKE | |
| | | molecules | GEQVEFSFPLAFTVEKLTGSGELWWQAERASSSKSWITFDLKNKEVS | |
| | | | VKRVTQDPKLQMGKKLPLHLTLPQALPQYAGSGNLTLALEAKTGKL | |
| | | | HQEVNLVVMRATQLQKNLTCEVWGPTSPKLMLSLKLENKEAKVSKR | |
| | | | EKAVWVLNPEAGMWQCLLSDSGQVLLESNIKVLPTWSTPVQP | |
| Sialic acid- | Ig- | adhesion | KEQKDYLLTMQKSVTVQEGLCVSVLCSFSYPQNGWTASDPVHGYWF | 93 |
| binding Ig-like | domain | molecule, binds | RAGDHVSRNIPVATNNPARAVQEETRDRFHLLGDPQNKDCTLSIRDT | |
| lectin 12 | | sialic acid | RESDAGTYVFCVERGNMKWNYKYDQLSVNVTASQDLLSRYRLEVPE | |
| | | | SVTVQEGLCVSVPCSVLYPHYNWTASSPVYGSWFKEGADIPWDIPVA | |
| | | | TNTPSGKVQEDTHGRFLLLGDPQTNNCSLSIRDARKGDSGKYYFQVE | |
| | | | RGSRKWNYIYDKLSVHVTALTHMPTFSIPGTLESGHPRNLTCSVPWA | |
| | | | CEQGTPPTITWMGASVSSLDPTITRSSMLSLIPQPQDHGTSLTCQVTLP | |
| | | | GAGVTMTRAVRLNISYPPQNLTMTVFQGDGTASTTLRNGSALSVLEG | |
| | | | QSLHLVCAVDSNPPARLSWTWGSLTLSPSQSSNLGVLELPRVHVKDE | |
| | | | GEFTCRAQNPLGSQHISLSLSLQNEYTGKMRPISGVTLGA | |
| Sialic acid- | Ig- | adhesion | FVRTKIDTTENLLNTEVHSSPAQRWSMQVPPEVSAEAGDAAVLPCTF | 94 |
| binding Ig-like | domain | molecule, binds | THPHRHYDGPLTAIWRAGEPYAGPQVFRCAAARGSELCQTALSLHGR | |
| lectin 15 | | sialic acid | FRLLGNPRRNDLSLRVERLALADDRRYFCRVEFAGDVHDRYESRHG | |
| | | | VRLHVTAAPRIVNISVLPSPAHAFRALCTAEGEPPPALAWSGPALGNS | |
| | | | LAAVRSPREGHGHLVTAELPALTHDGRYTCTAANSLGRSEASVYLFR | |
| | | | FHGASGAST | |

TABLE 4
Additional domains for substitution of the D2 domain

| | Source | Antigen/target | | SEQ |
|---|---|---|---|---|
| Module name | species | specificity | Sequence | ID NO: |
| Spr1345 | Streptococcus | Mucin | MGHHHHHHVVPKTATSTETKTITRIIHYVDKVTNQNVKEDVVQP | 95 |
| | pneumoniae | | VTLSRTKTENKVTGVVTYGEWTTGNWDEVISGKIDKYKDPDIPTV | |
| | | | ESQEVTSDSSDKEITVRYDRLST | |
| Streptavidin | Streptomyces | Biotin | MSGSHHHHHHSSGIEGRGRLIKHMTAEAGITGTWYNQLGSTLIVT | 96 |
| | avidinii | | AGADGALTGTYESAVGNAEGSYVLTGRYDSAPATDGSGTALGWT | |
| | | | VAWKNNYRNAHSASTWSGQYVGGAEARINTQVLTTSGTTEANA | |
| | | | WKSTLVGHDTFTKVKPSAASI | |
| Azurin | Pseudomonas | copper | AECSVDIQGNDQMQFNTNAITVDKSCKQFTVNLSHPGNLPKNVM | 97 |
| | aeruginosa | | GHNWVLSTAADMQGVVTDGMASGLDKDYLKPDDSRVIAHTKLI | |
| | | | GSGEKDSVTFDVSKLKEGEQYMFFDTFPGHSALIKGTLTLK | |
| Retinol binding | Homo sapiens | Retinol | MRGSTRDQNGTWEMESNENFEGYMKALDIDFATRKIAVRLTQTK | 98 |
| protein-II | | | VIDQDGDNFKTKTTSTFRNYDVDFTVGVEFDEYTKSLDNRHVKAL | |
| (CRBP_II) | | | VTWEGDVLVCVQKGEKENRGWKQWIEGDKLYLELTCGDQVCRQ | |
| | | | VFKKKLVPR | |
| Galectin-4 | Mus musculus | Lactose and other | MRGSHHHHHHTDPAYVPAPGYQPTYNPTLPYKRPIPGGLSVGMS | 99 |
| | | carbohydrates | VYIQGMAKENMRRFHVNFAVGQDDGADVAFHFNPRFDGWDKV | |
| | | | VFNTMQSGQWGKEEKKKSMPFQKGKHFELVFMVMPEHYKVVV | |
| | | | NGNSFYEYGHRLPVQMVTHLQVDGDLELQSINFLGG | |
| Trbp111 | E. coli | tRNA | METVAYADFARLEMRVGKIVEVKRHENADKLYIVQVDVGQKTL | 100 |
| | | | QTVTSLVPYYSEEELMGKTVVVLCNLQKAKMRGETSECMLLCAE | |
| | | | TDDGSESVLLTPERMMPAGVRVVLDHHHHHH | |
| Bile acid | Danio rerio | Bile acids | MRGSAFNGKWETESQEGYEPFCKLIGIPDDVIAKGRDFKLVTEIVQ | 101 |
| binding | | | NGDDFTWTQYYPNNHVVTNKFIVGKESDMETVGGKKFKGIVSME | |
| protein | | | GGKLTISFPKYQQTTEISGGKLVETSTASGAQGTAVLVRTSKKVLV | |
| | | | PR | |
| Mab58 | Orectolobus | Plasmodium | AWVDQTPRTITKETGESLTIKCVLKDHSCGLSSTTWYRTQLGSTNE | 102 |
| | maculatus | falciparum | KTISIGGRYDETVDKGSKSFSLRISDLRVEDSGTYKCQADYSPSCY | |
| | | | SYPSLESAVEGAGTVLTVK | |
| galectin-8 | Homo sapiens | Lactose and other | GSSGSSGMMLSLNNLQNIIYNPVIPYVGTIPDQLDPGTLIVICGHV | 103 |
| | | carbohydrates | PSDADRFQVDLQNGSSVKPRADVAFHFNPRFKRAGCIVCNTLINEK | |
| | | | WGREEITYDTPFKREKSFEIVIMVLKDKFQVAVNGKHTLLYGHRI | |
| | | | GPEKIDTLGIYGKVNIHSIGFSGPSSG | |
| Bovine beta | Bos taurus | Hydrophobic | LIVTQTMKGLDIQKVAGTWYSLAMAASDISLLDAQSAPLRVYVEE | 104 |
| lactoglobulin | | compounds | LKPTPEGDLEILLQKWENGECAQKKIIAEKTKIPAVFKIDALNENK | |
| | | | VLVLDTDYKKYLLFCMENSAEPEQSLACQCLVRTPEVDDEALEKF | |
| | | | DKALKALPMHIRLSFNPTQLEEQCHI | |
| F17b-G lectin | E. coli | Lectin | VVSFIGSTENDVGPSQGSYSSTHAMDNLPFVYNTGYNIGYQNANV | 105 |
| domain | | (GlcNAc(beta1-2)man) | WRIGGGFCVGLDGKVDLPVVGSLDGQSIYGLTEEVGLLIWMGDT | |
| | | | NYSRGTAMSGNSWENVFSGWCVGNYLSTQGLSVHVRPVILKRNS | |
| | | | SAQYSVQKTSIGSIRMRPYNGSSAGSVQTTVNFSLNPFTLNDT | |
| MucBP domain | Lactobacillus | Mucin | MIEPIKRTQVVTQTIHYRYEDGAVAHDDHVVSLIFTQSGKRDLTN | 106 |
| (fragment 187- | acidophilus | | GKEIWDSKWSLTQTFEALPSPVIIGYTADKPMVGPDEVTVDSKNFL | |
| 294) of the | | | DKQNREETVIYSANTITQNKKDGLEHHHHHH | |
| protein LBA1460 | | | | |
| MucBP domain | Pediococcus | Mucin | THATSTETIHYVNEDGDQVFEDGGGKLDFTRTVTIDDVTNEVVEY | 107 |
| of the adhesion | pentosaceus | | GEWTPVTDDEFAAVTSPDKDGYTPDTSEVAAQKPDMTDGPDGTV | |
| protein PEPE | | | KDVEVTVTYTANPAVATI | |
| Human nectin-3 | Homo sapiens | TcdB | GPIIVEPHVTAVWGKNVSLKCLIEVNETITQISWEKIHGKSSQTVA | 108 |
| full ectodomain | | | VHHPQYGFSVQGEYQGRVLFKNYSLNDATITLHNIGFSDSGKYICK | |
| (D1-D3) | | | AVTFPLGNAQSSTTVTVLVEPTVSLIKGPDSLIDGGNETVAAICIA | |
| | | | ATGKPVAHIDWEGDLGEMESTTTSFPNETATIISQYKLFPTRFARG | |
| | | | RRITCVVKHPALEKDIRYSFILDIQYAPEVSVTGYDGNWFVGRKGV | |
| | | | NLKCNADANPPPFKSVWSRLDGQWPDGLLASDNTLHFVHPLTFN | |
| | | | YSGVYICKVTNSLGQRSDQKVIYISDPPHHHHHH | |
| MAdCAM-1 | Homo sapiens | Integrin α4β7 and a | VKPLQVEPPEPVVAVALGASRQLTCRLACADRGASVQWRGLDTS | 109 |
| | | selectin expressed | LGAVQSDTGRSVLTVRNASLSAAGTRVCVGSCGGRTFQHTVQLL | |
| | | on leukocytes | VYAFPNQLTVSPAALVPGDPEVACTAHKVTPVDPNALSFSLLVGG | |
| | | | QELEGAQALGPEVQEEEEEPQGDEDVLFRVTERWRLPPLGTPVPP | |
| | | | ALYCQATMRLPGLELSHRQAIPVLHSPTSPE | |
| Defensin-5 | Homo sapiens | Gram-positive and | ESLQERADEATTQKQSGEDNQDLAISFAGNGLSALRTSGSQARAT | 110 |
| | | Gram-negative | CYCRTGRCATRESLSGVCEISGRLYRLCCR | |
| | | bacteria | | |
| Defensin 6 | Homo sapiens | Gram-positive and | AFTCHCRRSCYSTEYSYGTCTVMGINHRFCCL | 111 |
| | | Gram-negative | | |
| | | bacteria | | |
| FedF adhesin | E. coli | Lectin | NSSASSAQVTGTLLGTGKTNTTQMPALYTWQHQIYNVNFIPSSSG | 112 |
| | | | TLTCQAGTILVWKNGRETQYALECRVSIHHSSGSINESQWGQQSQ | |
| | | | VGFGTACGNKKCRFTGFEISLRIPPNAQTYPLSSGDLKGSFSLTNK | |
| | | | EVNWSASIYVPAIAK | |
| Lactobacilli | Lactobacillus | mucin | MQTAYVKYVDDTTGETLRQDDLHGYTDETIPYSTAEGIKKYEGD | 113 |
| mub-RV | reuteri | | GYVLVSDGFKPGTKFGVGTPTYEVHFKHGMTHTDATDKNAEQKT | |
| | | | VTETIHYVDENNQTVQPDSTTAVTFKRGYTTDNVTGKVVSYDPW | |
| | | | TVDGNQADSKTFAAVPSPAVEGYTPNHQQINEFTVTPDSKDIVKT | |
| | | | VVYVGDP | |

TABLE 5
Endolysins for substitutions of the D2 domain

| Module name | Type | Description/Target | Reference |
|---|---|---|---|
| CD27L | endolysin | endolysin of bacteriophage | Mayer et al., J Bacteriol 193(19): 5477-5486, 2011 |
| | | CD27 targeting Clostridia | |
| | | difficile | |
| PlyC | endolysin | phage endolysin targeting | Nelson et al., Proc Natl Acad Sci USA 98: 4107-4112, 2001; Nelson et al., J Bacteriol |
| | | S. pyogenes, S. uberis | 185(11): 3325-3332, 2003; Nelson et al., Proc Natl Acad Sci USA 103(28): 10765- |
| | | S. equi | 10770, 2006 |
| PlyGBS | endolysin | endolysin targeting Group B | Cheng et al., Antimicrob Agents Chemother 49(1): 111-117, 2005; Cheng and Fischetti, |
| | | streptococci (all serotypes) | Appl Microbiol Biotechnol 74(6): 1284-1291, 2007 |
| | | S. pyogenes | |
| | | Group D streptococci | |
| | | Group L streptococci | |
| | | S. salivarius | |
| Cpl-1 | endolysin | endolysin targeting | McCullers et al., PLoS Pathogens 3: e28, 2007; Loeffler et al., Science 294: 2170-2172, |
| | | S. pneumoniae | 2001; Loeffler et al., Infect Immun 71: 6199-6204, 2003; Loeffler and Fischetti, |
| | | | Antimicrob Agents Chemother 47: 375-377, 2003; Grandgirard et al., J Infect Dis |
| | | | 197: 1519-1522, 2008 |
| PlyV12 | endolysin | endolysin targeting | Yoong et al., J Bacteriol 186: 4808-4812, 2004 |
| | | E. faecalis (VRE) | |
| | | E. faecium | |
| | | S. pyogenes | |
| | | Groups B, C, E, F, L, N | |
| | | streptococci | |
| | | S. uberis | |
| | | S. gordinii | |
| | | S. intermedius | |
| | | S. parasanguis | |
| | | S. aureus | |
| ClyS | endolysin | endolysin targeting | Daniel et al., Antimicrob Agents Chemother 54: 1603-1612, 2010; Gilmer et al., |
| | | S. aureus (MRSA, VISA) & | Antimicrob Agents Chemother 57: 2743-2750, 2013 |
| | | all other staphylococci | |
| PlyB | endolysin | endolysin targeting | Porter et al., J Mol Biol 366: 540-550, 2007 |
| | | B. anthracis, B. cereus, | |
| | | B. thuringiensis, | |
| | | B. megaterium | |
| PlyG | endolysin | endolysin targeting | Schuch et al., Science 418: 884-889, 2002 |
| | | B. anthracis | |
| PlyPH | endolysin | endolysin targeting | Yoong et al., J Bacteriol 188: 2711-2714, 2006 |
| | | B. anthracis | |

### C. Fluorescent Protein Sequences for Substitution of the D2 Domain

In some implementations of the recombinant polypeptides disclosed herein, the D2 domain is replaced with a fluorescent protein to confer the ability for fluorometric detection. These molecules can be used, for example, to facilitate fluorescent microscopy imaging and/or for determining the location or quantity of cSC-containing molecules (e.g., SIgA or SIgM) in an experiment or diagnostic test. For example, this type of recombinant polypeptide can be used to locate and/or visualize SIgA or SIgM and/or complexes with microbes in a culture, or in mucosal tissue from a patient, animal model or ex vivo experimental system.

Listed below are the amino acid sequences of exemplary fluorescent proteins. Additional fluorescent proteins and their amino acid sequences can be found in publicly accessible databases, such as in FPbase (online at fpbase.org).

mCherry (SEQ ID NO: 83):
MVSKGEEDNMAIIKEFMRFKVHMEGSVNGHEFEIEGEGEGRPYEGTQTA
KLKVTKGGPLPFAWDILSPQFMYGSKAYVKHPADIPDYLKLSFPEGFKW
ERVMNFEDGGVVTVTQDSSLQDGEFIYKVKLRGTNFPSDGPVMQKKTMG
WEASSERMYPEDGALKGEIKQRLKLKDGGHYDAEVKTTYKAKKPVQLPG
AYNVNIKLDITSHNEDYTIVEQYERAEGRHSTGGMDELYK
mRuby (SEQ ID NO: 84):
MNSLIKENMRMKVVLEGSVNGHQFKCTGEGEGNPYMGTQTMRIKVIEGG
PLPFAFDILATSFMYGSRTFIKYPKGIPDFFKQSFPEGFTWERVTRYED
GGVITVMQDTSLEDGCLVYHAQVRGVNFPSNGAVMQKKTKGWEPNTEMM
YPADGGLRGYTHMALKVDGGGHLSCSFVTTYRSKKTVGNIKMPGIHAVD
HRLERLEESDNEMFVVQREHAVAKFAGLGGG
mBanana (SEQ ID NO: 85):
MVSKGEENNMAVIKEFMRFKVRMEGSVNGHEFEIEGEGEGRPYEGTQTA
KLKVTKGGPLPFAWDILSPQFCYGSKAYVKHPTGIPDYFKLSFPEGFKW
ERVMNFEDGGVVTVAQDSSLQDGEFIYKVKLRGTNFPSDGPVMQKKTMG
WEASSERMYPEDGALKGEIKMRLKLKDGGHYSAETKTTYKAKKPVQLPG
AYIAGEKIDITSHNEDYTIVELYERAEGRHSTGGMDELYK
mTangarine (SEQ ID NO: 86):
MASSEDVIKEFMRFKVRMEGSVNGHEFEIEGEGEGRPYEGTQTAKLKVT
KGGPLPFAWDILSPQFCYGSKAYVKHPADIPDYLKLSFPEGFKWERVMN
FEDGGVVTVTQDSSLQDGEFIYKVKLRGTNFPSDGPVMQKKTMGWEASS
ERMYPEDGALKGEIKMRLKLKDGGHYDAEVKTTYMAKKPVQLPGAYKTD
IKLDITSHNEDYTIVELYERAEGRHSTGA
mStrawberry (SEQ ID NO: 87):
MVSKGEENNMAIIKEFMRFKVRMEGSVNGHEFEIEGEGEGRPYEGTQTA
KLKVTKGGPLPFAWDILTPNFTYGSKAYVKHPADIPDYLKLSFPEGFKW
ERVMNFEDGGVVTVTQDSSLQDGEFIYKVKLRGTNFPSDGPVMQKKTMG
WEASSERMYPEDGALKGEIKMRLKLKDGGHYDAEVKTTYKAKKPVQLPG
AYIVGIKLDITSHNEDYTIVELYERAEGRHSTGGMDELYK
mHoneydew (SEQ ID NO: 88):
MASSEDVIKEFMRFKVRMEGSVNGHEFEIEGEGEGRPYEGTQTAKLKVT
KGGPLPFAWDILSPQFMWGSKAYVKHPADIPDYLKLSFPEGFKWERVMN
FEDGGVVTVTQDSSLQDGEFIYKVKLRGTNFPSDGPVMQKKTMGWAATT
ERMYPEDGALKGEIKMRLKLKDGGHYDAEVKTTYMAKKPVQLPGAYKID
GKLDITSHNEDYTIVEQYERAEGRHSTGA
Monomeric ultra-stable GFP (muGFP; SEQ ID
NO: 89):
MSKGEELFTGVVPILVELDGDVNGHKFSVRGEGEGDATNGKLTLKFICT
TGKLPVPWPTLVTTLTYGVLCFSRYPDHMKRHDFFKSAMPEGYVQERTI
SFKDDGTYKTRAEVKFEGDTLVNRIELKGIDFKEDGNILGHKLEYNFNS
HNVYITADKQKNGIKAYFKIRHNVEDGSVQLADHYQQNTPIGDGPVLLP
DNHYLSTQSVLSKDPNEKRDHMVLLEDVTAAGITHGMDELYK
mCardinal (SEQ ID NO: 90):
MVSKGEELIKENMHMKLYMEGTVNNHHFKCTTEGEGKPYEGTQTQRIKV
VEGGPLPFAFDILATCFMYGSKTFINHTQGIPDFFKQSFPEGFTWERVT
TYEDGGVLTVTQDTSLQDGCLIYNVKLRGVNFPSNGPVMQKKTLGWEAT
TETLYPADGGLEGRCDMALKLVGGGHLHCNLKTTYRSKKPAKNLKMPGV
YFVDRRLERIKEADNETYVEQHEVAVARYCDLPSKLGHKLNGMDELYK
Mini singlet oxygen generator (miniSOG; SEQ ID
NO: 91):
MEKSFVITDPRLPDNPIIFASDGFLELTEYSREEILGRNGRFLQGPETD
QATVQKIRDAIRDQREITVQLINYTKSGKKFWNLLHLQPMRDQKGELQY
FIGVQLDG

### V. Exemplary Implementations

Implementation 1. A recombinant polypeptide, comprising a chimeric secretory component (cSC) protein, wherein the D2 domain of the cSC comprises at least one modification that confers specific binding to a target molecule or enables fluorometric or colorimetric detection of the recombinant polypeptide.

Implementation 2. The recombinant polypeptide of implementation 1, wherein the at least one modification of the D2 domain comprises:
substitution of complementarity determining region (CDR)-like loops of the D2 domain with CDRs of a single-domain antibody, a variable heavy (VH) domain or a variable light (VL) domain;
substitution of the D2 domain with a single-domain antibody, a VH domain or a VL domain;
substitution of the D2 domain with a first member of a specific binding pair;
substitution of the D2 domain with an endolysin;
substitution of the D2 domain with a fluorescent protein; or
substitution of the D2 domain with Azurin.

Implementation 3. The recombinant polypeptide of implementation 1 or implementation 2, wherein:
the at least one modification of the D2 domain comprises (i) substitution of CDR-like loops of the D2 domain with CDRs of a single-domain antibody, a VH domain or a VL domain or (ii) substitution of the D2 domain with a single-domain antibody, a VH domain or a VL domain; and
the target molecule is an antigen.

Implementation 4. The recombinant polypeptide of implementation 3, wherein the antigen is a bacterial antigen or a viral antigen.

Implementation 5. The recombinant polypeptide of implementation 4, wherein the bacterial antigen is a Clostridium difficile, Salmonella enterica, Salmonella Tm, Streptococcus pneumoniae, Staphylococcus aureus, Listeria monocytogenes or Campylobacter Jejuni antigen.

Implementation 6. The recombinant polypeptide of implementation 5, wherein the C. difficile antigen comprises the low molecular weight (LMW) subunit of surface layer protein (SLP), flagellin (FliC), lipothechoic acid (LTA3), TcdA or TcdB.

Implementation 7. The recombinant polypeptide of implementation 5, wherein:
the Salmonella enterica antigen comprises FliC;
the Salmonella Tm antigen comprises an O antigen;
the Staphylococcus aureus antigen comprises alpha toxin; or
the Campylobacter Jejuni antigen comprises FliD.

Implementation 8. The recombinant polypeptide of implementation 4, wherein the viral antigen is human immunodeficiency virus (HIV)-1 antigen, a severe acute respiratory syndrome coronavirus 2 (SARS-CoV-2) antigen, an influenza virus antigen or a norovirus antigen.

Implementation 9. The recombinant polypeptide of implementation 8, wherein:
the SARS-CoV-2 antigen comprises a SARS-CoV-2 spike protein;
the HIV-1 antigen comprises an HIV-1 capsid protein or HIV-1 envelope protein;
the influenza virus antigen comprises hemagglutinin (HA) or neuraminidase (NA); or
the norovirus antigen comprises a norovirus capsid antigen.

Implementation 10. The recombinant polypeptide of implementation 1 or implementation 2, wherein:
the at least one modification of the D2 domain comprises substitution of the D2 domain with a first member of a specific binding pair; and
the target molecule is a second member of the specific binding pair.

Implementation 11. The recombinant polypeptide of implementation 10, wherein the first and second members of the specific binding pair respectively comprise:
Spr1345 and mucin;
an angiotensin converting enzyme 2 (ACE2) polypeptide and the SARS-CoV-2 spike protein receptor binding domain;
CD4 and HIV-1 gp120;
streptavidin and biotin;
sialic acid-binding Ig-like lectin 12 (Siglec-12) and sialic acid;
sialic acid-binding Ig-like lectin 15 (Siglec-15) and sialic acid;
azurin and copper;
retinol binding protein-II and retinol;
galectin-4 and lactose;
galectin-8 and lactose; or
trbp111 and tRNA.

Implementation 12. The recombinant polypeptide of implementation 1 or implementation 2, wherein:
the at least one modification of the D2 domain comprises substitution of the D2 domain with an endolysin; and
the target molecule is a bacterial peptidoglycan.

Implementation 13. The recombinant polypeptide of implementation 12, wherein the bacterial peptidoglycan is from Clostridium difficile, Streptococcus pyogenes, Streptococcus uberis, Streptococcus equi, Streptococcus gordinii, Streptococcus intermedius, Streptococcus parasanguis, Streptococcus pneumoniae, Enterococcus faecalis, Enterococcus faecium, Staphylococcus aureus, Bacillus anthracis, Bacillus cereus, Bacillus thuringiensis or Bacillus megaterium.

Implementation 14. The recombinant polypeptide of implementation 1 or implementation 2, wherein the at least one modification of the D2 domain comprises substitution of the D2 domain with a fluorescent protein.

Implementation 15. The recombinant polypeptide of implementation 14, wherein the fluorescent protein comprises mCherry, mRuby, mBanana, mTangerine, mStrawberry, mHoneydew, muGFP, mCardinal or miniSOG.

Implementation 16. The recombinant polypeptide of any one of implementations 1-15, further comprising polymeric IgA or polymeric IgM.

Implementation 17. The recombinant polypeptide of implementation 16, wherein the polymeric IgA is dimeric IgA.

Implementation 18. The recombinant polypeptide of implementation 16 or implementation 17, wherein the polymeric or dimeric IgA specifically binds a mucosal antigen.

Implementation 19. The recombinant polypeptide of implementation 18, wherein the mucosal antigen is a mucin.

Implementation 20. The recombinant polypeptide of any one of implementations 1-19, wherein the amino acid sequence of the polypeptide is at least 90% identical to any one of SEQ ID NOs: 2-29, 114 and 115.

Implementation 21. The recombinant polypeptide of any one of implementations 1-20, wherein the amino acid sequence of the polypeptide comprises any one of SEQ ID NOs: 2-29, 114 and 115.

Implementation 22. A method of treating or inhibiting a Clostridium difficile, Salmonella enterica, Salmonella Tm, Streptococcus pneumoniae, Staphylococcus aureus or Campylobacter Jejuni infection in a subject, comprising administering to the subject a therapeutically or prophylactically effective amount of the recombinant polypeptide of any one of implementations 5-7 and 10-13, thereby treating or inhibiting the infection.

Implementation 23. A method of treating or inhibiting an HIV-1, SARS-CoV-2, influenza virus or norovirus infection in a subject, comprising administering to the subject a therapeutically or prophylactically effective amount of the recombinant polypeptide of any one of implementations 8-11, thereby treating or inhibiting the infection.

Implementation 24. The method of implementation 22 or implementation 23, wherein the recombinant polypeptide is administered orally, intranasally or as a suppository.

The following examples are provided to illustrate certain particular features and/or implementations. These examples should not be construed to limit the disclosure to the particular features or implementations described.

### EXAMPLES

The pIgR plays an important role in delivering SIgA to mucosal secretions, yet functionally why its ectodomain (secretory component—SC) remains attached to SIgA is less clear. SIgA structures reveal that SC is solvent accessible, making it an attractive target for engineering unique binding specificity into SC and SIgA. Accordingly, the examples below describe development of chimeric SC (cSC) and SIgA that can bind noncognate ligands. In particular, these examples describe engineering of chimeric cSC that binds to the opportunistic mucosal pathogen C. difficile, which is known to interact with SIgA in the human gut (Olson et al., J Trauma Acute Care Surg, 2013. 74(4):983-89), as well as to influenza virus HA. Further described is a chimeric sSC in which D2 is replaced with a fluorescent protein (mCherry).

### Example 1: Identification of cSC and cSC-Containing SIgA that Bind Target Epitopes

Mammalian SC comprises five Ig-like domains connected by flexible linkers. In unliganded and SIgA structures, the D2 domain of SC occupies solvent accessible positions; in SIgA, D2 lies at the periphery of the complex, fails to form any direct contacts with dIgA, and is not required for dIgA binding (FIG. 3) (Kumar Bharathkar et al., Elife, 2020. 9:e56098; Stadtmueller et al., J Immunol, 2016. 197(4):1408-14). These characteristics make D2 suitable for functionalization.

### Methods

To generate cSC capable of binding target epitopes, a library of cSC expression constructs was designed. In the library, the D2 domain of each cSC was substituted with a unique binding module having the ability to bind host proteins or antigens, including those produced by C. difficile. Three primary approaches were used: (1) substitution of the entire D2 domain with a single domain antibody fragment (sdAb) against C. difficile antigens (such as a sdAb described in Hussack and Tanha, Clin Exp Gastroenterol, 2016. 9:209-24); (2) substitution of D2 CDR-like loops with CDRs from a single domain antibody fragment (sdAb); and substitution of the D2 domain with non-antibody protein domains (FIG. 3). Binding modules were selected based on two criteria: (1) the ability to interact with a pathogen or toxin (antigen); or (2) the ability to bind a host factor that is unique to or enriched in the mucosa, such as mucins (see Tables 1-5). The cSC and counterpart cSFcα (a SIgA lacking Fabs) or cSIgA were produced and subjected to biochemical analysis and binding assays in order to determine the ability of the chimeric molecules to bind target epitopes.

Strategy 1 includes substituting the D2 domain with a sdAb. The sdAbs are single Ig-variable domains with antigen binding specificity that have been commercially developed from heavy chain-only antibodies found in camelids and sharks, and have been used as a scaffold for biological and therapeutic reagents, such as nanobodies. sdAbs are structurally similar to the SC D2 domain. Strategy 2 is to use the SC D2 domain as a scaffold on which to graft CDRs from antibodies. Grafting CDRs from one Ig variable domain to another has been previously described and when applied to SC D2, it is expected to preserve the structural, biochemical and functional properties of the rest of the SC D2 domain (Stadtmueller et al., Elife, 2016. 5:e10640). Strategy 3 is to substitute D2 with protein domains other than canonical antibody domains, and thereby broaden the target epitopes and types of interaction that chimeric SC can mediate.

Five C. difficile antigens and toxins were selected as targets for cSC (strategies 1 and 2: CDI surface layer proteins (SLPs), flagella (FLiC), lipothechoic acid (LTA3) and toxins TcdA and TcdB (FIG. 3). sdAb have been reported for each of these antigens (Hussack and Tanha, Clin Exp Gastroenterol, 2016. 9:209-24). In addition, human gut pathogen Salmonella enterica antigen FliC was selected as an additional target. Surface antigens and flagella are associated with growth and motility whereas toxins are associated with host-cell damage. Finally, human respiratory pathogen influenza antigen hemagglutinin (HA) was selected as a viral target. Neutralization of these antigens is expected to reduce disease virulence. Following construct design, affinity-tagged cSC is transfected alone or is co-transfected with Fcα and JC to produce cSC and cSFcα (a dimeric Fcα with cSC bound) and resulting proteins and complexes are purified from transiently transfected human cell culture using previously described methods (Kumar Bharathkar et al., Elife, 2020. 9:e56098). Expression constructs encoding individual C. difficile toxin fragment, antigens, and sdAb controls are expressed and purified from transiently transfected human cell culture or from transformed E. coli using previously described methods (Murase et al., J Biol Chem, 2014. 289(4):2331-43; Orth et al., J Biol Chem, 2014. 289(26):18008-21; Kroh et al., J Biol Chem, 2018. 293(3):941-952; Calabi et al., Infect Immun, 2002. 70(10):5770-8; Ghose et al., Emerg Microbes Infect, 2016. 5:e8; Cox et al., Glycoconj J, 2013. 30(9):843-55). To determine if cSC can bind its target ligand, monodisperse cSC and cSFcα are combined with purified ligand and subjected to analytical SEC and/or are used in SPR binding assays, which quantify the binding affinity and/or kinetics of interactions with ligand. Values obtained from SPR are compared to those obtained from published data and/or analogous control experiments, in which binding of a sdAb to the ligand is determined.

For strategy 3, binding modules include human receptor angiotensin convertase enzyme 2 (ACE2) and human receptor CD4, which were chosen to identify cSC with potential to neutralize entry of SARS-Cov-2 and HIV-1, respectively. An additional strategy 3 binding module includes the mucin-binding domain from Spr1345, expressed by the pathogen Streptococcus pneumoniae (pdb code 3NZ3). MucBD was chosen to localize cSC to human mucins and/or to neutralize Streptococcus pneumoniae binding to mucins. It is expected that results from testing this sampling of binding modules will direct the selection of additional targets.

### Results

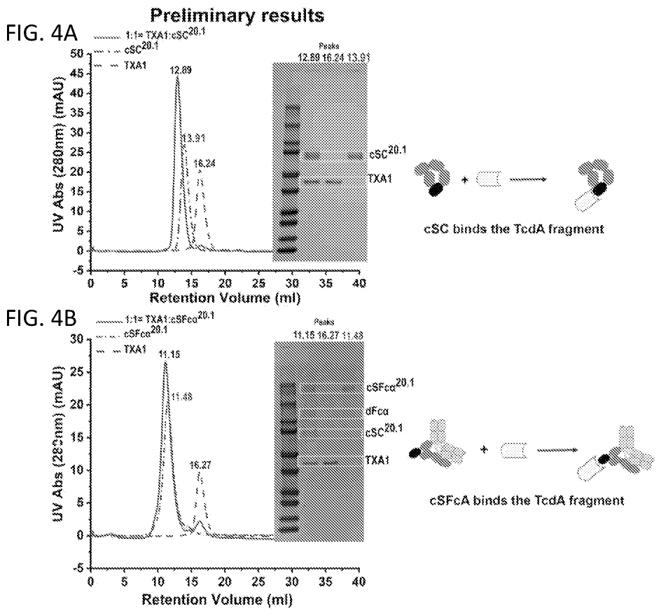

Monodisperse cSC^{20.1} and cSFcα^{20.1}, which encode the sdAb 20.1 (Hussack et al., J Biol Chem, 2011. 286(11):8961-76) (Table 2) in place of SC D2, and its ligand, TcdA fragment TXA1, were produced. Analytical SEC revealed that cSC^{20.1} and cSFcα^{20.1} form complexes with TXA1 (FIG. 4), indicating that a cSC is capable of binding ligand in both its unliganded SC and liganded SFcα conformations. Binding of cSC^{20.1} and cSFcα^{20.11} with TXA1 is quantified using SPR.

Additional studies are performed to test the production and ligand binding capacity of other proposed cSC, including those with grafted CDRs. It is expected that experiments will identify cSC and cSFcα that have the ability to bind C. difficile antigens and toxins through interactions that are not known to occur naturally. Subsequent experiments are performed to test the ability of cSC to neutralize antigens and toxins and to develop bispecific cSIgA that combine cSC with IgA heavy chain and light chain Fabs that also bind a C. difficile antigen. It is also expected that cSC will bind pathogen without compromising Fab functions or blocking interactions with FcR.

### Example 2: Design and Characterization of cSC, cFcα and cSIgA Capable of Neutralizing C. Difficile Toxins and Growth

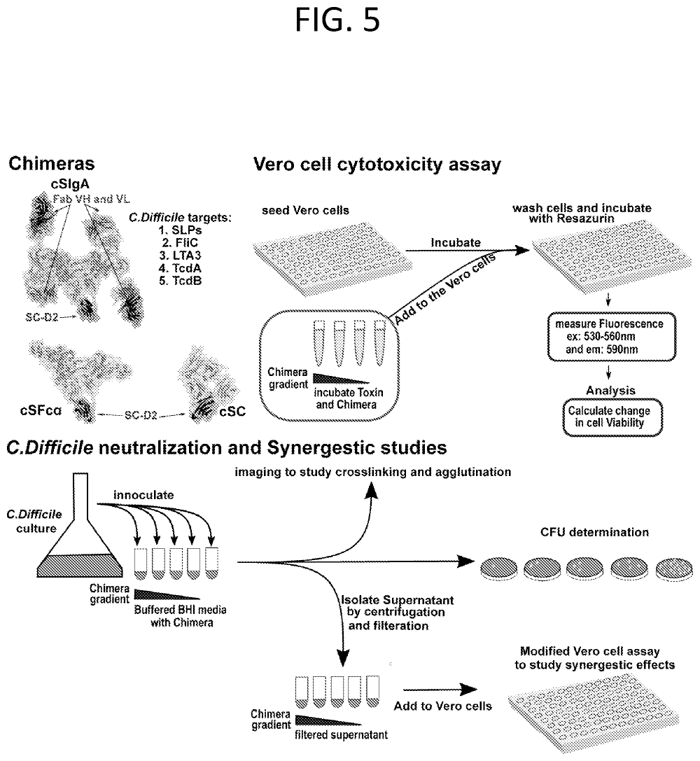

This example describes studies to assay the functional potential of cSC-containing reagents identified in Example 1 and to test their synergy with Fabs that also bind C. difficile antigens. These studies are performed to determine the neutralization potency of cSC, cSFcα and cSIgA variants against C. difficile toxins and growth (FIG. 5). The cSIgA are bispecific, with cSC and the SIgA Fabs both recognizing a unique C. difficile antigen. This design is based on the observation that pathogenic effects of C. difficile are contributed both by secreted C. difficile toxins and by persistent C. difficile growth involving diverse antigens (Yang et al., J Infect Dis, 2014. 210(6):964-72; Kink and Williams, Infect Immun, 1998. 66(5):2018-25; Davies et al., Clin Vaccine Immunol, 2013. 20(3):377-90). The results demonstrated the therapeutic potential of cSC and cSIgA, such as for the treatment of C. difficile infection.

### Methods

To produce cSIgA constructs, expression constructs that fuse anti-C. difficile heavy chain and light chain variable domains with the human IgA heavy chain and light chain constant regions were designed to create IgA with Fabs that target C. difficile antigens (FIG. 5). These constructs were transiently co-transfected with cSC and JC and the resulting cSIgA were purified according to published protocols (Kumar Bharathkar et al., Elife, 2020. 9:e56098). The binding of the cSIgA Fabs were tested against their respective targets by enzyme linked immune sorbent assay (ELISA) and binding of the cSC module in the cSIgA was verified as described in Example 1. The cSC-containing molecules, including cSC^{20.1} and cSFcα^{20.1} were produced as described in Example 1.

The ability of cSCs, cSFcα and cSIgA variants to neutralize toxins TcdA and TcdB was tested using a Vero cell cytotoxicity assay (Anosova et al., Clin Vaccine Immunol, 2015. 22(7):711-25) (FIG. 5). Briefly, a monolayer of Vero cells was infected with toxins at 50% of maximum cytopathic concentration (MC₅₀), in the presence or absence of cSC-containing molecules and the Vero cell viability was determined using Resazurin dye and a standard plate reader (Anosova et al., Clin Vaccine Immunol 22(7):711-725, 2015). In complimentary assays, the ability of cSC-containing molecules to neutralization C. difficile growth was determined by administering variable concentrations of cSCs, cSFcα and cSIgA to a growing culture of C. difficile and subsequently measuring the number of colony forming units (CFU) at defined timepoints following the addition of chimeric molecules (Xie et al., Clin Vaccine Immunol 20(4):517-525, 2013). To evaluate potential synergistic effects of bispecific cSIgA, modified Vero cell assays were conducted in which purified toxins were substituted with supernatants from C. difficile cultures generated for growth neutralization assays (Yucesoy et al., Clin Microbiol Infect 8(7):413-418, 2022) (FIG. 5). In this case, the assay measured the degree to which the chimeric reagent limited and neutralized the total amount of toxin produced during cultured time. A comparison of cSCs, cSFcα and cSIgA against constituent sdAbs was performed along with control experiments utilizing SC, SFcα, SIgA and monomeric IgA, in which SC does not bind C. difficile and Fabs either bind a target C. difficile antigen (positive control) or bind a non-C. difficile antigen (negative control).

### Results

Results described in Example 1 indicated that cSC^{20.1} and cSFcα^{20.1} bind C. difficile TcdA in vitro. Thus, studies were conducted to test whether cSC, cSFcα and cSIgA can neutralize the TcdA and TcdB toxins. Neutralization of C. difficile growth by any reagent is indicated by reduced CFU values compared to controls. Growth reduction correlates with reduced toxin concentration in the media; however, modified Vero cell assays using supernatants from C. difficile cultures are expected to demonstrate whether a single, bi-specific cSIgA can effectively neutralize growth and toxins in a single experimental system. Whereas toxin neutralization occurs when toxins are blocked from entering cells, a decline in C. difficile growth may result from a variety of mechanisms, which are explored using classical agglutination assays and/or motility assays (Kandalaft et al., Appl Microbiol Biotechnol 99(20):8549-8562, 2015).

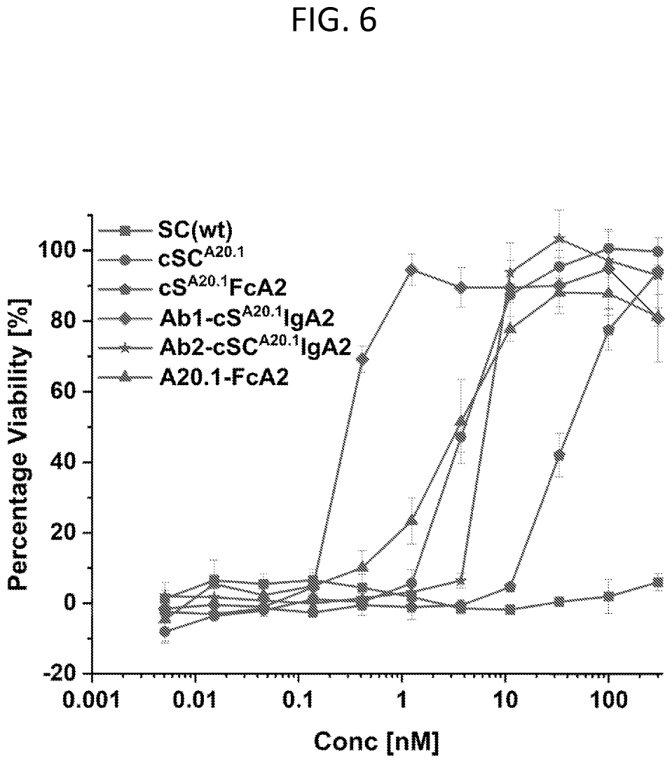

Vero cell assays reporting viability above 50% indicate positive neutralization of toxin by cSC, cSFcα, and/or cSIgA, and when analyzed over a concentration series, can provide an IC50 value for each reagent. Neutralization potency of purified monospecific cSC^{20.1}, cSFcα^{20.1}, and cSIgA, which encode the sdAb 20.1 (Hussack et al., J Biol Chem 286(11):8961-8976, 2011) (Table 2) in place of SC D2, were assayed in Vero cell cytotoxicity assays containing 50 pM TcdA, which causes ˜100% Vero-cell death in normal media. Neutralization curves demonstrated that cSC^{A20.1}, and cFcα and cSIgA variants in which cSC^{A20.1} are in complex with dimeric Fcα (FcA) or dimeric IgAs, neutralize the cytotoxic effects of C. difficile toxin TcdA compared to the wild type SC negative control (FIG. 6).

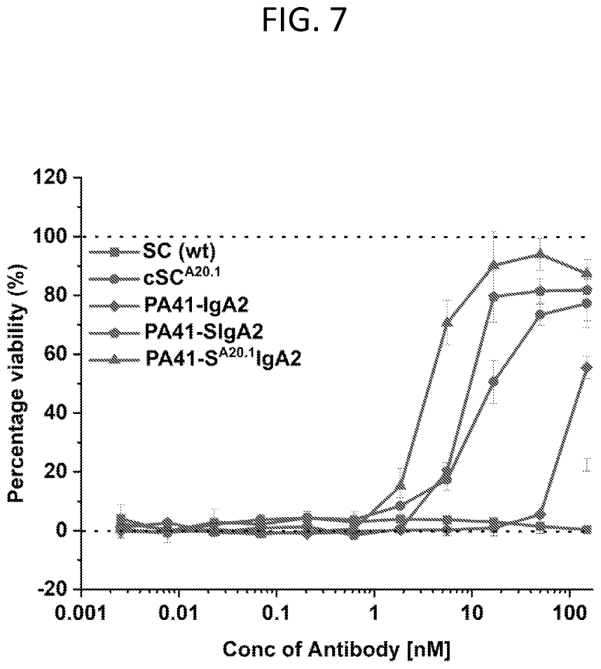

Neutralization potency of bispecific cSIgA was tested in Vero cell cytotoxicity assays containing 50 pM TcdA. Neutralization curves revealed that the bispecific cSIgA PA41-S^{A20.1} IgA2, which incorporates cSC^{A20.1} and antibody PA41 (Kroh et al., J Biol Chem 293(3):941-952, 2018), has enhanced TcdA neutralization potency compared to proteins and complexes that incorporate cSC^{A20.1} or PA41 alone (FIG. 7). The cSIgA (PA41-S^{A20.1}IgA2) is capable of binding different epitopes of TcdA with Fabs (PA41) and cSC (sdAb-A20.1). These results indicate a synergistic effect when cSC and dIgA are used to combine two different antigen binding specificities into a bispecific cSIgA.

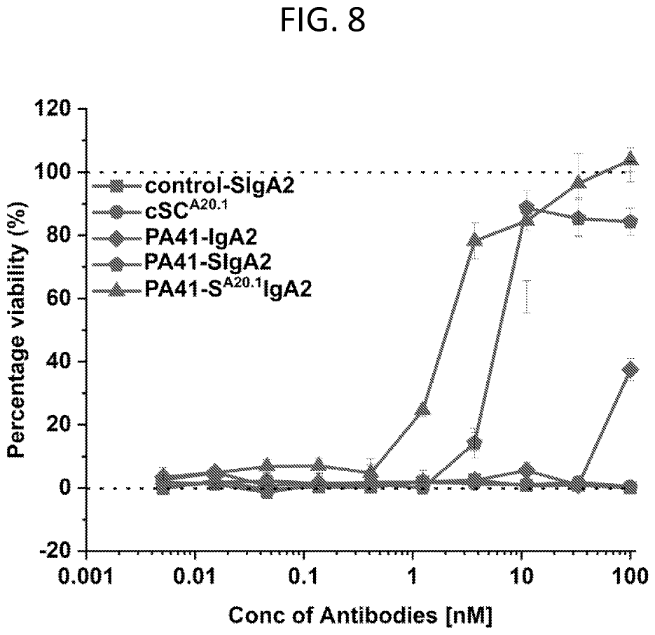

Neutralization potency of bispecific cSIgA was also tested in Vero cell cytotoxicity assays containing 50 pM TcdA and 4 pM TcdB. Fifty pM TcdA and 4 pM TcdB kill ˜100% of Vero cells in normal culture media. Neutralization curves revealed that the addition of proteins containing the PA41 Fab neutralized both TcdA and TcdB, while cSC^{A20.1} neutralized TcdA only. The bispecific cSIgA PA41-S^{A20.1}IgA2, which incorporates cSC^{A20.1} and antibody PA41, showed enhanced neutralization of TcdA and TcdB compared to proteins and complexes that incorporate cSC^{A20.1} or PA41 alone (FIG. 8). These results indicate a synergistic effect on two antigens (TcdA and TcdB) when cSC and dIgA are used to combine two different antigen binding specificities into a bispecific cSIgA.

### Example 3: Design and Characterization of cSC and cSIgA Capable of Facilitating Fluorescence Visualization of C. difficile Antigen

This example describes studies to assay the functional potential of cSC-containing reagents to incorporate a fluorescent protein that links a fluorescence signal to antigen binding, and where relevant, to antigen neutralization. These studies were performed to demonstrate that cSC^{mCherry} can be stably expressed alone and in complex with dIgA (cSIgA). The cSC^{mCherry} replaces the SC D2 domain with the monomeric fluorescent protein mCherry (Shaner et al., Nat Biotechnol 22(12):1567-1572, 2004). cSIgA are bifunctional, with cSC^{mCherry} providing fluorescence and the SIgA Fabs recognizing a C. difficile antigen. The results discussed below demonstrate that cSC and cSIgA can be used to visualize the locations of C. difficile antigens, and ultimately, to uncover mechanisms of neutralization and provide maps of disease progression.

### Methods

The cSC^{mCherry} was designed to replace the SC D2 domain with the monomeric fluorescent protein mCherry (Shaner et al., Nat Biotechnol 22(12):1567-1572, 2004). The cSC^{mCherry} was produced alone and in complex with a dimeric IgA CD5SLP, which is the sdAb-CD5SLP fused to the IgA-Fc (CD5SLP-cS^{mCherry}FcA2). Proteins were produced in transiently transfected mammalian cell culture and were purified from cell supernatant using Ni-NTA resin or Capture Select IgA resin followed by size exclusion elution chromatography (SEC) to evaluate monodispersity and purity. To assay the presence of mCherry signal, fluorescence and the absorbance spectra were measured using 1 μM cSC^{mCherry} in Tris-buffered saline. The absorbance was measured over a range of 500 nm to 800 nm and the fluorescence was measured at 586 nm excitation from 600 nm to 700 nm. To visualize CD5SLP-cS^{mCherry}FcA2 binding to antigen, C. difficile surface layer protein (SLP) was produced and attached to NHS-activated agarose beads using amine coupling. Control agarose beads were prepared by following the same protocol, in the absence of SLP. To assay CD5SLP-cS^{mCherry}FcA2 binding to SLP-coated beads and correlated mCherry signal, the two components were mixed and incubated at room temperature for 1 hour, washed and subjected to brightfield and fluorescence imaging.

### Results

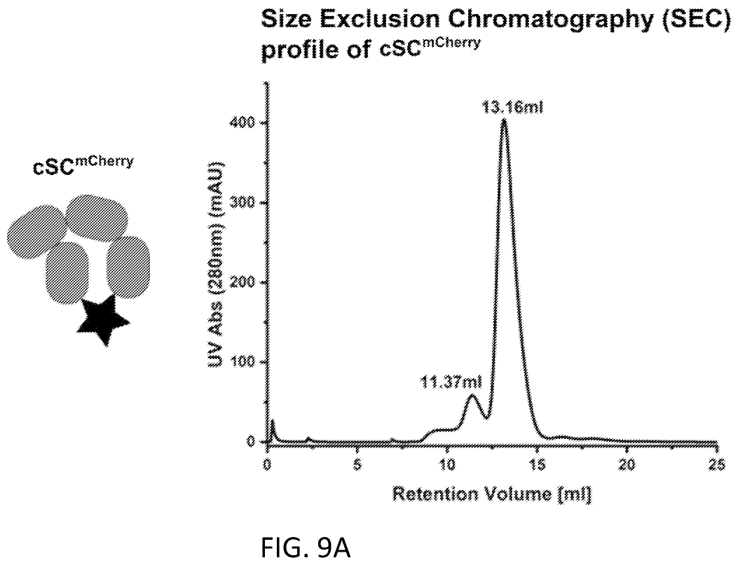

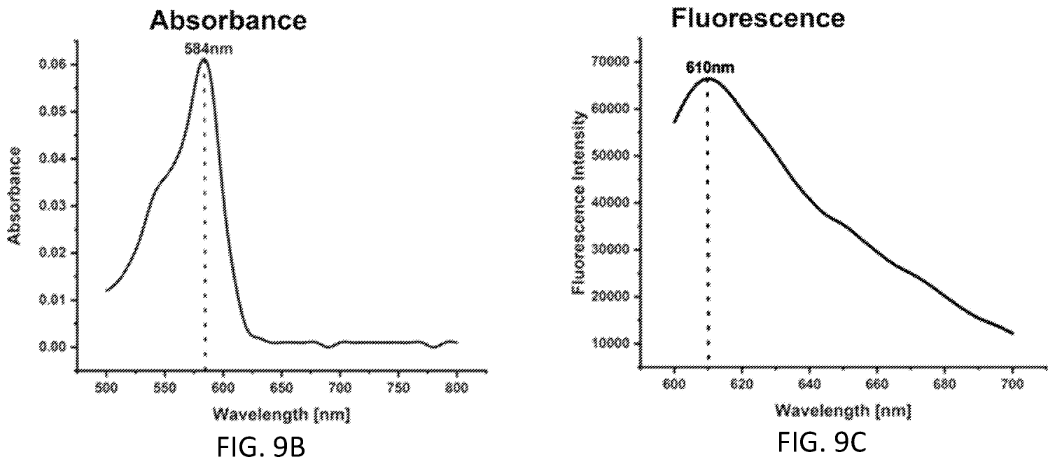

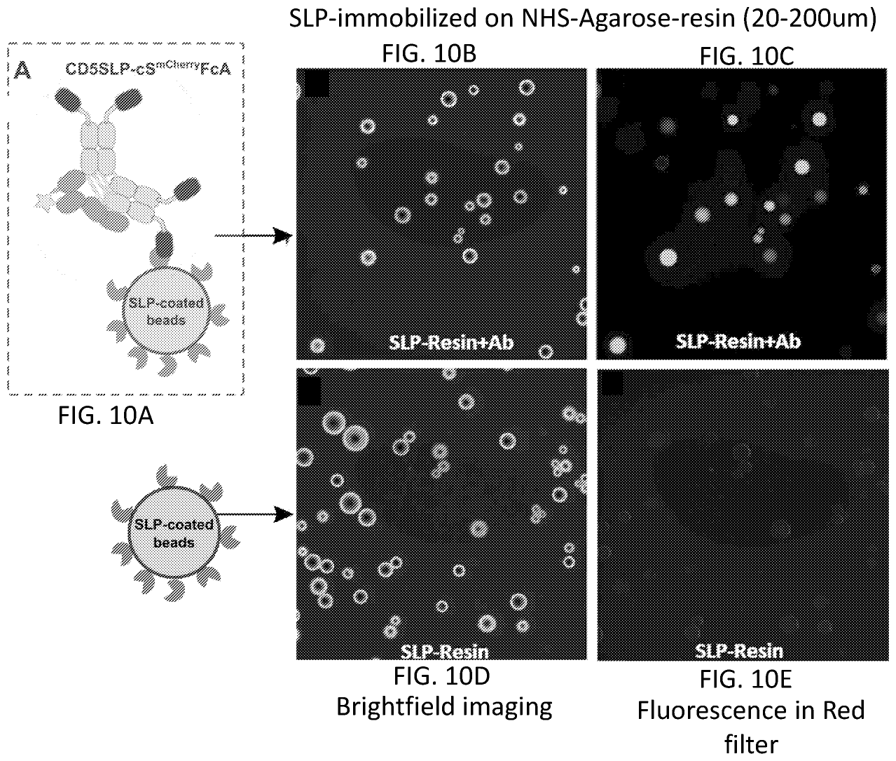

Data indicate that purified cSC^{mCherry}, which has D2 substituted by mCherry, is a monodisperse protein as assayed by SEC (FIG. 9A). Additionally, purified cSC^{mCherry} protein exhibited an expected absorption profile of from 500 to 800 nm, resulting in absorption maxima at 584 nm (FIG. 9B) and expected fluorescence profile from 600 to 700 nm, upon excitation with 586 nm light (FIG. 9C). Fluorescence images revealed red SLP-coupled agarose resin beads, consistent with cSFcA (CD5SLP-S^{mCherry}FcA) binding to SLP-coupled agarose resin beads through the sdAb-CD5SLP, while the bound cSC^{mCherry} confers the fluorescence, allowing the location of the antigen to be imaged (FIGS. 10B-10E). Taken together, these data indicate that SC D2 may be substituted with any monomeric fluorescent protein, which when co-expressed with dIgA recognizing any antigen facilitates visualization and quantification of antigen in an animal model or patient sample. This example also illustrates the potential of SC D2 to be substituted with a protein that does not adopt an Ig fold.

### Example 4: Design and Characterization of cSC that Neutralize Influenza Virus

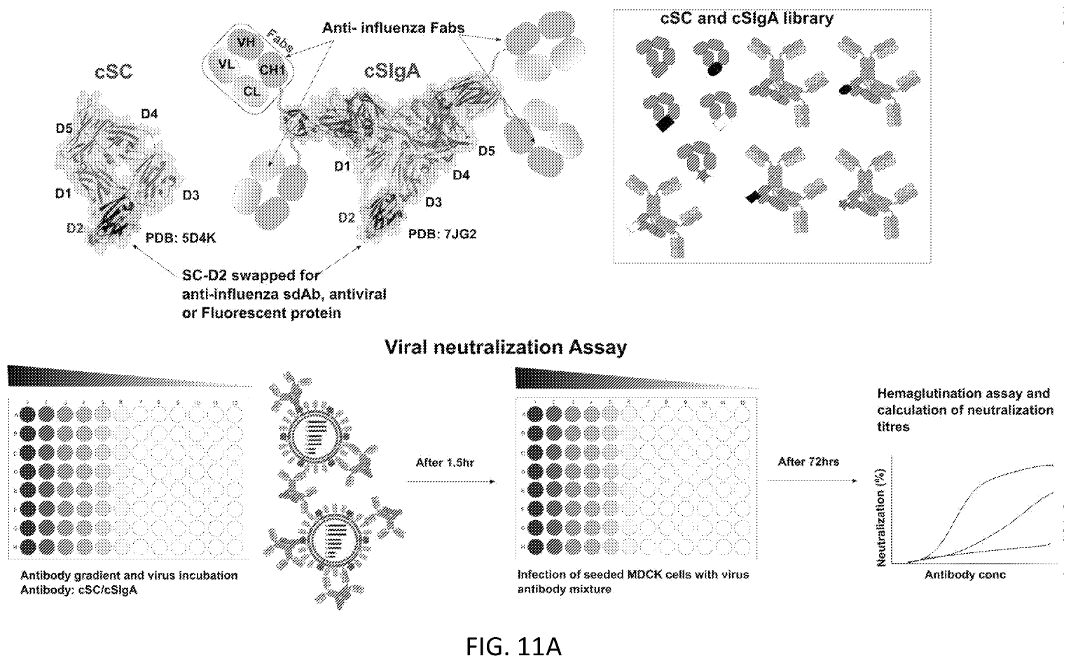

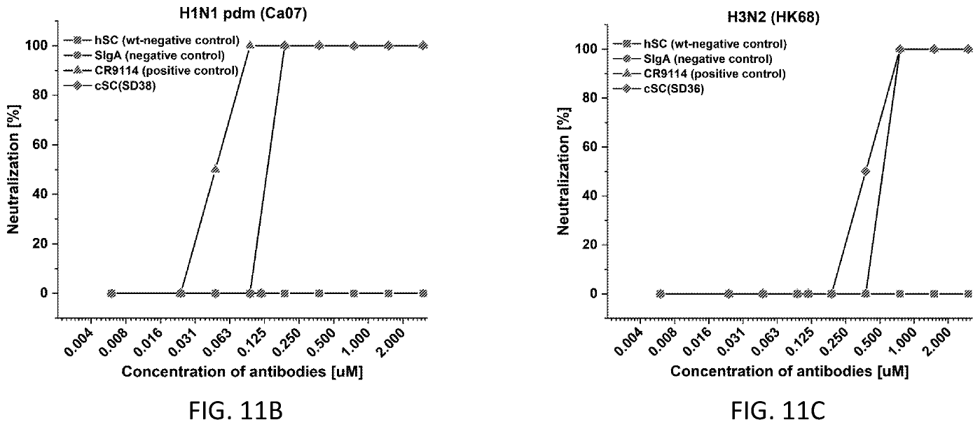

This example describes studies to assay the functional potential of cSC and cSIgA to neutralize a viral antigen. In this example, the viral antigen is influenza virus hemagglutinin (HA). These studies were performed to determine the neutralization potency of cSC variants against influenza in cell-culture based assays (FIG. 11). In these cSC, the entire D2 domain was substituted with a single domain antibody fragment (sdAb) against influenza HA (such as a sdAb described in Laursen et al., Science 362(6414):598-602, 2018). This design is based on the observation that neutralization of viral host-cell entry can prevent or limit the pathological effect of influenza infection. The results provided below demonstrated the therapeutic potential of cSC and cSIgA, such as for the treatment of influenza infection. Additional studies were performed to test the neutralization potency of bispecific cSIgA that combine cSC with IgA heavy chain and light chain Fabs that also bind an influenza antigen.

### Methods

Chimeric SC targeting influenza type A were designed to replace the SC D2 domain with sdAbs SD36 or SD38 to create cSC^{SD36} and cSC^{SD38}. SD36 neutralizes group-2 influenza A virus (H3, H4, H7 and H10), while SD38 neutralizes mainly group-1 influenza A (H1, H2 and H5) (Laursen et al., Science 362(6414):598-602, 2018). cSC^{SD36} and cSC^{SD38} were expressed in transiently transfected mammalian cell culture and purified using Ni-NTA affinity chromatography and SEC. Purified proteins were exchanged into phosphate buffered saline (PBS) and subjected to standard virus neutralization assays (Steel et al., J Virol 83(4):1742-1753, 2009). Briefly, 2-fold dilutions of cSC^{SD36}, cSC^{SD38}, hSC (negative control), and antibody CR9114 (positive control), were mixed with 100 TCID₅₀ of virus, either H1N1 pdm (Ca07) or H3N2 (HK68) and transferred to MDCK monolayers cultured in 96-well flat-bottom plates. Following a 72-hour incubation, virus and antibody-containing media was removed. Subsequently, cell culture was assayed for the presence of HA, which is a measure of whether cells were infected during the 72-hour incubation and if the antibody neutralized infection (FIG. 11A).

### Results

Viral neutralization assays revealed cSC^{SD38} dependent neutralization of H1N1 and cSC^{SD36} dependent neutralization of H3N2. The positive control antibody CR9114, which is a broadly neutralizing antibody capable of neutralizing influenza A and B and its subgroups, showed neutralization while wild type hSC (negative control) did not (FIGS. 11B, 11C). Viral neutralization was concentration-dependent and indicated that cSC incorporating any sdAb recognizing HA can neutralize influenza virus infection.

Additional studies are performed to test the production and neutralization potency of cSC targeting other HA and NA epitopes, as well as cSIgA that combine a cSC (e.g. cSC^{SD38}) with dIgA having Fabs that bind influenza antigens. Based on results from Example 2, it is expected that these experiments will identify additional cSC that can neutralize virus and cSIgA that exhibits enhanced neutralization potency from combining cSC with the dIgA (JC, IgA heavy chain and light chain) having Fabs that also target influenza antigens. It is also expected that cSC will bind pathogen without compromising Fab functions or blocking interactions with FcR.

In view of the many possible implementations to which the principles of the disclosed subject matter may be applied, it should be recognized that the illustrated implementations are only examples of the disclosure and should not be taken as limiting the scope of the disclosure. Rather, the scope of the disclosure is defined by the following claims. We therefore claim all that comes within the scope and spirit of these claims.

1. A recombinant polypeptide, comprising a chimeric secretory component (cSC) protein, wherein the D2 domain of the cSC comprises at least one modification that confers specific binding to a target molecule or enables fluorometric or colorimetric detection of the recombinant polypeptide.

2. The recombinant polypeptide of claim 1, wherein the at least one modification of the D2 domain comprises:
substitution of complementarity determining region (CDR)-like loops of the D2 domain with CDRs of a single-domain antibody, a variable heavy (VH) domain or a variable light (VL) domain;
substitution of the D2 domain with a single-domain antibody, a VH domain or a VL domain;
substitution of the D2 domain with a first member of a specific binding pair;
substitution of the D2 domain with an endolysin;
substitution of the D2 domain with a fluorescent protein; or
substitution of the D2 domain with Azurin.

3. The recombinant polypeptide of claim 1, wherein:
the at least one modification of the D2 domain comprises (i) substitution of CDR-like loops of the D2 domain with CDRs of a single-domain antibody, a VH domain or a VL domain or (ii) substitution of the D2 domain with a single-domain antibody, a VH domain or a VL domain; and
the target molecule is an antigen.

4. The recombinant polypeptide of claim 3, wherein the antigen is a bacterial antigen or a viral antigen.

5. The recombinant polypeptide of claim 4, wherein the bacterial antigen is a Clostridium difficile, Salmonella enterica, Salmonella Tm, Streptococcus pneumoniae, Staphylococcus aureus, Listeria monocytogenes or Campylobacter Jejuni antigen.

6. The recombinant polypeptide of claim 5, wherein:
the C. difficile antigen comprises the low molecular weight (LMW) subunit of surface layer protein (SLP), flagellin (FliC), lipothechoic acid (LTA3), TcdA or TcdB;
the Salmonella enterica antigen comprises FliC;
the Salmonella Tm antigen comprises an O antigen;
the Staphylococcus aureus antigen comprises alpha toxin; or
the Campylobacter Jejuni antigen comprises FliD.

7. (canceled)

8. The recombinant polypeptide of claim 4, wherein the viral antigen is human immunodeficiency virus (HIV)-1 antigen, a severe acute respiratory syndrome coronavirus 2 (SARS-CoV-2) antigen, an influenza virus antigen or a norovirus antigen.

9. The recombinant polypeptide of claim 8, wherein:
the SARS-CoV-2 antigen comprises a SARS-CoV-2 spike protein;
the HIV-1 antigen comprises an HIV-1 capsid protein or HIV-1 envelope protein;
the influenza virus antigen comprises hemagglutinin (HA) or neuraminidase (NA); or
the norovirus antigen comprises a norovirus capsid antigen.

10. The recombinant polypeptide of claim 1, wherein:
the at least one modification of the D2 domain comprises substitution of the D2 domain with a first member of a specific binding pair; and
the target molecule is a second member of the specific binding pair.

11. The recombinant polypeptide of claim 10, wherein the first and second members of the specific binding pair respectively comprise:
Spr1345 and mucin;
an angiotensin converting enzyme 2 (ACE2) polypeptide and the SARS-CoV-2 spike protein receptor binding domain;
CD4 and HIV-1 gp120;
streptavidin and biotin;
sialic acid-binding Ig-like lectin 12 (Siglec-12) and sialic acid;
sialic acid-binding Ig-like lectin 15 (Siglec-15) and sialic acid;
azurin and copper;
retinol binding protein-II and retinol;
galectin-4 and lactose;
galectin-8 and lactose; or
trbp111 and tRNA.

12. The recombinant polypeptide of claim 1, wherein:
the at least one modification of the D2 domain comprises substitution of the D2 domain with an endolysin; and
the target molecule is a bacterial peptidoglycan.

13. The recombinant polypeptide of claim 12, wherein the bacterial peptidoglycan is from Clostridium difficile, Streptococcus pyogenes, Streptococcus uberis, Streptococcus equi, Streptococcus gordinii, Streptococcus intermedius, Streptococcus parasanguis, Streptococcus pneumoniae, Enterococcus faecalis, Enterococcus faecium, Staphylococcus aureus, Bacillus anthracis, Bacillus cereus, Bacillus thuringiensis or Bacillus megaterium.

14. The recombinant polypeptide of claim 1, wherein the at least one modification of the D2 domain comprises substitution of the D2 domain with a fluorescent protein.

15. (canceled)

16. The recombinant polypeptide of claim 1, further comprising polymeric IgA or polymeric IgM.

17. The recombinant polypeptide of claim 16, wherein:
The polymeric IgA is dimeric IgA;
the polymeric IgA specifically binds a mucosal antigen; and/or
the polymeric IgA specifically binds a mucosal antigen and the mucosal antigen is a mucin.

18-19. (canceled)

20. The recombinant polypeptide of claim 1, wherein:
the amino acid sequence of the polypeptide is at least 90% identical to any one of SEQ ID NOs: 2-29, 114 and 115, or
the amino acid sequence of the polypeptide comprises any one of SEQ ID NOs: 2-29, 114 and 115.

21. (canceled)

22. A method of treating or inhibiting a Clostridium difficile, Salmonella enterica, Salmonella Tm, Streptococcus pneumoniae, Staphylococcus aureus or Campylobacter Jejuni infection in a subject, comprising administering to the subject a therapeutically or prophylactically effective amount of the recombinant polypeptide of claim 5, thereby treating or inhibiting the infection.

23. A method of treating or inhibiting an HIV-1, SARS-CoV-2, influenza virus or norovirus infection in a subject, comprising administering to the subject a therapeutically or prophylactically effective amount of the recombinant polypeptide of claim 8, thereby treating or inhibiting the infection.

24. The method of claim 22, wherein the recombinant polypeptide is administered orally, intranasally or as a suppository.

25. The method of claim 23, wherein the recombinant polypeptide is administered orally, intranasally or as a suppository.
